SEQUENCE LISTING (45 sequences)

SEQ ID NO: 1 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 2 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 3 - protein, 281 aa, Mus musculus
MFSGLTLNCVLLLLQLLLARSLEDGYKVEVGKNAYLPCSYTLPTSGTLVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTAIHIGVGVSAGLTLALIIGVLILKWYSCKKKKLSSLSLITLANLPPGGLANAGAVRIRSEENIYTIEENVYEVENSNEYYCYVNSQQPS

SEQ ID NO: 4 - DNA, 906 bp, Homo sapiens
atgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatag

SEQ ID NO: 5 - DNA, 21 bp, Artificial Sequence
  Description of Artificial Sequence Synthetic oligonucleotide
aaggtcctcagaagtggaata

SEQ ID NO: 6 - DNA, 21 bp, Artificial Sequence
  Description of Artificial Sequence Synthetic oligonucleotide
aagtggaatacagagcggagg

SEQ ID NO: 7 - DNA, 21 bp, Artificial Sequence
  Description of Artificial Sequence Synthetic oligonucleotide
aagcctcatctctttggccaa

SEQ ID NO: 8 - DNA, 21 bp, Artificial Sequence
  Description of Artificial Sequence Synthetic oligonucleotide
aaggagcctgtcctgtgtttg

SEQ ID NO: 9 - DNA, 21 bp, Artificial Sequence
  Description of Artificial Sequence Synthetic oligonucleotide
aacctgaagttggtcatcaaa

SEQ ID NO: 10 - DNA, 21 bp, Artificial Sequence
  Description of Artificial Sequence Synthetic oligonucleotide
aaccatcagaataggcatcta

SEQ ID NO: 11 - DNA, 21 bp, Artificial Sequence
  Description of Artificial Sequence Synthetic oligonucleotide
aatggtattctcatagcaaag

SEQ ID NO: 12 - DNA, 21 bp, Artificial Sequence
  Description of Artificial Sequence Synthetic oligonucleotide
aaatatccacattggccaatg

SEQ ID NO: 13 - DNA, 21 bp, Artificial Sequence
  Description of Artificial Sequence Synthetic oligonucleotide
aacatctataccattgaagag

SEQ ID NO: 14 - DNA, 21 bp, Artificial Sequence
  Description of Artificial Sequence Synthetic oligonucleotide
aatatccacattggccaatga

SEQ ID NO: 15 - DNA, 21 bp, Artificial Sequence
  Description of Artificial Sequence Synthetic oligonucleotide
ggtcctcagaagtggaataca

SEQ ID NO: 16 - DNA, 21 bp, Artificial Sequence
  Description of Artificial Sequence Synthetic oligonucleotide
ggtcggtcagaatgcctatct

SEQ ID NO: 17 - DNA, 21 bp, Artificial Sequence
  Description of Artificial Sequence Synthetic oligonucleotide
ggactgatgaaagggatgtga

SEQ ID NO: 18 - DNA, 21 bp, Artificial Sequence
  Description of Artificial Sequence Synthetic oligonucleotide
gtccctgaccatagagaatgt

SEQ ID NO: 19 - DNA, 21 bp, Artificial Sequence
  Description of Artificial Sequence Synthetic oligonucleotide
gatccaaatcccaggcataat

SEQ ID NO: 20 - DNA, 21 bp, Artificial Sequence
  Description of Artificial Sequence Synthetic oligonucleotide
gcctccctgatataaatctaa

SEQ ID NO: 21 - DNA, 21 bp, Artificial Sequence
  Description of Artificial Sequence Synthetic oligonucleotide
gccaatgacttacgggactct

SEQ ID NO: 22 - DNA, 21 bp, Artificial Sequence
  Description of Artificial Sequence Synthetic oligonucleotide
gctggctctggctcttatctt

SEQ ID NO: 23 - DNA, 21 bp, Artificial Sequence
  Description of Artificial Sequence Synthetic oligonucleotide
ggattggcaaatgcagtagca

SEQ ID NO: 24 - DNA, 21 bp, Artificial Sequence
  Description of Artificial Sequence Synthetic oligonucleotide
gcagagggaattcgctcagaa

SEQ ID NO: 25 - DNA, 21 bp, Artificial Sequence
  Description of Artificial Sequence Synthetic oligonucleotide
gagggaattcgctcagaagaa

SEQ ID NO: 26 - DNA, 21 bp, Artificial Sequence
  Description of Artificial Sequence Synthetic oligonucleotide
gtggaggagcccaatgagtat

SEQ ID NO: 27 - DNA, 21 bp, Artificial Sequence
  Description of Artificial Sequence Synthetic oligonucleotide
gagcctccctgatataaattt

SEQ ID NO: 28 - DNA, 21 bp, Artificial Sequence
  Description of Artificial Sequence Synthetic oligonucleotide
gtgctcaggactgatgaaatt

SEQ ID NO: 29 - DNA, 21 bp, Artificial Sequence
  Description of Artificial Sequence Synthetic oligonucleotide
ggtcctcagaagtggaatatt

SEQ ID NO: 30 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 31 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 32 - protein, 134 aa, Mus musculus
MDTCHIAKSCVLILLVVLLCAERAQGLECYNCIGVPPETSCNTTTCPFSDGFCVALEIEVIVDSHRSKVKSNLCLPICPTTLDNTEITGNAVNVKTYCCKEDLCNAAVPTGGSSWTMAGVLLFSLVSVLLQTFL

SEQ ID NO: 33 - protein, 886 aa, Homo sapiens
MRGFNLLLFWGCCVMHSWEGHIRPTRKPNTKGNNCRDSTLCPAYATCTNTVDSYYCACKQGFLSSNGQNHFKDPGVRCKDIDECSQSPQPCGPNSSCKNLSGRYKCSCLDGFSSPTGNDWVPGKPGNFSCTDINECLTSSVCPEHSDCVNSMGSYSCSCQVGFISRNSTCEDVDECADPRACPEHATCNNTVGNYSCFCNPGFESSSGHLSFQGLKASCEDIDECTEMCPINSTCTNTPGSYFCTCHPGFAPSNGQLNFTDQGVECRDIDECRQDPSTCGPNSICTNALGSYSCGCIAGFHPNPEGSQKDGNFSCQRVLFKCKEDVIPDNKQIQQCQEGTAVKPAYVSFCAQINNIFSVLDKVCENKTTVVSLKNTTESFVPVLKQISTWTKFTKEETSSLATVFLESVESMTLASFWKPSANITPAVRTEYLDIESKVINKECSEENVTLDLVAKGDKMKIGCSTIEESESTETTGVAFVSFVGMESVLNERFFKDHQAPLTTSEIKLKMNSRVVGGIMTGEKKDGFSDPIIYTLENIQPKQKFERPICVSWSTDVKGGRWTSFGCVILEASETYTICSCNQMANLAVIMASGELTMDFSLYIISHVGIIISLVCLVLAIATFLLCRSIRNHNTYLHLHLCVCLLLAKTLFLAGIHKTDNKMGCAIIAGFLHYLFLACFFWMLVEAVILFLMVRNLKVVNYFSSRNIKMLHICAFGYGLPMLVVVISASVQPQGYGMHNRCWLNTETGFIWSFLGPVCTVIVINSLLLTWTLWILRQRLSSVNAEVSTLKDTRLLTFKAFAQLFILGCSWVLGIFQIGPVAGVMAYLFTIINSLQGAFIFLIHCLLNGQVREEYKRWITGKTKPSSQSQTSRILLSSMPSASKTG

SEQ ID NO: 34 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 35 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 36 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 37 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 38 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 39 - protein, 556 aa, Homo sapiens [sequence omitted]

SEQ ID NO: 40 - protein, 756 aa, Homo sapiens
MYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKKKK

SEQ ID NO: 41 - protein, 448 aa, Homo sapiens
MSASGPEAPGDIPALPPPPQPGSGPAPPAPAAAAQEAMDGRAELPAFPRAGAPPLAASDTVPAAPEGAGAARPAAPLRPTSFSVLDILDPNKFNSRRRRCVLLGPVAPAACAPCASAPCAPAPAASGRPPRAEELERRALAGAGGVGAAGAEPPNAGDPFKAGEAETNDTNGYSSGGGGHSPSADSGDEVPDDEDDDEDEAPETEAARGAEEARGGGGGLGARGSGCQGAAETDASPGATVDEAAAPGPRENSPVAQGPPGGAAAPGGAGTTPQGTATAAKPKRKRTGSDSKSGKPRRARTAFTYEQLVALENKFKATRYLSVCERLNLALSLSLTETQVKIWFQNRRTKWKKQNPGADTSAPTGGGGGPGPGAGPGTGLPGGLSPLSPSPPMGAPLGMHGPAGYPAHGPGGLVCAAQLPFLSSPAVLSPFVLGSQTYGAPAFYAPHL

SEQ ID NO: 42 - protein, 296 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 43 - protein, 1163 aa, Homo sapiens
MTRTRAALLLFTALATSLGFNLDTEELTAFRVDSAGFGDSVVQYANSWVVVGAPQKITAANQTGGLYQCGYSTGACEPIGLQVPPEAVNMSLGLSLASTTSPSQLLACGPTVHHECGRNMYLTGLCFLLGPTQLTQRLPVSRQECPRQEQDIVFLIDGSGSISSRNFATMMNFVRAVISQFQRPSTQFSLMQFSNKFQTHFTFEEFRRSSNPLSLLASVHQLQGFTYTATAIQNVVHRLFHASYGARRDAAKILIVITDGKKEGDSLDYKDVIPMADAAGIIRYAIGVGLAFQNRNSWKELNDIASKPSQEHIFKVEDFDALKDIQNQLKEKIFAIEGTETTSSSSFELEMAQEGFSAVFTPDGPVLGAVGSFTWSGGAFLYPPNMSPTFINMSQENVDMRDSYLGYSTELALWKGVQSLVLGAPRYQHTGKAVIFTQVSRQWRMKAEVTGTQIGSYFGASLCSVDVDSDGSTDLVLIGAPHYYEQTRGGQVSVCPLPRGWRRWWCDAVLYGEQGHPWGRFGAALTVLGDVNGDKLTDVVIGAPGEEENRGAVYLFHGVLGPSISPSHSQRIAGSQLSSRLQYFGQALSGGQDLTQDGLVDLAVGARGQVLLLRTRPVLWVGVSMQFIPAEIPRSAFECREQVVSEQTLVQSNICLYIDKRSKNLLGSRDLQSSVTLDLALDPGRLSPRATFQETKNRSLSRVRVLGLKAHCENFNLLLPSCVEDSVTPITLRLNFTLVGKPLLAFRNLRPMLAADAQRYFTASLPFEKNCGADHICQDNLGISFSFPGLKSLLVGSNLELNAEVMVWNDGEDSYGTTITFSHPAGLSYRYVAEGQKQGQLRSLHLTCDSAPVGSQGTWSTSCRINHLIFRGGAQITFLATFDVSPKAVLGDRLLLTANVSSENNTPRTSKTTFQLELPVKYAVYTVVSSHEQFTKYLNFSESEEKESHVAMHRYQVNNLGQRDLPVSINFWVPVELNQEAVWMDVEVSHPQNPSLRCSSEKIAPPASDFLAHIQKNPVLDCSIAGCLRFRCDVPSFSVQEELDFTLKGNLSFGWVRQILQKKVSVVSVAEITFDTSVYSQLPGQEAFMRAQTTTVLEKYKVHNPTPLIVGSSIGGLLLLALITAVLYKVGFFKRQYKEMMEEANGQIAPENGTQTPSPPSEK

SEQ ID NO: 44 - protein, 1179 aa, Homo sapiens
MWLFHTLLCIASLALLAAFNVDVARPWLTPKGGAPFVLSSLLHQDPSTNQTWLLVTSPRTKRTPGPLHRCSLVQDEILCHPVEHVPIPKGRHRGVTVVRSHHGVLICIQVLVRRPHSLSSELTGTCSLLGPDLRPQAQANFFDLENLLDPDARVDTGDCYSNKEGGGEDDVNTARQRRALEKEEEEDKEEEEDEEEEEAGTEIAIILDGSGSIDPPDFQRAKDFISNMMRNFYEKCFECNFALVQYGGVIQTEFDLRDSQDVMASLARVQNITQVGSVTKTASAMQHVLDSIFTSSHGSRRKASKVMVVLTDGGIFEDPLNLTTVINSPKMQGVERFAIGVGEEFKSARTARELNLIASDPDETHAFKVTNYMALDGLLSKLRYNIISMEGTVGDALHYQLAQIGFSAQILDERQVLLGAVGAFDWSGGALLYDTRSRRGRFLNQTAAAAADAEAAQYSYLGYAVAVLHKTCSLSYVAGAPQYKHHGAVFELQKEGREASFLPVLEGEQMGSYFGSELCPVDIDMDGSTDFLLVAAPFYHVHGEEGRVYVYRLSEQDGSFSLARILSGHPGFTNARFGFAMAAMGDLSQDKLTDVAIGAPLEGFGADDGASFGSVYIYNGHWDGLSASPSQRIRASTVAPGLQYFGMSMAGGFDISGDGLADITVGTLGQAVVFRSRPVVRLKVSMAFTPSALPIGFNGVVNVRLCFEISSVTTASESGLREALLNFTLDVDVGKQRRRLQCSDVRSCLGCLREWSSGSQLCEDLLLMPTEGELCEEDCFSNASVKVSYQLQTPEGQTDHPQPILDRYTEPFAIFQLPYEKACKNKLFCVAELQLATTVSQQELVVGLTKELTLNINLTNSGEDSYMTSMALNYPRNLQLKRMQKPPSPNIQCDDPQPVASVLIMNCRIGHPVLKRSSAHVSVVWQLEENAFPNRTADITVTVTNSNERRSLANETHTLQFRHGFVAVLSKPSIMYVNTGQGLSHHKEFLFHVHGENLFGAEYQLQICVPTKLRGLQVAAVKKLTRTQASTVCTWSQERACAYSSVQHVEEWHSVSCVIASDKENVTVAAEISWDHSEELLKDVTELQILGEISFNKSLYEGLNAENHRTKITVVFLKDEKYHSLPIIIKGSVGGLLVLIVILVILFKCGFFKRKYQQLNLESIRKAQLKSENLLEEEN

SEQ ID NO: 45 - protein, 40 aa, Artificial Sequence
  Description of Artificial Sequence Synthetic polypeptide
  SITE (1)..(40): This sequence may encompass 1-8 “Gly Gly Gly Gly Ser” repeating units
GGGGSGGGGSGGGGSGGGGSGGGGSGGGGSGGGGSGGGGS

(19) United States
(12) Patent Application Publication
(10) Pub. No.: US 2024/0262909 A1
(43) Pub. Date: Aug. 8, 2024

(54) TIM-3 MODULATES ANTI-TUMOR IMMUNITY BY REGULATING INFLAMMASOME ACTIVATION

(71) Applicants: THE BRIGHAM AND WOMEN'S HOSPITAL, INC., Boston, MA (US); THE BROAD INSTITUTE, INC, Cambridge, MA (US); MASSACHUSETTS INSTITUTE OF TECHNOLOGY, Cambridge, MA (US)

(72) Inventors: Vijay K. KUCHROO, Newton, MA (US); Karen Olivia DIXON, Boston, MA (US); Marcin TABAKA, Cambridge, MA (US); AVIV REGEV, Cambridge, MA (US)

(73) Assignees: THE BRIGHAM AND WOMEN'S HOSPITAL, INC., Boston, MA (US); THE BROAD INSTITUTE, INC, Cambridge, MA (US); MASSACHUSETTS INSTITUTE OF TECHNOLOGY, Cambridge, MA (US)

(21) Appl. No.: 18/566,343

(22) PCT Filed: Jun. 3, 2022
(86) PCT No.: PCT/US2022/032093

§ 371 Date: Dec. 1, 2023

Related U.S. Application Data
(60) Provisional application No. 63/196,822, filed on Jun. 4, 2021.

(51) Int. Cl. C07K 16/28; A61K 39/00; A61K 47/69; C12N 15/113

(52) U.S. Cl. CPC C07K 16/2803; A61K 47/6931; C12N 15/113; A61K 2039/505; C12N 2310/11; C12N 2310/20

(57) ABSTRACT

Provided herein are methods and compositions for selectively promoting inflammasome activity in myeloid cells.

### CROSS-REFERENCE TO RELATED APPLICATIONS

This application is a 35 U.S.C. § 371 National Phase Entry Application of International Application No. PCT/US2022/032093 filed Jun. 3, 2022, which designates the U.S. and claims benefit under 35 U.S.C. § 119(e) to U.S. Provisional Application No. 63/196,822 filed Jun. 4, 2021, the contents of which are incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 23, 2022, is named 043214-098180WOPT_SL.txt and is 94,827 bytes in size.

### BACKGROUND

Tim-3 is a checkpoint molecule which was initially identified on IFN-γ secreting CD4+ T helper (Th1) and CD8+ Cytotoxic T cells.¹ Tim-3 has emerged as an important checkpoint molecule whose function has been well established in effector CD8+ T cells in the context of anti-tumor immunity. Expression of Tim-3 on CD8+ T cells antagonizes the maintenance of TCF1+CXCR5+ “stem-like” CD8+ T cells, such that expression of Tim-3 correlates with terminal differentiation and exhaustion in tumors^{1,2} and in chronic viral infection.^{3,4} In addition to CD8+ T cells and Th1 cells, Tim-3 has also been shown to play an important role in regulating the function of FoxP3+ regulatory T cells (Tregs), promoting CD8+ T cell dysfunction in the tumor-microenvironment (TME).⁵ A number of clinical trials are under way using blocking anti-Tim-3 monoclonal antibodies (mAbs) to re-invigorate anti-tumor immunity, and to curtail induction of T cell exhaustion.

### SUMMARY

The methods and compositions provided herein are based, in part, on the discovery that activation of the inflammasome specifically in myeloid cells can be used in the treatment of cancer. Accordingly, provided herein are methods and compositions relating to the targeted inhibition of TIM-3 in myeloid cells to increase inflammasome activity.

Provided herein, in one aspect, is a composition for selectively promoting inflammasome activity in myeloid cells, the composition comprising a TIM-3 inhibitor linked to an agent that specifically binds a myeloid cell surface marker.

In one embodiment of this aspect and all other aspects provided herein, the myeloid cell is a myeloid progenitor cell, a basophil, a neutrophil, an eosinophil, a monocyte, a macrophage, a dendritic cell, a granulocyte, a megakaryocyte or any combination thereof.

In another embodiment of this aspect and all other aspects provided herein, the TIM-3 inhibitor specifically binds to TIM-3.

In another embodiment of this aspect and all other aspects provided herein, the TIM-3 inhibitor in the composition more efficiently promotes myeloid cell inflammasome activity than the TIM-3 inhibitor not linked to the agent that binds a myeloid cell surface marker.

In another embodiment of this aspect and all other aspects provided herein, the TIM-3 inhibitor in the composition more efficiently promotes tumor cell death than the TIM-3 inhibitor not linked to the agent that binds a myeloid cell surface marker.

In another embodiment of this aspect and all other aspects provided herein, the tumor cell death comprises pyroptosis.

In another embodiment of this aspect and all other aspects provided herein, the TIM-3 inhibitor comprises an antibody or antigen-binding fragment thereof that specifically binds TIM-3.

In another embodiment of this aspect and all other aspects provided herein, the antibody or antigen-binding fragment thereof binds an epitope on the extracellular domain of TIM-3.

In another embodiment of this aspect and all other aspects provided herein, the TIM-3 inhibitor promotes degradation of TIM-3 or RNA encoding TIM-3.

In another embodiment of this aspect and all other aspects provided herein, the TIM-3 inhibitor comprises an RNA interference (RNAi) molecule, an antisense molecule, or a small molecule.

In another embodiment of this aspect and all other aspects provided herein, the TIM-3 inhibitor is in or on a nanoparticle.

In another embodiment of this aspect and all other aspects provided herein, the myeloid cell surface marker is selected from CD47, CD11b and CD11c.

Another aspect provided herein relates to a pharmaceutical composition comprising the composition as described herein and a pharmaceutically-acceptable carrier.

Also provided herein, in another aspect, is a nanoparticle comprising a TIM-3 inhibitor in or on the nanoparticle.

In one embodiment of this aspect and all other aspects provided herein, the TIM-3 inhibitor comprises a nucleic acid, a peptide or a small molecule.

In another embodiment of this aspect and all other aspects provided herein, the TIM-3 inhibitor comprises an antibody or antigen-binding fragment thereof that specifically binds TIM-3.

In another embodiment of this aspect and all other aspects provided herein, the TIM-3 inhibitor comprises a nucleic acid that promotes degradation of RNA encoding TIM-3.

In another embodiment of this aspect and all other aspects provided herein, the nucleic acid is selected from an RNAi molecule, an miRNA, a CRISPR/Cas gRNA, and an antisense molecule.

In another embodiment of this aspect and all other aspects provided herein, the nanoparticle comprises a lipid nanoparticle.

In another embodiment of this aspect and all other aspects provided herein, the nanoparticle further comprises an agent that specifically binds to a myeloid cell surface marker.

Another aspect provided herein relates to a method of promoting inflammasome activity in a myeloid cell, the method comprising contacting myeloid cell with a composition as described herein.

In one embodiment of this aspect and all other aspects provided herein, the inflammasome activity is induced to a greater extent than induced by a TIM-3 inhibitor lacking an agent that specifically binds a myeloid cell surface marker.

In another embodiment of this aspect and all other aspects provided herein, the myeloid cell is a myeloid progenitor cell, a basophil, a neutrophil, an eosinophil, a monocyte, a macrophage, a dendritic cell, a granulocyte, a megakaryocyte or any combination thereof.

In another embodiment of this aspect and all other aspects provided herein, the myeloid cell is in a solid tumor microenvironment.

Another aspect provided herein relates to a method of promoting cancer cell death, the method comprising contacting a myeloid cell associated with the cancer cell with a composition as described herein.

In another embodiment of this aspect and all other aspects provided herein, the inflammasome activity is induced to a greater extent than induced by a TIM-3 inhibitor lacking to an agent that specifically binds a myeloid cell surface marker.

In another embodiment of this aspect and all other aspects provided herein, the myeloid cell is a myeloid progenitor cell, a basophil, a neutrophil, an eosinophil, a monocyte, a macrophage, a dendritic cell, a granulocyte, a megakaryocyte or any combination thereof.

In another embodiment of this aspect and all other aspects provided herein, the cancer is acute myeloid leukemia (AML) or a solid tumor.

In another embodiment of this aspect and all other aspects provided herein, the cancer cells do not express TIM-3.

Another aspect provided herein relates to a method of treating cancer, the method comprising administering a composition as described herein to a subject in need thereof.

In one embodiment of this aspect and all other aspects provided herein, the inflammasome activity in cancer-associated myeloid cells is induced to a greater extent than induced by a non-targeted TIM-3 inhibitor.

In another embodiment of this aspect and all other aspects provided herein, the myeloid cell is a myeloid progenitor cell, a basophil, a neutrophil, an eosinophil, a monocyte, a macrophage, a dendritic cell, a granulocyte, a megakaryocyte or any combination thereof.

In another embodiment of this aspect and all other aspects provided herein, the cancer is acute myeloid leukemia (AML), chronic myeloid leukemia (CML) or a solid tumor.

In another embodiment of this aspect and all other aspects provided herein, cells of the cancer do not express TIM-3.

In another embodiment of this aspect and all other aspects provided herein, death of cells of the cancer is induced to a greater extent than induced by a TIM-3 inhibitor that is not linked to an agent that specifically binds a myeloid cell surface marker.

In another embodiment of this aspect and all other aspects provided herein, the cancer is a solid tumor.

In another embodiment of this aspect and all other aspects provided herein, the microenvironment of the solid tumor is rendered less hostile to T cells by the administering.

In another embodiment of this aspect and all other aspects provided herein, the cancer is metastatic.

In another embodiment of this aspect and all other aspects provided herein, the cancer is angiogenic.

Another aspect provided herein relates to a composition comprising a TIM-3 inhibitor linked to an agent that specifically binds a myeloid cell surface marker for use in promoting inflammasome activity or treating cancer in a subject.

In one embodiment of this aspect and all other aspects provided herein, the TIM-3 inhibitor specifically binds to TIM-3.

In another embodiment of this aspect and all other aspects provided herein, the TIM-3 inhibitor in the composition more efficiently promotes myeloid cell inflammasome activity than the TIM-3 inhibitor not linked to the agent that binds a myeloid cell surface marker.

In another embodiment of this aspect and all other aspects provided herein, the TIM-3 inhibitor in the composition more efficiently promotes tumor cell death than the TIM-3 inhibitor not linked to the agent that binds a myeloid cell surface marker.

In another embodiment of this aspect and all other aspects provided herein, the TIM-3 inhibitor comprises an antibody or antigen-binding fragment thereof that specifically binds TIM-3.

In another embodiment of this aspect and all other aspects provided herein, the antibody or antigen-binding fragment thereof binds an epitope on the extracellular domain of TIM-3.

In another embodiment of this aspect and all other aspects provided herein, the TIM-3 inhibitor promotes degradation of TIM-3 or RNA encoding TIM-3.

In another embodiment of this aspect and all other aspects provided herein, the TIM-3 inhibitor comprises an RNA interference (RNAi) molecule, an antisense molecule, or a small molecule.

In another embodiment of this aspect and all other aspects provided herein, the TIM-3 inhibitor is in or on a nanoparticle.

In another embodiment of this aspect and all other aspects provided herein, the myeloid cell surface marker is selected from CD47, CD11b and CD11c.

In another embodiment of this aspect and all other aspects provided herein, the composition further comprises a pharmaceutically acceptable carrier.

Another aspect provided herein relates to a composition comprising a TIM-3 inhibitor in or on the nanoparticle for use in promoting inflammasome activity or treating cancer in a subject.

### BRIEF DESCRIPTION OF THE DRAWINGS

FIGS. 1A-1P Deletion of Tim3 on DC leads to reduced tumor burden. MC38-OVA (0.5×10⁶ cells) were subcutaneously implanted into Tim3^{fl.fl} and (FIG. 1A) Tim3^{fl.fl}CD4 cre, (FIG. 1B) Tim3^{fl.fl}E8icre, (FIG. 1C) tamoxifen inducible Tim3^{fl.fl}Foxp3ERT2 cre and (FIG. 1D) Tim3fl.fl NCR1 cre mice and tumor growth was measured over time. (FIG. 1E) MC38-OVA (0.5×10⁶ cells) were subcutaneously implanted into WT mice and on D14 tumors were explanted followed by cell sorting for sc-RNAseq of CD45+ cells from tumor and draining LN (dLN). (top) Cell clustering and Havcr2 expression per cluster, rights violin plot of Havcr2 expression in dLN clusters, (bottom) sc-RNAseq of CD45+ cells from WT tumor with cell connotation (left) and Havcr2 expression (right); violin plot from scRNAseq displaying normalized expression of Havcr2 in each cluster, (FIGS. 1F, 1G) WT mice were implanted MC38 cells (1.0×10⁶). On D21 tumors were explanted followed by flow cytometric analysis of Tim3 (gMFI) on tumor infiltrating immune cells. (FIG. 1H) Tumor growth curve of MC38-OVA (0.5×10⁶) subcutaneously implanted Tim3^{fl.fl} and Tim3^{fl.fl}LysM cre (FIG. 11) Tim3^{fl.fl} and Tim3^{fl.fl}CX3CR1 cre and (FIG. 1J) Tim3^{fl.fl} and Tim3^{fl.fl}×CD11c cre. (FIG. 1K) Lung tumor cells were injected into WT (Tim3^{fl.fl}) and Tim3^{fl.fl}CD11c cre animals intravenously (i.v. 2.5×10⁵ cells in 100 μl PBS) to develop orthotopic tumors. Evaluation of lung tumor burden was assessed by histological analyses based on haematoxylin and eosin (H&E) staining of explanted lung tissue harvested 22 weeks post implantation. For quantification tumor area was calculated as a percentage of area occupied by the tumor as a part of total lung tissue. (FIG. 1L) WT mice were subcutaneously implanted with B16-OVA (0.25×10⁶). In vitro derived DC1 were generated from Tim3^{fl.fl} and Tim3^{fl.fl}CD11c cre in the presence of FLT3L and sorted on D10 (Sirpα-XCR1+), followed by a 2-hour culture with soluble OVA protein. Cells were harvested and 0.3×10⁶ cells were injected subcutaneously, adjacent to the palpable tumors of recipient animals. Cell transfer was conducted on D3 when palpable tumors were formed and reached ˜30 mm² across the different groups. PBS was injected into control animals for each respective group as the non-transfer control. (FIG. 1M) Tumor growth curve of MC38-OVA (0.5×10⁶) subcutaneously implanted Tim3^{fl.fl} and Tim3^{fl.fl} Zbtb46 cre, (FIG. IN) Tumor growth curve of MC38 (1.0×10⁶) subcutaneously implanted Tim3^{fl.fl} and Tim3^{fl.fl}Zbtb46 cre, (FIG. 10) Tumor growth curve of MC38-OVA subcutaneously implanted Tim3^{fl.fl}, Tim3^{fl.fl}CD4 cre and Tim3^{fl.fl}×CD11c cre in parallel. (FIG. 1P) Weights of tumors from FIG. 1O. The results shown are from one experiment, representative of at least three independent experiments. (FIGS. 1A-1D; 1H-1O n=4-5/group) *P<0.05; **P<0.01; ***P<0.001; ****P<0.0001 (Two-Way Anova). Data shown (FIG. 1O) as mean±s.e.m *P=0.0107; **P=0.0005 (Student Two-Tailed T test).

FIGS. 2A-2N Expansion of “stem-like” memory precursor CD8+ T cell in Tim3^{cko} tumors. MC38-OVA (0.5×10⁶ cells) were subcutaneously implanted into Tim3^{fl.fl} and Tim3^{fl.fl}xCD11c601 cre mice and on D14 tumors were explanted followed by cell sorting for sc-RNAseq of CD45+ cells from tumor. (FIG. 2A) Proportions of CD8+ T cells from Tim3^{fl.fl} and Tim3^{fl.fl}xCD11c cre tumor in scRNAseq data. (FIG. 2B) Transcripts of CD8+ T cells from Tim3^{fl.fl} and Tim3^{fl.fl}xCD11c cre tumors were assessed for enrichment of signatures of Memory, Memory Precursor, Early Activated and Effector T cells. (FIG. 2C) Flow Cytometric analysis (d14) of Tim3 and PD1 expression on CD8+ TILs from MC38-OVA and (FIG. 2D) B16-OVA bearing mice. (FIG. 2E) Frequency of CD8+PD1+ T cells in tumors from Tim3^{fl.fl} and Tim3^{fl.fl}xCD11c cre animals at d14 harvest. (FIG. 2F) Representative plots of IL-7R, CD5 and CXCR5 expression within CD8+PD1+ TILs with (FIGS. 2G-2I) data summarized (FIG. 2J) Representative histogram of TCF1 within CD8+PD1+ TILs, with data summarized (right), (FIG. 2K) Representative plot of CX3CR1 within CD8+PD1+ TILs; data summarized (right), (FIG. 2L) Representative histogram of T-bet, Ki67 within CD8+PD1+ TILs (FIG. 2M, 2N) data summarized. The results shown are from one experiment, representative of at least three independent experiments n=5/group. *P<0.05; **P<0.01; (Student Two-Tailed T test).

FIGS. 3A-3N Tim3 deficiency promotes DC functionality and enhances Antigen specific anti-tumor immunity. MC38-OVA (0.5×10⁶ cells) were subcutaneously implanted into Tim3^{fl.fl} and Tim3^{fl.fl}xCD11c cre mice and on D14 tumors were explanted followed by cell sorting for sc-RNAseq of CD45+ cells from tumor. (FIG. 3A) UMAP scRNAseq plot of annotated myeloid cell from Tim3^{fl.fl} and Tim3^{fl.fl}xCD11c cre tumors. (FIG. 3B) Heatmap showing expression of selected genes, top key indicates cluster origin, bottom key indicating WT or Tim3cko. (FIG. 3C) UMAP showing enrichment of mreg signature on cluster 8 (MigDC). (FIG. 3D) Empirical cumulative distribution function (ECDF) plot of enrichment score for mregDC signature in WT or Tim3^{cko} migratory DC, (p=9.4e−04), (FIG. 3E) ECDF plot of Class I presentation signature (p=1.01e−03), (FIG. 3F) Dot plot of selected genes from Class I presentation signature. (FIG. 3G) Frequency of OVA specific CD8+ T cells from WT and Tim3^{cko} tumors at d14. (FIGS. 3H-3L) MC38-OVA (0.5×10⁶ cells) were subcutaneously implanted into Tim3^{fl.fl} and Tim3^{fl.fl}xCD11c cre mice. On D14 tumors were harvested followed by flow cytometry analysis of intracellular cytokines and lytic molecules in CD8+ TILs (FIG. 3M) CFSE or CTV labelled splenocytes were pulsed with OVA257-264 or MOG37-46 (Irrelevant Antigen) and injected at 50:50 ratio into MC38-OVA bearing WT or Tim3cko mice. (FIG. 3N) Percentage cytotoxicity calculated as 100-(CTV/CTV+CFSE). The results shown are from one (FIG. 3H, 3I, 3M, 3N) or two (FIG. 3J, 3K, 3L) experiments, representative of at least three independent experiments n=4-10/group. *P<0.05; **P<0.01; ***P<0.001 (Student Two-Tailed T test).

FIGS. 4A-4W: Loss of Tim3 on DC promotes inflammasome activation. (FIG. 4A) Unidirectional analysis of the fraction of DCs expressing ligand X and the fraction of T cells expressing the cognate Receptor; Ligand (MigDC): Receptor (CD8) interaction from WT (left) and Tim3cko (right) tumors. (FIG. 4B) Bar plot of data from FIG. 4A. (FIG. 4C). ECDF plots showing enrichment of IL-18 signature and (FIG. 4D) IL-1 signature in WT and Tim3^{cko}CD8+ TILs. (FIG. 4E) ECDF plot of enrichment score for inflammasome signature in WT or Tim3^{cko} migratory DC, (p=1.5*10⁻⁰³), (FIG. 4F) Dot plot of selected genes from inflammasome signature. (FIG. 4G) ECDF plot of enrichment score for Oxidative Stress signature, (FIG. 4H) Dot plot of selected genes from Oxidative Stress signature. (FIG. 4I) MC38-OVA (0.5×10⁶ cells) were subcutaneously implanted into Tim3^{fl.fl} and Tim3^{fl.fl}xCD11c cre mice and on D14 tumors mononuclear cells were isolated and incubated with DHR123 for 30 minutes followed by flow-cytometric analysis of activated DHR123 as a measure of ROS activity in tumor infiltrating DC. (FIG. 4J) Tumor growth curve of MC38-OVA subcutaneously implanted Tim3^{fl.fl} and Tim3^{fl.fl}xCD11c cre treated with control vehicle or Caspase 1 inhibitor by oral gavage; dashed vertical lines indicate day of administration. (FIG. 4K) Weights of tumors from (j), (FIG. 4L) Tumor growth curve of MC38-OVA subcutaneously implanted Tim3^{fl.fl} and Tim3^{fl.fl}xCD11c cre treated with control or CRID3 by i.p. injection; dashed vertical lines indicate day of administration, (FIG. 4M) Weights of tumors from (l), (FIG. 4N) Tumor growth curve of MC38-OVA subcutaneously implanted Tim3^{fl.fl} and Tim3^{fl.fl}xCD11c cre treated with control or anti-IL-1 and anti-IL-18 every 3 days starting at D5, (FIG. 4O) Weights of tumors from (n), (FIGS. 4P-4T) Flow Cytometric analysis of CD8 TILs from Tim3^{fl.fl} and Tim3^{fl.fl}xCD11c cre treated with control or anti-IL-1 and anti-IL-18. The results shown are from one experiment, representative of at least two independent experiments n=4-5/group. ***P<0.001; ****P<0.0001 (Two-Way Anova). Data shown (FIGS. 4K, 4M, 4O, 4N-4R) as mean±s.e.m *P<0.05; **P<0.01; ***P<0.001 (Student Two-Tailed T test).

FIGS. 5A-5F Generation of conditional knockout mice for Tim-3 (FIG. 5A) Strategy for generating the Tim-3 targeting vector to target the Tim-3 allele. Boxes represent exons (E). The 5′ external probes for Southern Blot is indicated by thick line. Targeted events were identified by Southern blot analysis of Afl2-digested genomic ES cell DNAs with the 5′ flanking probe as shown in A. (FIG. 5B) MC38-OVA (0.5×10⁶ cells) were subcutaneously implanted into Tim-3^{fl.fl} and Tim-3^{fl.fl}CD11c cre mice. D14 dLNs were explanted followed by cell sorting for sc-RNAseq of CD45+ cells. UMAPs of select gene expression of Xcr1, Clec9a and Flt3, UMAP of Havcr2 expression among clusters found in dLN, violin plot from scRNAseq displaying normalized expression of Havcr2 in each cluster. (FIG. 5C) MC38-OVA (0.5×10⁶ cells) tumor cells were subcutaneously implanted into Tim3^{fl.fl}LysM cre, Tim3^{fl.fl}CX3CR1 cre and Tim3^{fl.fl}Zbtb46 cre animals. Flow Cytometric analysis of DC populations, macrophages and monocytes was performed on D14. DCs were gated as in FIG. 6: CD45⁺, CD3⁻CD19⁻NK1.1⁻, ClassII⁺CD11c⁺, Ly6c⁻CD64⁻ and DC1: CD103⁺CD11b⁻, DC2: CD11b⁺CD103⁻ MigDC CD11b⁺CD103⁺. Macrophages: CD45⁺, CD3⁻CD19⁻NK1.1⁻, ClassII^{lo} Ly6c^{lo}CD64⁺F480⁺CX3CR1⁺, monocytes ClassII^{lo} Ly6c^{hi}CD64^{lo}Ly6g⁻. Representative Flow Cytometric analysis of Tim-3 expression on DC1, DC2, MigDC, macrophages and monocytes. (FIG. 5D) Percentage expression and gMFI of Tim-3 (FIG. 5E, 5F) MC38-OVA (0.5×10⁶ cells) were subcutaneously implanted into Tim3^{fl.fl}, Tim3^{fl.fl}xCD4, Tim3^{fl.fl}xCD11c and Tim3^{fl.fl}xZbtb46 cre mice. On D14 tumors were explanted followed by flow cytometric analysis of Tim3 expression on CD4 TILs, CD8 TILs and tumor infiltrating DC1 from Tim3^{fl.fl}, Tim3^{fl.fl}xCD4cre, Tim3^{fl.fl}xCD11c cre and Tim3^{fl.fl}xZbtb46 cre. The results shown are from one experiment, representative of at least 3 independent experiments. The results shown (FIG. 5D) are from one experiment, n=3-4 per group. ***P<0.001; ****P<0.0001 (One-Way Anova). Data shown (FIG. 5F) as mean±s.e.m *P<0.05; ****P<0.0001 (Student Two-Tailed T test).

FIGS. 6A-6G: Adoptive Transfer of Tim3 deficient DC1 promotes anti-tumor immunity. (FIG. 6A) Tumor growth curve of MC38-OVA subcutaneously implanted Tim3^{fl.wt}xCD11c cre and Tim3^{fl.fl}xCD11c cre. (FIG. 6B) Tumor of WT mice implanted with B16-OVA. On D3 XCR1+ BMDC1 were sorted and pulsed with OVA and injected into tumor bearing mice. (FIGS. 6C-6G) Flow-cytometric analysis of OVA specific CD8+ T cells from tumors injected with WT or Tim3^{cko}DC1. The results shown are from one experiment, representative of at least three independent experiments, n=4-5/group. *P<0.05; **P<0.01; ***P<0.001; ****P<0.0001 (Two-Way Anova). Data shown (FIG. 6D-6G) as mean±s.e.m *P<0.05; **P<0.01 (Student Two-Tailed T test).

FIGS. 7A-7F: Deletion of Tim3 in cDC using Zbtb46 recapitulates findings using CD11c cre. (FIG. 7A) MC38-OVA (0.5×10⁶ cells) tumor cells were subcutaneously implanted into Tim3^{fl.fl} and Tim3^{fl.fl}Zbtb46 cre animals. Flow Cytometric analysis (d14) of Tim3 expression on tumor infiltrating DC1, DC2, MigDC and pDC from Tim3fl. and Tim3fl.fl xZbtb46 cre. (FIG. 7B) Tumor weight and total CD45+ cells of MC38 subcutaneously implanted Tim3^{fl.fl} and Tim3^{fl.fl}xZbtb46 cre. (FIG. 7C) Tumor growth curve of B16 subcutaneously implanted Tim3^{fl.fl} and Tim3^{fl.fl}xZbtb46 cre. Tumor growth curve of (FIG. 7D) B16F10 melanoma and (FIG. 7E) B16-OVA subcutaneously implanted Tim3^{fl.fl}, Tim3^{fl.fl}CD4 cre and Tim3^{fl.fl}xCD11c cre in parallel. (FIG. 7F) B16-OVA subcutaneously implanted Tim 3^{fl.fl}, Tim3^{fl.fl}CD4 cre and Tim3^{fl.fl}xZbtb46 cre in parallel The results shown are from one experiment, representative of at least three independent experiments (n=4-5/group). ****P<0.0001 (Two-Way Anova). Data shown (a-CD45+) as mean±s.e.m *P<0.05 (Student Two-Tailed T test).

FIGS. 8A-8M: Deficiency of Tim3 on DC leads to increased numbers of tumor infiltrating CD8+ T cells. MC38-OVA (0.5×10⁶ cells) tumor cells were subcutaneously implanted into Tim3^{fl.fl} and isolated at D14 (FIG. 8A) Gating strategy and phenotype of intratumoral myeloid cells, (FIGS. 8B-8L) Flow Cytometric quantification of immune cells in tumors from Tim3^{fl.fl} and Tim3^{fl.fl}xCD11c cre mice at d14 harvest. (FIG. 8M) Flow cytometric analysis of DCI and Mig DC from WT and Tim3^{cko} tumors at d14 harvest following in vitro stimulation for 4 hours in the presence of Brefeldin A and Monensin. Data shown (FIG. 8L) as mean±s.e.m *P<0.05 (Student Two-Tailed T test) n=5-9/group.

FIGS. 9A-9E: scRNAseq of WT and Tim3cko total CD45+ cells. (FIG. 9A) UMAP scRNAseq plot of annotated total cells from Tim3^{fl.fl} and Tim3^{fl.fl}xCD11c cre tumors. (FIG. 9B) UMAP scRNAseq plots showing select marker gene expression, (FIG. 9C) Heatmap from scRNAseq displaying normalized expression of select genes in each cluster. (FIG. 9D) UMAP scRNAseq plot showing distribution of WT and Tim3cko cells. (FIG. 9E) Bar graph showing frequency of WT and Tim3^{cko} cells in each cluster.

FIGS. 10A-10D: Expansion of CD8+PD1+ cells in Tim3cko tumors. MC38-OVA (0.5×10⁶ cells) were subcutaneously implanted into Tim3^{fl.fl} and Tim3^{fl.fl}xCD11c cre mice and harvested on D14 tumors (FIG. 10A) Quantification of absolute numbers of CD8+PD1+ TILs from Tim3^{fl.fl} and Tim3^{fl.fl}xCD11c cre tumors. Analysis of expression of PD1 versus (FIG. 10B) Tim3, Lag3 and TIGIT in CD8 TILs. (FIG. 10C) Flow cytometry (d14 harvest) of CD8 TILs from Tim3^{fl.fl} and T\im3^{fl.fl}xCD11c cre for expression of Tim3 and CXCR5. (FIG. 10D) Flow cytometry of CD8+PD1+ TILs from Tim3^{fl.fl} and Tim3^{fl.fl}xZbtb46 cre for expression of IL-7R, SLAMF6 CX3CR1, IFNγ, IL-2, TCF1, Ki67 and Tbet (bottom right) Representative histograms of data in (FIG. 10D). The results shown are from one experiment, representative of at least three independent experiments, n=5/group. *P<0.05; **P<0.01; (Student Two-Tailed T test).

FIGS. 11A-11E: Identification of tumor infiltrating myeloid cells in WT and Tim3^{cko} tumors. (FIG. 11A) UMAP scRNAseq plot of annotated total myeloid cells from Tim3^{fl.fl} and Tim3^{fl.fl}xCD11c cre tumors. (FIG. 11B) UMAP scRNAseq plots showing select marker gene expression, (FIG. 11C) Heatmap from scRNAseq displaying normalized expression of select genes in each cluster. (FIG. 11D) UMAP scRNAseq plot showing distribution of WT and Tim3cko cells. (FIG. 11E) Bar graph showing frequency of WT and Tim3cko cells in each cluster.

FIGS. 12A-12E: Decreased expression of mreg markers in Tim-3 deficient MigDC. MC38-OVA (0.5×10⁶ cells) tumor cells were subcutaneously implanted into Tim3^{fl.fl} and Tim3^{fl.fl}CD11c animals and (FIG. 12A) Flow Cytometric analysis of DC populations was performed on D14 to assess expression of described mreg markers including CD200, CD83, IL4R and OX49. The results shown are from one experiment, n=4 per group. (C—H) *P<0.05; **P<0.01; ***P<0.001; (One-Way Anova). (FIG. 12B) Tumor growth curves of MC38-OVA (0.5×10⁶ cells) subcutaneously implanted into Tim3^{fl.fl}, and Tim3^{fl.fl}CD11c mice treated with either Isotype control, anti-IL-12 (500 ug/mouse) or anti-IL-4 (25 ug/mouse). Treatment was initiated on D3 and antibodies were delivered i.p. every 3 days until experiment cessation. The results shown are from one experiment, n=5 per group. ***P<0.001; ****P<0.0001 (Two-Way Anova). (FIG. 12C) Splenic DC were sorted from Tim3^{fl.fl} and Tim3^{fl.fl}CD11c animals and cultured with dead HLA mismatched splenocytes osmotically loaded with 10 mg/ml Ova together with CTV labelled naïve OTI cells. Representative plots of CD44⁺CTV^{lo} T cells after 72-hour co-culture Mean±s.e.m of 3 individual mice. Alternatively, DC from Tim3^{fl.fl} and Tim3^{fl.fl}CD11c animals, were cultured with beads passively adsorbed with Ova together with CTV labelled naïve OTI cells. Representative plots of CD44⁺CTV^{lo} T cells after 72-hour co-culture. Mean±s.e.m of 3 individual mice, *P<0.0001 (Student Two-Tailed T test). (FIG. 12D) UMAP showing expression of Il18rl and Il18rap on cluster 7 (CD8+ T cells), with violin plots showing the differential expression of both receptors in of WT and Tim3^{cko}CD8⁺ T cells, (FIG. 12E) WT and Tim3^{cko} tumors were harvested and mechanically dissociated. Tissue supernatant was collected, and levels of cytokines were determined relative to mg protein per sample, n=4/group.

FIGS. 13A-13M: Enhanced inflammasome activation in Tim3cko DC. BMDC were differentiated in the presence of FLT3L for 10 days (FIG. 13A) Flow cytometric analysis assessing typical DC1 and DC2 markers. XCR1+ cells were sorted after 10 days of differentiation and seeded at a density of 0.25×10⁶. Sorted cells were either unstimulated or primed with LPS (1 ug/mL) for 3 hours followed by the addition of oxidized phospholipids (ox-PAPC) (100 ug/ml), pdA: dT (1 ug/ml), Flagellin (1 ug/ml), C. difficile (1 ug/ml), or Nigericin (10 mM). Following overnight cultures supernatants were harvested and ELISA was performed to detect (FIG. 13B) IL-1β and (FIG. 13C) TNF-a (non-inflammasome regulated control). The results shown are from one experiment (n=3 per group), representative of at least 3 individual experiments, *P<0.05 **P<0.01; ***P<0.001 (Student Two Tailed T-test). (FIG. 13D) Weights of B16-OVA (0.25×10⁶ cells) subcutaneously implanted into Tim3^{fl.fl}, and Tim3^{fl.fl}Zbtb46, and treated with either Isotype control (Hamster IgG and Rat IgG2a) or anti-IL-1β and anti-IL-18 (Hamster IgG and Rat IgG2a respectively), all at a dose of 8 mg/kg. (FIGS. 13D-13J) Flow Cytometric analysis of CD8⁺ TILs harvested from tumors in (FIG. 13D) upon termination of experiment (d15). The results shown are from one experiment, n=5 per group. (A, B) **P<0.01; ****P<0.0001 (Two-Way Anova). (C—H) *P<0.05; **P<0.01; ***P<0.001; (One-Way Anova). (FIG. 13K) Tim3^{fl.fl} (FIG. 13L) Tim3^{fl.fl}CD4 cre and (FIG. 13M) Tim3^{fl.fl}Zbtb46 cre mice were implanted with B16-OVA and monitored for development of a palpable tumor. On D6 when tumors reached ˜30-50 mm² mice were randomized and treated with either i) Isotype controls (IgG2a and IgG2b), ii) anti-Tim3, iii) anti PDL1 and iv) anti-Tim-3+PDL1. Anti-Tim-3 was administered at a dose of 200 ug/mouse and anti-PDL1 at a dose of 50 ug/ ml. All tumors were measured daily for the duration of the experiment. Antibody treatment was initiated on D6 and administered again on D9 and D12. Area under the curve (AUC) was calculated from graphs in (FIGS. 13K, 13L, and 13M) and represented in (FIG. 13K) Tim3^{fl.fl} (FIG. 13L) Tim3^{fl.fl}CD4 cre and (FIG. 13M) Tim3^{fl.fl}Zbtb46 cre. The results shown are from one experiment, n=4 per group. **P<0.01; ***P<0.001; ****P<0.0001 (Two-Way Anova). (Area under curve data) **P<0.01; ***P<0.001; ****P<0.0001 (One-Way Anova).

FIG. 14: TIM3 knockout cells produce more intracellular IL-1β upon lipopolysaccharide (LPS) stimulation.

FIG. 15: Tim3 KO BMDC1 cells secrete more IL-1B after LPS stim and addition of Nigericin. LPS was added at a final working concentration of 1 μg/mL. Medium was replaced with OPTI-MEM before adding agonist (low serum for pull-down and downstream processes). 1.5e6 cells plated per condition; 1 hour treatement with agonist (Nigericin); n=5 mice (pooled cell populations).

FIG. 16: Tim3 KO Cells Release More Cleaved IL-1β upon addition of Nigericin. Supernatant was pulled and incubated over night with 0.5 μg IL-1β Ab and 20 ul streptavidin beads. Beads were resuspended in 1× SDS buffer and supernatant was isolated, and full volume was directly run on the western blot. n=5 mice (pooled cell populations).

FIG. 17: Tim3 KO Cells Cleave more IL-1β upon addition of Nigericin. Protein lysate was directly isolated in 1× SDS buffer, and was directly loaded into the PAGE gel 20 ul (˜⅙^{th} of the total volume) was run on the western blot.

### DETAILED DESCRIPTION

TIM-3 (T cell Immunoglobulin and Mucin containing molecule 3) was first identified as a molecule expressed on IFN-γ producing T cells and is emerging as an important immune-checkpoint molecule whose therapeutic blockade is currently being investigated in multiple human malignancies. While expression of TIM-3 on CD8⁺ T cells in the tumor microenvironment is indicative of terminal T cell dysfunction, TIM-3 is also expressed on several other immune cells.

Provided herein are methods and compositions comprising targeted inhibition of TIM-3 in myeloid cells, such as dendritic cells, for enhancing the activity of the inflammasome. Also provided herein are methods and compositions for the treatment of cancer by inhibiting TIM-3 in myeloid cells, thereby promoting anti-tumor immunity.

### Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

As used herein, the term “TIM-3 inhibitor” refers to an agent that can reduce and/or inhibit TIM-3 expression or activity. In one embodiment, a TIM-3 inhibitor binds to TIM-3² polypeptide and inhibits TIM-3 activity. In another embodiment, the TIM-3 inhibitor can interfere with TIM-3 function or expression intracellularly, e.g., via interference with translation of mRNA encoding TIM-3. As a non-limiting example, such interference can involve targeted cleavage of TIM-3 transcripts. In another embodiment, the TIM-3 inhibitor can induce degradation of TIM-3. Exemplary agents include, but are not limited to an antibody, or antigen-binding fragment thereof, a small molecule, a peptide, polypeptide, nucleic acid, an RNAi interference (RNAi) molecule (including but not limited to a short interfering RNA (siRNA), a short hairpin RNA (shRNA) or a micro-RNA (miRNA)), antisense, an aptamer or a guide RNA for CRISPR-mediated inhibition. In some embodiments, inhibition can be effective at the transcriptional level, for example by reducing or inhibiting mRNA transcription and/or expression of TIM-3, for example, human TIM-3 (NCBI Gene ID No. 84868).

A “TIM-3 inhibitor linked to an agent that specifically binds a myeloid cell surface marker” or a “myeloid cell-targeted TIM-3 inhibitor” refers to an agent that reduces TIM-3 expression level or activity in myeloid cells by at least 20% when compared to TIM-3 expression level or activity in such myeloid cells in the absence of the inhibitor and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. An inhibitor of TIM-3 expression or activity in myeloid cells is targeted to and preferentially inhibits TIM-3 in myeloid cells. For example, such an inhibitor inhibits TIM-3 activity in myeloid cells at least 1x more strongly than in other cells expressing TIM-3 activity. In certain embodiments, a TIM-3 inhibitor inhibits TIM-3 activity in myeloid cells by at least 2×, 5×, 10×, 20×, 50×, 100× or more strongly than in other cells expressing TIM-3 activity. In one embodiment, such an inhibitor does not substantially inhibit TIM-3 in non-myeloid cells in vivo.

As used herein, “reduction” or “inhibition” does not encompass a complete inhibition or reduction as compared to a reference level. “Complete inhibition” refers to detect a 100% inhibition (i.e. expression or activity that is below detectable limits using a standard assay measuring TIM-3 expression or activity in myeloid cells).

As used herein, a “cell-surface marker” refers to any molecule that is expressed on the surface of a cell, such as a myeloid cell. Cell-surface expression usually requires that a molecule possesses a transmembrane domain. Many naturally occurring cell-surface markers are characterized among the “CD” or “cluster of differentiation” molecules. Cell-surface markers vary depending upon cell type, and often provide antigenic determinants to which antibodies can bind, e.g., for targeting of an agent to a desired cell or tissue.

As used herein, the term “intracellular marker” refers to any molecule that is expressed inside a given myeloid cell; such markers can be associated with one or more intracellular compartments such as the mitochondria, peroxisomes, endoplasmic reticulum, nucleus, cytoplasm, or the intracellular side of the cytoplasmic membrane, etc.

As used herein, a TIM-3 inhibitor agent that is “targeted to myeloid cells” will, following administration to an individual, be found in association with myeloid cells (e.g., dendritic cells or a sub-population of dendritic cells, among others) to a significantly greater extent than is associated with another cell population or fraction that expresses a given objective for inhibition. As used herein, an agent “targeted to myeloid cells” incudes a moiety that specifically binds a given myeloid cell-specific cell-surface marker or intracellular marker. An agent that is “targeted to a myeloid cell” will preferentially localize to a myeloid cell relative to the same agent lacking a moiety that targets it to myeloid cells. In this context, “preferentially localize” means the targeted TIM-3 inhibitor will localize to or be found in association with myeloid cells to an extent at least 5× greater than the same TIM-3 inhibitor lacking the targeting moiety when administered in the same agent concentration. In some embodiments, the localization to myeloid cells can be at least 10× greater, 20× greater, 50× greater, 100× greater or more, relative to localization to myeloid cells by the same agent lacking the targeting moiety. Such localization can be monitored using reagents that bind myeloid cell surface markers and, for example, fluorescently labeled, targeted TIM-3 inhibitors.

As used herein, “small molecule inhibitors” include, but are not limited to, small peptides or peptide-like molecules, soluble peptides, and non-peptidyl organic or inorganic compounds. A small molecule inhibitor or antagonist can have a molecular weight of about 100 to about 20,000 Daltons (Da), for example about 500 to about 15,000 Da, or about 1000 to about 10,000 Da.

As used herein, the terms “antibody reagent,” “antibodies” or “antigen-binding fragments thereof” include monoclonal, human, humanized or chimeric antibodies, single chain antibodies, Fab fragments, F(ab′) fragments, fragments produced by a Fab expression library, and/or antigen-binding fragments of any of the above. Antibodies can also refer to immunoglobulin molecules and immunologically active portions that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

As used herein, an antibody reagent (e.g., an antibody or antigen-binding domain thereof) specifically binds to a target biomarker present either on the cell-surface or in some cases intracellularly, with a K_{D} of 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less and binds to that target at least 100×, or 1000×, or 10,000× and preferably more strongly than it binds to an off-target or distinct cell-surface or intracellular marker. The person of ordinary skill in the art can determine appropriate conditions under which the polypeptide agents described herein selectively bind TIM-3 using any suitable methods, such as titration of the antibody reagent in a suitable cell binding assay.

As used herein, an “antigen-binding fragment” refers that portion of an antibody that is necessary and sufficient for binding to a given antigen. At a minimum, an antigen binding fragment of a conventional antibody will comprise six complementarity determining regions (CDRs) derived from the heavy and light chain polypeptides of an antibody arranged on a scaffold that permits them to selectively binds the antigen. A commonly used antigen-binding fragment includes the V_{H} and V_{L} domains of an antibody, which can be joined either via part of the constant domains of the heavy and light chains of an antibody, or, alternatively, by a linker, such as a peptide linker. Non-conventional antibodies, such as camelid antibodies have only 2 heavy chain sequences, denoted, for example V_{HH}. These can be used in a manner analogous to V_{H}/VL-containing antigen-binding fragments. Non-limiting examples of antibody fragments encompassed by the term antigen-binding fragment include: (i) a Fab fragment, having V_{L}, C_{L}, V_{H} and C_{H}1 domains; (ii) a Fab′ fragment, which is a Fab fragment having one or more cysteine residues at the C-terminus of the C_{H}1 domain; (iii) an Fd fragment having V_{H} and C_{H}1 domains; (iv) a Fd′ fragment having V_{H} and C_{H}1 domains and one or more cysteine residues at the C-terminus of the CH1 domain; (v) an Fv fragment having the V_{L} and V_{H} domains of a single arm of an antibody; (vi) a dAb fragment (Ward et al., Nature 341, 544-546 (1989)), also referred to as a single-domain antibody, which consists of a V_{H} domain and has only 3 CDRs; (vii) F(ab′)₂ fragments, a bivalent fragment including two Fab′ fragments linked by a disulphide bridge at the hinge region; (viii) single chain antibody molecules (e.g., single chain Fv; scFv) (Bird et al., Science 242:423-426 (1988); and Huston et al., PNAS (USA) 85:5879-5883 (1988)); (ix) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); and (x) “linear antibodies” comprising a pair of tandem Fd segments (V_{H}-C1-V_{H}-C_{H}1) which, together with complementary light chain polypeptides, form a pair of antigen binding regions (Zapata et al. Protein Eng. 8(10): 1057-1062 (1995); and U.S. Pat. No. 5,641,870).

An “isolated antibody” is intended to refer to an antibody that is substantially free of other antibodies having different antigenic specificities (e.g., an isolated antibody that specifically binds to TIM-3 is substantially free of antibodies that specifically bind antigens other than TIM-3). An isolated antibody that specifically binds to TIM-3 may, however, have cross-reactivity to other antigens, such as to TIM-3 molecules from other species. Moreover, an isolated antibody can be substantially free of other cellular material and/or chemicals.

As used herein, the term “specificity” refers to the number of different types of antigens or antigenic determinants to which an antibody or antibody fragment thereof as described herein can bind. The specificity of an antibody or antibody fragment thereof can be determined based on affinity and/or avidity. The affinity, represented by the equilibrium constant for the dissociation (K_{D}) of an antigen with an antigen-binding protein, is a measure of the binding strength between an antigenic determinant and an antigen-binding site on the antigen-binding protein, such as an antibody or antigen-binding fragment thereof: the less the value of the K_{D}, the stronger the binding strength between an antigenic determinant and the antigen-binding molecule. Alternatively, the affinity can also be expressed as the affinity constant (K_{A}), which is 1/K_{D}). Accordingly, an antibody or antigen-binding fragment thereof as described herein is said to be “specific for” a first target or antigen compared to a second target or antigen when it binds to the first antigen with an affinity (as described above, and suitably expressed, for example as a KD value) that is at least 10 times, such as at least 100 times, and preferably at least 1000 times, and up to 10000 times or more better than the affinity with which said amino acid sequence or polypeptide binds to another target or polypeptide. Antibody affinities can be determined, for example, by a surface plasmon resonance based assay (such as the BIACORE assay described in PCT Application Publication No. WO2005/012359); Forte Bio Octet™ analysis, enzyme-linked immunosorbent assay (ELISA); and competition assays (e.g., RIA's), for example.

As used herein, “avidity” is a measure of the strength of binding between an antigen-binding molecule (such as an antibody or antibody fragment thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (K_{D})) of 10⁻⁵ to 10⁻¹² moles/liter or less, such as 10⁻⁷ to 10⁻¹² moles/liter or less, or 10⁻⁸ to 10⁻¹² moles/liter (i.e., with an association constant (K_{A}) of 10⁵ to 10¹² liter/moles or more, such as 10⁷ to 10¹² liter/moles or 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction, the lower is its K_{D}. For example, a binding site on an antibody or portion thereof described herein will bind to the desired antigen with an affinity less than 500 nM, such as less than 200 nM, or less than 10 nM, such as less than 500 pM. Specific binding of an antigen-binding protein to an antigen or antigenic determinant can be determined in any suitable manner, including, for example, Scatchard analysis and/or competitive binding assays, such as radioimmunoassays (RIA), enzyme immunoassays (EIA) and sandwich competition assays, and the different variants thereof known in the art; as well as other techniques as known in the art and/or mentioned herein.

Accordingly, as used herein, “selectively binds” or “specifically binds” refers to the ability of an antibody or antigen-binding fragment thereof as described herein to bind to a target, such as TIM-3 or a given myeloid marker, with a K_{D} of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which polypeptide agents as described herein selectively bind the targets using any suitable methods, such as titration of a polypeptide agent in a suitable cell binding assay.

As used herein, the term “selectively inhibits” means that an agent, such as a bispecific antibody agent, inhibits, as that term is used herein, the association of a first ligand-receptor pair (e.g., TIM-3 and its cognate receptor) but does not substantially inhibit the association of a relevant second ligand-receptor pair.

As used herein, the term “bispecific polypeptide agent” refers to a polypeptide that comprises a first polypeptide domain which has a binding site that has binding specificity for a first target, and a second polypeptide domain which has a binding site that has specificity for a second target, i.e., the agent has specificity or is specific for two targets. The first target and second target are not the same, but are both present in an in vivo situation, such that one bispecific agent can encounter and simultaneously bind both targets. Due to the avidity effect of having two closely apposed binding domains, a bispecific agent, including a bispecific polypeptide agent, will bind to targets, including antigen epitopes that are, themselves, closely apposed, more strongly (i.e., with greater avidity) than the bispecific agent will bind either target or antigen when the targets or antigens are not in close apposition to the other. Such difference in avidity thereby provides a preference or selectivity of the bispecific agent, such as a bispecific polypeptide agent, that can be exploited for therapy.

As used herein, the term “multispecific polypeptide agent” refers to a polypeptide that comprises at least a first polypeptide domain having a binding site that has binding specificity for a first target, and a second polypeptide domain having a binding site that has binding specificity for a second target. A multispecific polypeptide agent can include further, e.g., third, fourth, etc. binding sites for additional targets. The various targets are not the same (i.e., are different targets (e.g., proteins)), but are each present in an in vivo situation, such that one bispecific agent can potentially encounter and potentially bind simultaneously to each of the targets. In one embodiment, the third, fourth or further binding site comprises a site that targets the multispecific agent to a desired location, e.g., via binding specificity for a cell- or tissue-specific marker. A non-limiting example of a multispecific polypeptide agent is a multispecific antibody construct. For the avoidance of doubt, a bispecific polypeptide agent is a type of multispecific polypeptide agent.

As used herein, the term “target” refers to a biological molecule (e.g., peptide, polypeptide, protein, lipid, carbohydrate, etc.) to which a polypeptide domain which has a binding site can selectively bind. The target can be, for example, an intracellular target (e.g., an intracellular protein target) or a cell surface target (e.g., a membrane protein, a receptor protein). Exemplary “target” biological molecules for the purposes of the methods and compositions described herein include TIM-3, and myeloid cell markers as described herein.

The term “universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, J. Mol. Biol. 196:910-917 (1987). The Kabat database is now also maintained on the world wide web. The compositions and methods described herein provide for the use of a single framework, or a set of such frameworks, which have been found to permit the derivation of virtually any binding specificity though variation in the hypervariable regions alone. The universal framework can be a V_{L} framework (V_{λ} or V_{κ}), such as a framework that comprises the framework amino acid sequences encoded by the human germline DPK1, DPK2, DPK3, DPK4, DPK5, DPK6, DPK7, DPK8, DPK9, DPKIO, DPK12, DPK13, DPK15, DPK16, DPK18, DPK19, DPK20, DPK21, DPK22, DPK23, DPK24, DPK25, DPK26 or DPK 28 immunoglobulin gene segment. If desired, the V_{L} framework can further comprise the framework amino acid sequence encoded by the human germline JK1, JK2, JK3, JK4, or JK5 immunoglobulin gene segments. In other embodiments the universal framework can be a V_{H} framework, such as a framework that comprises the framework amino acid sequences encoded by the human germline DP4, DP7, DP8, DP9, DP10, DP31, DP33, DP38, DP45, DP46, DP47, DP49, DP50, DP51, DP53, DP54, DP65, DP66, DP67, DP68 or DP69 immunoglobulin gene segments. In some embodiments, the V_{H} framework can further comprise the framework amino acid sequence encoded by the human germline J_{H}1, J_{H}2, J_{H}3, J_{H}4, J_{H}4b, J_{H}5 or J_{H}6 immunoglobulin gene segments.

An “Fv” fragment is an antibody fragment which contains a complete antigen recognition and binding site. This region consists of a dimer of one heavy and one light chain variable domain in tight association, which can be covalent in nature, for example in a single-chain Fv or scFv (see below). It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. For the methods and compositions described herein, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

A “Fab” or “Fab fragment” contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains, which permits the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10): 1057-1062 (1995). Briefly, these antibodies comprise a pair of tandem Fd segments (VH-CHI-VH-CH1) which, together with complementary light chain polypeptides, form a pair of antigen binding regions. Linear antibodies can be bispecific or monospecific.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As used herein in relation to antibody domains, “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary. Complementary domains can be found in other members of the immunoglobulin superfamily, such as the V_{α} and V_{β} (or γ and δ) domains of the T cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

The process of designing, selecting and/or preparing a bispecific of multispecific polypeptide agent as described herein is also referred to herein as “formatting” the amino acid sequence, and an amino acid sequence that is made part of a bispecific or multispecific polypeptide agent described herein is said to be “formatted” or to be in the format of that bispecific or multispecific polypeptide agent. Examples of ways in which an amino acid sequence can be formatted and examples of such formats will be clear to the skilled person based on the disclosure herein; and such formatted amino acid sequences form a further aspect of the bispecific or multispecific polypeptide agents described herein.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will ideally comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (Jones et al., Nature 321 :522-525 (1986); Riechmann et al., Nature 332:323-329 (1988); and Presta, Curr. Op. Struct. Biol 2:593-596 (1992)). The constant region, can if desired, include one or more modifications that modify or disrupt interaction of the human or humanized antibody with an Fc receptor, as described herein. Humanization can be essentially performed following the method of Winter and co-workers (Jones et al., Nature 321 :522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of autoimmune disease, cancer, or allergy. A subject can be male or female.

A subject can be one who has been previously diagnosed with or identified as suffering from or having a condition in need of treatment (e.g., cancer) or one or more complications related to such a condition, and optionally, have already undergone treatment for cancer, or the one or more complications related to cancer. Alternatively, a subject can also be one who has not been previously diagnosed as having cancer or one or more complications related thereto. For example, a subject can be one who exhibits one or more risk such diseases or disorders.

A “subject in need” of treatment for a particular condition can be a subject having that condition, diagnosed as having that condition, or at risk of developing that condition.

The term “therapeutically effective amount” refers to an amount of an inhibitor as described herein, that is effective to induce immunity against a cancer cell or tumor, thereby treating cancer. Amounts will vary depending on the specific disease or disorder, its state of progression, age, weight and gender of a subject, among other variables. Thus, it is not possible to specify an exact “effective amount”. However, for any given case, an appropriate “effective amount” can be determined by one of ordinary skill in the art using only routine experimentation.

As used herein, the terms “treat,” “treatment,” “treating,” or “amelioration” refer to therapeutic treatments, wherein the object is to reverse, alleviate, ameliorate, inhibit, slow down or stop the progression or severity of at least one symptom of cancer (e.g., tumor growth rate, tumor size, degree of angiogenesis in tumor, metastasis etc.). The term “treating” includes reducing or alleviating at least one adverse effect or symptom of a disease or disorder. Treatment is generally “effective” if one or more symptoms or clinical markers of the cancer being treated is reduced. Alternatively, treatment is “effective” if the progression of cancer is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total). The term “treatment” of a disease also includes providing relief from the symptoms or side-effects of the disease (including palliative treatment).

The terms “decrease”, “reduce”, “reduction”, or “inhibit” are all used herein to mean a decrease by a statistically significant amount. In some embodiments, “reduce,” “reduction”, “decrease” or “inhibit” means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the phrase “more efficiently promotes inflammasome activity” refers to the ability of a targeted TIM-3 inhibitor to increase inflammasome activity (as assessed, for example, using caspase-1 activity) by at least 20% compared to the inflammasome activity induced in the presence of an untargeted TIM-3 inhibitor. In other embodiments, a targeted TIM-3 inhibitor induces inflammasome activity that is at least 30%, at least 40%, at least 50%, at least 60%, at least 70%, at least 80%, at least 90%, at least 1-fold, at least 2-fold, at least 10-fold, at least 100-fold or more compared to the inflammasome activity induced by the same TIM-3 inhibitor that is not targeted (i.e., lacks the myeloid cell-specific binding agent.)

Similarly, the phrase “more efficiently promotes tumor cell death” refers to the ability of a targeted TIM-3 inhibitor to increase tumor cell death, for example, as assessed by measuring tumor size, tumor growth etc. In some embodiments, a targeted TIM-3 inhibitor induces tumor cell death to a degree that is at least 20% greater than the same TIM-3 inhibitor lacking the myeloid cell-specific targeting agent. In other embodiments, a targeted TIM-3 inhibitor induces tumor cell death to a degree that is at least 30%, at least 40%, at least 50%, at least 60%, at least 70%, at least 80%, at least 90%, at least 1-fold, at least 2-fold, at least 10-fold, at least 100-fold or more compared to the tumor cell death induced by the same TIM-3 inhibitor that is not targeted (i.e., lacks the myeloid cell-specific binding agent.)

As used herein, the terms “myeloid cell associated with a cancer cell” or “myeloid cell associated with a tumor or tumor cell” refers to a myeloid cell that is in close proximity, or in contact with a tumor or cancer cell, or a myeloid cell that is/has infiltrated into a tumor. Typically, a myeloid cell associated with a cancer or tumor cell will no longer be circulating in the blood stream and instead will be within or attached to a tumor or tumor compartment in the subject.

As used herein, the phrase “microenvironment of the solid tumor is rendered less hostile to T cells” refers to a microenvironment that is at least 20% less immunosuppressive to tumor cell- or cancer cell-associated immune cells (e.g., myeloid cells, T cells etc) in the presence of a targeted TIM-3 inhibitor as compared to the tumor microenvironment in either (i) the presence of an untargeted TIM-3 inhibitor, or (ii) in the absence of a TIM-3 inhibitor. In other embodiments, a targeted TIM-3 inhibitor reduces the immunosuppressive effect of a tumor microenvironment to a degree that permits at least 20%, at least 30%, at least 40%, at least 50%, at least 60%, at least 70%, at least 80%, at least 90%, at least 1-fold, at least 2-fold, at least 10-fold, at least 100-fold or more increase in immune cell anti-tumor activity in that microenvironment. Such activity can be measured, e.g., by monitoring levels of activated or antigen responsive immune cells in the tumor or tumor microenvironment.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The term “effective amount” as used herein is defined as an amount effective, at dosages and for periods of time necessary to achieve the desired result. The effective amount of a compound of the invention may vary according to factors such as the disease state, age, sex, and weight of the animal. Dosage regimens may be adjusted to provide the optimum therapeutic response. For example, several divided doses may be administered daily or the dose may be proportionally reduced as indicated by the exigencies of the therapeutic situation.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

### TIM-3

TIM3(T cell Immunoglobulin and Mucin domain containing molecule) is a receptor molecule selectively expressed on a subset of murine IFNγ-secreting Th1 cells, but not on Th2 cells, and regulates Th1 immunity and tolerance in vivo. In addition, TIM3 is shown in the working Examples to be expressed by myeloid cells, such as dendritic cells, among others. Inhibition of TIM3 specifically on myeloid cells can produce or permit an immune response against the tumor.

Tim-3 is classed as a type I membrane protein comprising 281 amino acids whose extracellular domain comprises an IgV-like domain followed by a mucin-like region. In humans, TIM3 is expressed by a subset of activated CD4+ cells, and antiCD3/28 stimulation increases both the level of expression as well as the number of TIM3+ T-cells. TIM3 is expressed at high levels on in vitro polarized Th1 cells, IFNγ-secreting Th1 cells, and is also constitutively expressed on dendritic cells, on peripheral macrophages, and is expressed at lower levels on Th17 cells. In addition, human CD4+ T-cells secreted elevated levels of IFN-γ, IL-17, IL-2, and IL-6, but not IL-10, IL-4, or TNFα, when stimulated with anti-CD3/28 in the presence of TIM3-specific, antagonistic antibodies, which is mediated by induction of cytokines at the transcriptional level. TIM3 is a negative regulator of human T-cells and regulates Th1 and Th 17 cytokine secretion; blockade of TIM3 with either monoclonal antibody or RNA interference agents increases the secretion of IFNγ by activated human T-cells (Hastings et al., 39 Eur. J. Immunol 2492 (2009)). TIM3 has also been shown to bind phosphatidylserine, a major “eat me” signal. TIM3 has been shown previously to utilize galectin-9 as a heterophilic ligand. Galectin-9 was identified as a TIM3 ligand that specifically recognizes carbohydrate motif(s) on the TIM3 IgV domain (Zhu et al., Nat. Immunol (2005); U.S. Patent Pub. No. 2005/0191721).

Structurally, TIM3 comprises an N-terminal IgV domain followed by a mucin domain, a transmembrane domain, and a cytoplasmic tail. The TIM3 IgV domain (human and murine) has four non-canonical cysteines that form two unique disulfide bonds, which place the CC′ and FG loops in close proximity. The surface formed by these loops form a binding cleft (FG-CC′ cleft) that is not present in other immunoglobulin superfamily (IgSF) members, and mutagenesis studies demonstrated that this surface contributes to the recognition of a non-galectin-9-ligand(s) that is present on a wide range of primary immune cells (Cao et al., 26 Immunity 311 (2007); Anderson et al., 26 Immunity 273 (2007)). Within the FG-CC′ cleft, Gln62 and Arg112 are critical for galactin-9-independent ligand binding. Substitution of Gln62 did not alter phagocytic activity, whereas substitution of Arg112 completely abrogated the activity. The metal-ion-dependent ligand binding site of TIM3, also important for recognition of apoptotic cells, requires N120 and D121, and to a lesser extent L118 and M119, present in the FG loop of the IgV domain. (Nakayama et al., 113 Blood 3821 (2009)). Thus, although a second heterophilic ligand had been predicted to be involved in TIM3 binding (i.e., to the FG-CC′ cleft), the identity and nature of this interaction remained a mystery until the discoveries described herein.

The amino acid sequences of exemplary TIM3 polypeptides have been deposited at the GenBank database under accession numbers AAL65156-AAL65158, as follows:

| GenBank Accession No. AAL65157, human TIM3, clone 1 | |
|---|---|
| (SEQ ID NO: 1) | |
| MFSHLPFDCV LLLLLLLLTR SSEVEYRAEV GQNAYLPCFY TPAAPGNLVP VCWGKGACPV | 60 |
| FECGNVVLRT DERDVNYWTS RYWLNGDFRK GDVSLTIENV TLADSGIYCC RIQIPGIMND | 120 |
| EKFNLKLVIK PAKVTPAPTL QRDFTAAFPR MLTTRGHGPA ETQTLGSLPD INLTQISTLA | 180 |
| NELRDSRLAN DLRDSGATIR IGIYIGAGIC AGLALALIFG ALIFKWYSHS KEKIQNLSLI | 240 |
| SLANLPPSGL ANAVAEGIRS EENIYTIEEN VYEVEEPNEY YCYVSSRQQP SQPLGCREAM | 300 |
| P | 301 |
| GenBank Accession No. AAL65158, human TIM3, clone 2 | |
| (SEQ ID NO: 2) | |
| MESHLPFDCV LLLLLLLLTR SSEVEYRAEV GQNAYLPCFY TPAAPGNLVP VCWGKGACPV | 60 |
| FECGNVVLRT DERDVNYWTS RYWLNGDFRK GDVSLTIENV TLADSGIYCC RIQIPGIMND | 120 |
| EKFNLKLVIK PAKVTPAPTR QRDFTAAFPR MLTTRGHGPA ETQTLGSLPD INLTQISTLA | 180 |
| NELRDSRLAN DLRDSGATIR IGIYIGAGIC AGLALALIFG ALIFKWYSHS KEKIQNLSLI | 240 |
| SLANLPPSGL ANAVAEGIRS EENIYTIEEN VYEVEEPNEY YCYVSSROOP SQPLGCRFAM | 300 |
| P | 301 |
| GenBank Accession No. AAL65156, murine TIM3 | |
| (SEQ ID NO: 3) | |
| MFSGLTLNCV LLLLQLLLAR SLEDGYKVEV GKNAYLPCSY TLPTSGTLVP MCWGKGFCPW | 60 |
| SQCTNELLRT DERNVTYQKS SRYQLKGDLN KGDVSLIIKN VTLDDHGTYC CRIQFPGLMN | 120 |
| DKKLELKLDI KAAKVTPAQT AHGDSTTASP RTLTTERNGS ETQTLVTLHN NNGTKISTWA | 180 |
| DEIKDSGETI RTAIHIGVGV SAGLTLALII GVLILKWYSC KKKKLSSLSL ITLANLPPGG | 240 |
| LANAGAVRIR SEENIYTIEE NVYEVENSNE YYCYVNSQQP S | 281 |

Such TIM-3 peptides or portions thereof can be used to generate an antibody or an antigen-binding fragment thereof for use as an inhibitor of TIM-3.

An exemplary TIM3 mRNA sequence is provided herein as follows:

| GenBank database under Accession No. JX049979.1, Homo sapiens T-cell immunoglobulin | |
|---|---|
| and mucin domain-containing protein 3 mRNA, complete cds | |
| (SEQ ID NO: 4) | |
| 1 | atgttttcac atcttccctt tgactgtgtc ctgctgctgc tgctgctact acttacaagg |
| 61 | tcctcagaag tggaatacag agcggaggtc ggtcagaatg cctatctgcc ctgcttctac |
| 121 | accccagccg ccccagggaa cctcgtgccc gtctgctggg gcaaaggagc ctgtcctgtg |
| 181 | tttgaatgtg gcaacgtggt gctcaggact gatgaaaggg atgtgaatta ttggacatcc |
| 241 | agatactggc taaatgggga tttccgcaaa ggagatgtgt ccctgaccat agagaatgtg |
| 301 | actctagcag acagtgggat ctactgctgc cggatccaaa toccaggcat aatgaatgat |
| 361 | gaaaaattta acctgaagtt ggtcatcaaa ccagccaagg tcacccctgc accgactctg |
| 421 | cagagagact tcactgcagc ctttccaagg atgcttacca ccaggggaca tggcccagca |
| 481 | gagacacaga cactggggag cctccctgat ataaatctaa cacaaatatc cacattggcc |
| 541 | aatgagttac gggactctag attggccaat gacttacggg actctggagc aaccatcaga |
| 601 | ataggcatct acatcggagc agggatctgt gctgggctgg ctctggctct tatcttcggc |
| 661 | gctttaattt tcaaatggta ttctcatagc aaagagaaga tacagaattt aagcctcatc |
| 721 | tctttggcca acctccctcc ctcaggattg gcaaatgcag tagcagaggg aattcgctca |
| 781 | gaagaaaaca tctataccat tgaagagaac gtatatgaag tggaggagcc caatgagtat |
| 841 | tattgctatg tcagcagcag gcagcaaccc tcacaacctt tgggttgtcg ctttgcaatg |
| 901 | ccatag |

TIM-3 expression level can be measured by any means known in the art, including, but not limited to RT-PCR or Western blotting, e.g., of sort-purified myeloid cells treated with an inhibitor compared to myeloid cells in the absence of the inhibitor.

TIM-3 activity level can be measured by any means known in the art, including, but not limited to, e.g., measuring in vitro activity of cytotoxic T cells (e.g., CD4+ T cells) in the presence of an inhibitor, as compared to the activity of cytotoxic T cells in the absence of the inhibitor using, for example an ELISA to detect cytokine production. Exemplary assays to determine the activity of TIM3 are disclosed in U.S. Patent Pub. No. 2005/0191721. In an example of an ex vivo assay, human monocytes are isolated by negative selection from the peripheral blood of healthy subjects using magnetic beads (Miltenyi Biotech). Monocytes (2×10⁵/well) are stimulated and cytokine production in the presence of a targeted TIM-3 inhibitor is measured after 48 hours by ELISA and compared to cytokine production in stimulated monocytes in the presence of either untargeted TIM-3 or a vehicle control.

As used herein, an “immune response” being modulated refers to a response by a cell of the immune system, such as a myeloid cell, B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

### Inflammasomes and Pyroptosis

As used herein, the term “inflammasome” refers to a multiprotein intracellular complex that detects pathogenic microorganisms and sterile stressors, and that activates the highly pro-inflammatory cytokines interleukin-1b (IL-1b) and IL-18. Inflammasomes also induce a form of cell death termed pyroptosis. Dysregulation of inflammasomes is associated with a number of autoinflammatory syndromes and autoimmune diseases.

The inflammasome activates a pyroptotic inflammatory cascade by binding to pro-caspase-1 (the precursor molecule of caspase-1), either homotypically via its own caspase activation and recruitment domain (CARD) or via the CARD of the adaptor protein ASC which it binds to during inflammasome formation. In its full form, the inflammasome promotes association of multiple p45 pro-caspase-1 molecules, inducing their autocatalytic cleavage into p20 and p10 subunits. Caspase-1 then assembles into its active form consisting of two heterodimers with a p20 and p10 subunit each. Once active, it can then carry out a variety of processes in response to the initial inflammatory signal. These include the proteolytic cleavage and activation of IL-1β, IL-18, as well as cleavage of Gasdermin-D to release its N-terminal fragment responsible for the induction of pyroptosis. IL-1β and IL-18 released following inflammasome activation can induce IFN-γ secretion and natural killer cell activation, cleavage and inactivation of IL-33, DNA fragmentation and cell pore formation, inhibition of glycolytic enzymes, activation of lipid biosynthesis and secretion of tissue-repair mediators such as pro-IL-1α.

In addition, some studies have also described non-canonical inflammasome complexes that act independently of caspase-1. In mice, the non-canonical inflammasome is activated by direct sensing of cytosolic bacterial lipopolysaccharide (LPS) by caspase-11, which subsequently induces pyroptotic cell death. In human cells, the corresponding caspases of the non-canonical inflammasome are caspase 4 and caspase 5. Recent studies have shown that activation of TLRs by select DAMPs can trigger pyroptosis, a novel caspase-1-dependent pro-inflammatory cell death (Masters S. L, et al. Immunity. 2012 37(6): 1009-23; Brennan M. A., et al. Mol Microbiol. 2000 38(1):31-40; Cookson B. T. & Brennan M. A. Trends Microbiol. 2001 9(3): 113-4) that involves the activation of ion gradients, cell swelling and the release of IL-1β and IL-18, intracellular DAMPs and other pro-inflammatory cytokines (Ehrchen J. M, et al. J Leukoc Biol. 2009 86(3):557-66; Maratheftis C. I., et al. Clin Cancer Res. 2007 13(4): 1154-60; Rhyasen G. W, et al. Cancer Cell. 2013 24(1):90-104; Masters S. L, et al. Immunity. 2012 37(6): 1009-23; Bergsbaken T, et al. Nat Rev Microbiol. 2009 7(2):99-109). Inflammasome activity can be assessed by measuring the formation of ASC (apoptosis-associated speck-like protein containing a caspase recruitment domain) or NLRP3 specks, cleavage of caspase-1, induction of IL1β protein level, or release of IL1β (e.g., by ELISA). Inflammasome activity can also be assessed by measuring expression of the main inflammasome components (NLRP3 (or other sensory molecules), ASC, and caspase-1); detection of ASC specks (unique localized 3D structures that form upon inflammasome activation); presence of cleaved/active caspase-1 enzyme; caspase-1 activity on substrates such as pro-IL-1β, pro-IL-18 and pro-gasdermin-D; cell lysis and the following increase in the level of mature IL-1β, IL-18 and gasdermin-D. Inflammasome activity can also be assessed as described in e.g., Grinstein et al. Pediatric Rheumatology 13(Suppl 1):051 (2015); Tran et al. PLOS One 14(4): e0214999; Yaron et al. Cell Death Dis 6(10): e1952 (2015); Yaron et al. Biochem Biophys Res Comm 472(3):545-550 (2016); Fernandez et al. J Vis Exp 87:51284 (2014)). Inflammasome activity assays are also available commercially from e.g., Promega (e.g., Caspase-GLO™ 1 Inflammasome Activity Assay).

In one embodiment, inflammasome activity (as assessed using e.g., a caspase 1 activity assay or IL-1β expression in a whole blood sample) is increased in the presence of a myeloid-targeted TIM-3 inhibitor by at least 20% as compared to the inflammasome activity in the absence of the inhibitor (or alternatively in the presence of a non-targeted TIM-3 inhibitor). In other embodiments, the inflammasome activity is increased by the myeloid-targeted TIM-3 inhibitor by at least 30%, at least 40%, at least 50%, at least 60%, at least 70%, at least 80%, at least 90%, at least 95%, at least 98%, at least 99%, at least 1-fold, at least 2-fold, at least 10-fold, at least 100-fold or more compared to a non-targeted TIM-3 inhibitor or an untreated control.

### Myeloid Cells and Markers Thereof

Essentially any cell in the myeloid lineage of cells can be targeted for inhibition of TIM-3 for the purpose of inducing inflammasome activity. A myeloid cell can be a myeloid stem cell, a myeloid progenitor cell, a myeloblast, a promyelocyte, a myelocyte, a metamyelocyte, an immature monocyte, an immature basophil, an immature eosinophil, myeloid progenitor cell, a basophil, a neutrophil, an eosinophil, a monocyte, a macrophage, a dendritic cell, a granulocyte, a megakaryocyte or any combination thereof. In one embodiment, the myeloid cell is a dendritic cell.

Cells of the myeloid lineage have also been shown to play a role in the development or maintenance of a tumor through modulation of the tumor microenvironment or immune response in the tumor. As used herein, an immune response being modulated refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some embodiments of the compositions and methods described herein, an immune response being modulated is T-cell tolerance.

Myeloid populations of the tumor microenvironment predominantly include monocytes and neutrophils (sometimes loosely grouped as myeloid-derived suppressor cells), macrophages, and dendritic cells. Recently, myeloid cells that are both ‘stimulatory’ and ‘non-stimulatory’ have been identified in the tumor microenvironment. In the setting of cancer, a significant excess of macrophages and dysfunctional or skewed populations of macrophages and other cell types are commonly described in the tumor microenvironment. Thus, there appears to be diversity in the antigen-presenting compartment within tumors, which modulate T cell activity to a different degree.

Myeloid cells are prominent antigen-presenting cells (APC) within the tumor itself and likely influence the functions of tumor cytotoxic T-lymphocytes (CTLs). T-cell activation by antigen presenting cells is an important component in antigen-specific immune responses and tumor cell killing. In addition, myeloid cells play an important role in tumor progression. They suppress host immune surveillance and influence the tumor microenvironment (see, e.g., Gabrilovich et al., Nat. Rev. Immunol., 9: 162-174 (2009); Pollard, Nat. Rev. Immunol., 9: 259-270 (2009); Mantovani, Nature, 457: 36-37 (2009), Fridlender et al., Cancer Cell, 16: 183-194 (2009); Balkwill et al., Nature, 431: 405-406 (2004), Yang et al., Cancer Cell, 6: 409-421 (2004); and Yang et al., Cancer Cell, 13: 23-35 (2008)). Myeloid cells in the tumor microenvironment include tumor-associated macrophages (TAM, Mac-1+ or F4/80+ cells), Gr-1+CD11b+ cells or myeloid derived suppressor cells (MDSCs), and tumor associated neutrophils (TAN, CD11b+Ly6G+ cells). One of the most important properties of these cells is the increased TGFβ production and increased Th2 polarization (see, e.g., Yang, et al., Cancer Cell, 13: 23-35 (2008); and Flavell et al., Nat. Rev. Immunol. 10: 554-567 (2010)).

In some embodiments, myeloid cells to be targeted for TIM-3 inhibition can be ‘non-stimulatory,’ such as tumor-associated macrophages; tumor-associated dendritic cells; CD45+, HLA-DR+, CD11c+, CD14+, and BDCA3−; CD45+, HLA-DR+, and CD14+; CD45+, HLA-DR+, CD14+, BDCA3−, CD11b+, and CD11c+; CD45+, HLA-DR+, CD14−, CD11c+, and BDCA1+; or are not BDCA3+, for example as may be determined by flow cytometry or an equivalent assay. In some aspects, the non-stimulatory myeloid cells are positive for at least one of: C5AR1, LYVE1, ABCC3, MRC1, SIGLEC1, STAB1, C1QB, C1QA, TMEM37, MERTK, C1QC, TMEM119, MS4A7, APOE, CYP4F18, TREM2, TLR7, and LILRB4; and/or are negative for at least one of: KIT, CCR7, BATF3, FLT3, ZBTB46, IRF8, BTLA, MYCL1, CLEC9A, BDCA3, and XCR1, for example as may be as measured by polymerase chain reaction (PCR), gene array, flow cytometry, RNAseq, or an equivalent assay.

In other embodiments, the myeloid cells to be targeted for TIM-3 inhibition using the methods and compositions described herein are ‘stimulatory myeloid cells,’ which comprise cells that are at least one of CD45+, HLA-DR+, CD14−, CD11c+, BDCA1−, and BDCA3+; CD45+, HLA-DR+, CD14−, CD11c+, and BDCA3+; CD45+, HLA-DR+, and BDCA3+; CD45+, HLA-DR+, CD14−, and BDCA3+; and CD45+, HLA-DR+, CD11c+, and BDCA3+, for example as may be determined by flow cytometry or an equivalent assay. In some aspects, the stimulatory myeloid cells are negative for at least one of: C5AR1, LYVE1, ABCC3, MRC1, SIGLEC1, STAB1, C1QB, C1QA, TMEM37, MERTK, C1QC, TMEM119, MS4A7, APOE, CYP4F18, TREM2, TLR7, and LILRB4; and/or are positive for at least one of: KIT, CCR7, BATF3, FLT3, ZBTB46, IRF8, BTLA, MYCL1, CLEC9A, BDCA3, and XCR1, for example as may be measured by polymerase chain reaction (PCR), gene array, flow cytometry, RNAseq, or an equivalent assay.

In further embodiments, a heterogeneous population of myeloid cells (i.e., stimulatory and non-stimulatory) can be targeted simultaneously for inhibition of TIM-3.

Exemplary cell-surface markers of ‘non-stimulatory myeloid cells’ include, but are not limited to, TREM2, MS4A7, C5AR1, LYVE1, ABCC3, LILRB4, MRCI/CD206, SIGLEC1, STAB1, TMEM37, MERTK, and TMEM119, wherein the non-stimulatory myeloid cells are CD45+, HLA-DR+, CD11c+, CD14+, and BDCA3−. Exemplary cell-surface markers of ‘stimulatory myeloid cells’ include, but are not limited to, C5AR1, LYVE1, ABCC3, MRC1, SIGLEC1, STAB1, C1QB, C1QA, TMEM37, MERTK, C1QC, TMEM119, MS4A7, APOE, CYP4F, 18, TREM2, TLR7, LILRB4, KIT, CCR7, BATF3, FLT3, ZBTB46, IRF8, BTLA, MYCL1, CLEC9A, BDCA3, and XCR1.

In certain embodiments, the TIM-3 inhibitor is targeted to a neutrophil by an antibody or antigen-binding fragment directed to one or more markers of neutrophils, including CD45, CD11b, and/or Ly6G. Additional exemplary neutrophil markers can include, for example, cell surface marker such as CD10, CD15, CD17, CD24, CD35, CD43, CD66a, CD66b, CD66c, CD89, CD93, CD112, G-CSFR, CD116, CD157, CD177, CXCR1, TLR2, TLR6, Ly-6G, calprotectin, and/or intracellular markers CD281, and CD289.

In certain embodiments, the TIM-3 inhibitor is targeted to monocytes by an antibody or antigen-binding fragment that binds CD45, CD11b, and/or Ly6C. Additional non-limiting examples of monocyte markers are described in US2016/0024578, the contents of which are incorporated by reference in its entirety. Such exemplary monocyte markers can include PARK2, MTMR11, TCF7L2, C18orf1, ERICH1, EHD4, CENPA, MYOF, PPM1F, FAR1, SCN1A, TRRAP, MGRN1, RBM47, KIAA0146, KAZN, RIN2, ERCC1, ANKRD11, SECTM1, DUSP1, CYB561, KCNQ1, FANCA35, FAM26F, PRKACA, TSPAN16, NAAA, ELF5, GPR152, TCF7L2, UHRF1BP1L, DDAH2, SMG6, LOC285740, RGS12, TMEM181, WIPI12, BCL6, RASA3, WDR46, LOC338779 and/or SNRPC.

In certain embodiments, the TIM-3 inhibitor is targeted to CD11b macrophages by an antibody or antigen-binding fragment that binds CD45, CD11b, and/or F4/80. Additional macrophage markers can include, but are not limited to, Ly-6c, RELM-a, CD68, IDO, IL-10, TGF-β, CD115, CD204, CD163, CD206, CD209, FceR1, VSIG4, IRF4, STAT6, arginase, YM1, CD14, and/or CSFIR.

In certain embodiments, the TIM-3 inhibitor is targeted to T cells by an antibody or antigen-binding fragment that binds CD45, CD3, CD4 and/or CD8. Additional non-limiting examples of T cell markers are described in US2016/0024578, the contents of which are incorporated by reference in its entirety. Exemplary CD8+ T cell markers can include PHRF1, SBF1, PDGFA, PCID2, KIF3C, C6orf10, SOX5, TDRD9, MYBPH, SEMA3A, DEFB114, EHD1, C14orf166, MSC, SHANK2, NINL, SGMS1, HMCN1, CTR9, NCRNA, ANKRD55, AFF3, LRRK1, PLEKHA7, AHNAK, GALR1, FSTL4, ANK3, SYNPO, MUC21, LRP5, APP, SERPIN12, LPCAT1, MED13L, PPAP2B, OR8S1, CACHD1, COL4A2, and/or EPS8. Exemplary CD4+ T cell markers can include CA6, MAN1C1, STIM2, ARHGEF2, DUSP5, ITGAX, GGA1, RAP1GDS1, GPR63, SDCCAG3, SS18L1, TALDO1, FAM38A, PON2, ALLC, HLA-DRB1, FARS2, HCFC1, NUBP1, HLA-DBR6, OSBPL5, ERICH1, PLAT, FLG2, GNRHR, PAGE2M, TRRAP, SMYD3, SMURF1, AHRR, TBCD93, RANBP3L, MCC, CD28, and/or ILA-DQB1.

In certain embodiments, the TIM-3 inhibitor is targeted to B cells by an antibody or antigen-binding fragment that binds B cells CD45, B220 and/or CD19. Additional non-limiting examples of B cell markers are described in US2016/0024578, the contents of which are incorporated by reference in its entirety. Such exemplary B cell markers can include CD19, CD20, CD40, CD45R, IgM, CYBASC3, NFATC1, TTLL10, LRP5, LOC100129637, UBE20, TRPV1, TBCD, SORL1, C7orf50, C15orf57, TERF1, BAHCC1, LRIG1, MICAL3, CDK19, GOLSYN, INPP5J, EIF3G, ITPKB, IQSEC1, IFR2, ZDHHC14, WDFY4, LCN8, PLXND1, CARS2, RERE, HVCN1, FRMD8, CGNL1, IQSEC1, RNF44, ATP10A, LHPP, CD84 and/or CD81.

In certain embodiments, the TIM-3 inhibitor is targeted to Natural killer cells (NK) cells by an antibody or antigen-binding fragment that binds CD45 and/or NK1.1. Additional exemplary NK cell markers include, but are not limited to, CD34, CD7, CD133, CD38, CD45RA, CD244, CD10, CD117, CD122, NKG2D, CD335, NKG2A, CD337, CD161, NKP80, CD127, CD56, KIR, and/or CD57. Other non-limiting examples of NK cell markers include ANKRD28, DNM3, CTBP2, RHOBTB1, LDB2, LARP4B, CXXC5, RNF165, EIF3G, EIF2C2, MYO1E, FAM120B, EIF3B, ADAM8, ZDHHC14, SLC15A4, RASA3, C1GALT1, COLQ, MAST3, MAD1L1, RFC2, AKAP10, SBN02, PDGFA, C14orf166, TBC1D22B, LDHAL6A, ST7, ZAK, NCRNA00119, TBC1D23, SAMD4A, GCK, PTK2, AOAH, C3orf30, SGMS1, RCAN2, ELFN1, UBE2E2, CLIP1, KCNQI, GPR89A, OSBPL10, IL9, TNKS2, KCNQ1, PLEKHA7 or one or more markers as described in US2016/0024578, the contents of which are incorporated herein by reference in its entirety.

In certain embodiments, the TIM-3 inhibitor is targeted to dendritic cells using markers such as, CD11c, HLA-DR, CD141, CLEC9A, CADM1, CD1c, CD11b, FCER1A, CLEC10A, CD2, CD172A, ILT-1, MHCII, ESAM, CD303, CD123, CD1a, CD206, and/or CD209.

In certain embodiments, the TIM-3 inhibitor is targeted to dendritic cells subset 1 (DC1) by an antibody or antigen-binding fragment that binds CD45, ClassII, CD11c, and/or CD103/XCR1.

In certain embodiments, the TIM-3 inhibitor is targeted to dendritic cells subset 2 (DC2) by an antibody or antigen-binding fragment that binds CD45, ClassII, CD11c, and/or CD11b.

In certain embodiments, the TIM-3 inhibitor is targeted to migratory DCI dendritic cells by an antibody or antigen-binding fragment that binds CD45, ClassII, CD11c, CD11b and/or CD103/XCR1.

In certain embodiments, the TIM-3 inhibitor is targeted to granulocytes or a subset thereof. Pan-granulocyte markers include, but are not limited to, CD11b, CD13, CD15, CD16/32, CD32 and CD33. Non-limiting examples of granulocyte markers are described in US2016/0024578, the contents of which are incorporated by reference in its entirety. Additional exemplary granulocyte cell markers can include TIMP2, DCP1A, PCMTD1, PXT1, LOC339524, UNKL, PVT1, SLPC23A2, PDE4D, NUDT3, MTIF2, GTPDP1, ANAPC10, RRM2, HNRNPUL1, NCK2, TTN, UBE2H, ZNF148, REC8, MAP7, NCAPD2, GLB1, VPS53, GRK4, TRPS1, CHD7, COL18A1, RNF103, RCOR, VKORC1L1, PBX1, ZNF609, C6orf70, CDK5RAP1, PILRB, AMPD3, MATN2, HDC, MCC, ERI3, TTLL8, ZFPM1, CLDN20, MS4A2, DENND3, DLC1, MAS1L, MAD1L1, PFKFB4, DPYSL2, FBXL14, SFSWAP, ADK, NFAT5, MEGF9, SIK2, WDFY2, TES, Clorf198, SDPR, MAP2K4, TANC1, ANXA13, ZNF366, SHB, ABCC1, LPP, LIN7A, TECR, ROR1, ARIDSB, RGL1, CPB2, RPS6KA2, TIMP2, ANXA11, ATL2, C10orf18, BBX, FARSA, CALU, C6orf89, PPP1R1B, HEXA, ETS1, MEF2A, IGF1R, C12orf43, HEXA, IL1RL1, TMEM220, PCYT1A, MARCH3, LOC100271715, C7orf36, USP20, OSTa1-pha, PCYT1A, RREB1, DKFZp761e198, EIF4EBP1, TMED3, HTT, MAN2A2, DCAF5, CHD7, MAT2B, PRKCH, TMEM156, APLP2, STX3, TSNAX-DISC1, DMXL1, NADSYN1, ENO1, MCF2L2, HIPK3, MARCH8, FAM125B, RPTOR, VPS53, CHST15, MED21, CPM, ITGAE, ANKFY1, ARG1, PCYOX1, KLF11, SH3PXD2B, DIP2C, CAST, CSGAL/NACT1, INPP5A, FOXN3, RGL1, MLTT1, DCAF4L1, HDLBP, C12orf71, NKTR, PEX5, and/or NQO2.

In certain embodiments, the TIM-3 inhibitor is targeted to basophils. Non-limiting examples of basophil markers include CD9, CD11a, CD13, CD14, CD16, CD24, CD25, CD33, CD38, CD43, CD56, CD63, CD88, CD123, CD125, CD154, CCR2, CD203c, IL-18R, TLR2, TLR4, TLR6, CRTH2, FceR1, CD3, CD4, CD7, CD15, CD36, CD45RA, HLA-DR, CD235a, CD19, CD34, or those described in e.g., US2010/0167317, the contents of which are incorporated herein by reference in its entirety. Examples of intracellular basophil markers include CD281, CD289, C-EBP alpha, and/or GATA2.

In certain embodiments, the TIM-3 inhibitor is targeted to eosinophils, for example, using eosinophil cell surface markers such as CD9, CD15, CD24, CD35, CD43, CD64, CD116, CD123, CD125, CD126, CD170, CD193, CD244, and/or FceR1.

TABLE
Exemplary sequences for myeloid cell-specific markers

| Myeloid Cell | | |
|---|---|---|
| Marker | Myeloid Cell | Exemplary Protein Sequence |
| CD45 | Neutrophils, | mtmylwlkll afgfafldte vfvtgqsptp sptglttakm psvplssdpl pthttafspa |
| (NCBI Accession | monocytes, | stferendfs etttslspdn tstqvspdsl dnasafnttg vssvqtphlp thadsqtpsa |
| No: P08575.3) | macrophages, | gtdtqtfsgs aanaklnptp gsnaisdvpg erstastfpt dpvspltttl slahhssaal |
| | T cells, B | partsnttit antsdaylna setttlspsg savistttia ttpskptcde kyanitvdyl |
| | cells, | ynketklfta klnvnenvec gnntctnnev hnltecknas vsishnscta pdktlildvp |
| | NK cells, | pgvekfqlhd ctqvekadtt iclkwkniet ftcdtqnity rfqcgnmifd nkeiklenle |
| | DC1, DC2, | peheykcdse ilynnhkftn askiiktdfg spgepqiifc rseaahqgvi twnppqrsfh |
| | Mig DC DC1 | nftlcyiket ekdclnldkn likydlqnlk pytkyvlslh ayiiakvqrn gsaamchftt |
| | | ksappsqvwn mtvsmtsdns mhvkcrpprd rngpheryhl eveagntlvr neshkncdfr |
| | | vkdlqystdy tfkayfhngd ypgepfilhh stsynskali aflafliivt siallvvlyk |
| | | iydlhkkrsc nldeqqelve rddekqlmnv epihadille tykrkiadeg rlflaefqsi |
| | | prviskfpik earkpfnqnk nryvdilpyd ynrvelsein gdagsnyina syidgfkepr |
| | | kyiaaqgprd etvddfwrmi weqkatvivm vtrceegnrn kcaeywpsme egtrafgdvv |
| | | vkinqhkrcp dyiiqklniv nkkekatgre vthiqftswp dhgvpedphl llklrrrvna |
| | | fsnffsgpiv vhcsagvgrt gtyigidaml egleaenkvd vygyvvklrr qrclmvqvea |
| | | qyilihqalv eynqfgetev nlselhpylh nmkkrdppse pspleaefqr lpsyrswrtq |
| | | hignqeenks knrnsnvipy dynrvplkhe lemskesehd sdessdddsd seepskyina |
| | | sfimsywkpe vmiaaqgplk etiqdgwqmi fqrkvkvivm ltelkhgdqe icaqywgegk |
| | | qtygdievdl kdtdksstyt lrvfelrhsk rkdsrtvyqy qytnwsveql paepkelism |
| | | iqvvkqklpq knssegnkhh kstpllihcr dgsqqtgifc allnllesae teevvdifqv |
| | | vkalrkarpg mvstfeqyqf lydviastyp aqngqvkknn hqedkiefdn evdkvkqdan |
| | | cvnplgapek lpeakeqaeg septsgtegp ehsvngpasp alnqgs (SEQ ID NO: |
| | | 30) |
| CD11b | neutrophils, | malrvlllta ltlchgfnld tenamtfqen argfgqsvvq lqgsrvvvga pqeivaanqr |
| (NCBI Accession | monocytes, | gslyqcdyst gscepirlqv pveavnmslg lslaattspp qllacgptvh qtcsentyvk |
| No.: | macrophages, | glcflfgsnl rqqpqkfpea lrgcpqedsd iaflidgsgs iiphdfrrmk efvstvmeql |
| P11215.2) | DC2, Mig | kksktlfslm qyseefrihf tfkefqnnpn prslvkpitq llgrthtatg irkvvrelfn |
| | DC DC2 | itngarknaf kilvvitdge kfgdplgyed vipeadregv iryvigvgda frseksrqel |
| | | ntiaskpprd hvfqvnnfea lktiqnqlre kifaiegtqt gssssfehem sqegfsaait |
| | | sngpllstvg sydwaggvfl ytskekstfi nmtrvdsdmn daylgyaaai ilrnrvqslv |
| | | lgapryqhig lvamfrqntg mwesnanvkg tqigayfgas lcsvdvdsng stdlvligap |
| | | hyyeqtrggq vsvcplprgr arwqcdavly geqgqpwgrf gaaltvlgdv ngdkltdvai |
| | | gapgeednrg avylfhgtsg sgispshsqr iagsklsprl qyfgqslsgg qdltmdglvd |
| | | ltvgaqghvl llrsqpvlrv kaimefnpre varnvfecnd qvvkgkeage vrvclhvqks |
| | | trdrlregqi qsvvtydlal dsgrphsrav fnetknstrr qtqvlgltqt cetlklqlpn |
| | | ciedpvspiv lrlnfslvgt plsafgnlrp vlaedaqrlf talfpfeknc gndnicqddl |
| | | sitfsfmsld clvvggpref nvtvtvrndg edsyrtqvtf ffpldlsyrk vstlqnqrsq |
| | | rswrlacesa sstevsgalk stscsinhpi fpensevtfn itfdvdskas lgnklllkan |
| | | vtsennmprt nktefqlelp vkyavymvvt shgvstkyln ftasentsrv mqhqyqvsnl |
| | | gqrslpislv flvpvrlnqt viwdrpqvtf senlsstcht kerlpshsdf laelrkapvv |
| | | ncsiavcqri qcdipffgiq eefnatlkgn lsfdwyikts hnhllivsta eilfndsvft |
| | | llpgqgafvr sqtetkvepf evpnplpliv gssvggllll alitaalykl gffkrqykdm |
| | | mseggppgae pq (SEQ ID NO: 31) |
| Ly6G | neutrophils, | mdtchiaksc vlillvvllc aeraqglecy ncigvppets cntttopfsd gfcvaleiev |
| (NCBI Accession | monocytes | ivdshrskvk snlclpicpt tldnteitgn avnvktycck edlcnaavpt ggsswtmagv |
| No. | | llfslvsvll qtfl (SEQ ID NO: 32) |
| NP_001361159.1) | | |
| F4/80 | macrophages | mrgfnlllfw gccvmhsweg hirptrkpnt kgnncrdstl cpayatctnt vdsyycackq |
| (UniProtKB/Swiss- | | gflssngqnh fkdpgvrckd idecsqspqp cgpnsscknl sgrykcscld gfssptgndw |
| Prot: Q14246.3) | | vpgkpgnfsc tdinecltss vcpehsdcvn smgsyscscq vgfisrnstc edvdecadpr |
| | | acpehatcnn tvgnyscfcn pgfesssghl sfqglkasce didectemcp instctntpg |
| | | syfctchpgf apsngqlnft dqgvecrdid ecrqdpstcg pnsictnalg syscgciagf |
| | | hpnpegsqkd gnfscqrvlf kckedvipdn kqiqqcqegt avkpayvsfc aqinnifsvl |
| | | dkvcenkttv vslknttesf vpvlkqistw tkftkeetss latvflesve smtlasfwkp |
| | | sanitpavrt eyldieskvi nkecseenvt ldlvakgdkm kigcstiees estettgvaf |
| | | vsfvgmesvl nerffkdhqa plttseiklk mnsrvvggim tgekkdgfsd piiytleniq |
| | | pkqkferpic vswstdvkgg rwtsfgcvil easetytics cnqmanlavi masgeltmdf |
| | | slyiishvgi iislvclvla iatfllcrsi rnhntylhlh lcvclllakt lflagihktd |
| | | nkmgcaiiag flhylflacf fwmlveavil flmvrnlkvv nyfssrnikm lhicafgygl |
| | | pmlvvvisas vqpqgygmhn rcwlntetgf iwsflgpvct vivinslllt wtlwilrqrl |
| | | ssvnaevstl kdtrlltfka faqlfilgcs wvlgifqigp vagvmaylft iinslqgafi |
| | | flihcllngq vreeykrwit gktkpssqsq tsrillssmp sasktg (SEQ ID NO: |
| | | 33) |
| CD3 | macrophages | mehstflsgl vlatllsqvs pfkipieele drvfvncnts itwvegtvgt llsditrldl |
| (delta chain) | | gkrildprgi yrcngtdiyk dkestvqvhy rmcqscveld patvagiivt dviatlllal |
| (NCBI Accession | | gvfcfaghet grlsgaadtq allrndqvyq plrdrddaqy shlggnwarn k (SEQ ID |
| Nos.: | | NO: 34) |
| NP_000723.1, | | |
| NP_001035741.1, | | |
| P04234.1) | | |
| CD3 (epsilon | macrophages | mqsgthwrvl glcllsvgvw gqdgneemgg itqtpykvsi sgttviltcp qypgseilwq |
| chain) | | hndkniggde ddknigsded hlslkefsel eqsgyyvcyp rgskpedanf ylylrarvce |
| (NCBI Accession | | ncmemdvmsv ativivdici tggllllvyy wsknrkakak pvtrgagagg rqrgqnkerp |
| No.: P07766.2) | | ppvpnpdyep irkgqrdlys glnqrri (SEQ ID NO: 35) |
| CD3 (gamma | macrophages | meqgkglavl ilaiillqgt laqsikgnhl vkvydyqedg svlltcdaea knitwfkdgk |
| chain) | | migfltedkk kwnlgsnakd prgmyqckgs qnkskplqvy yrmcqnciel naatisgflf |
| (NCBI Accession | | aeivsifvla vgvyfiagqd gvrqsrasdk qtllpndqly qplkdreddq yshlqgnqlr |
| Nos.: P09693.1) | | rn (SEQ ID NO: 36) |
| CD4 | macrophages | mnrgvpfrhl llvlqlallp aatqgkkvvl gkkgdtvelt ctasqkksiq fhwknsnqik |
| (NCBI Accession | | ilgnqgsflt kgpsklndra dsrrslwdqg nfpliiknlk iedsdtyice vedqkeevql |
| No. QDC22486.1) | | lvfgltansd thllqgqslt ltlesppgss psvqcrsprg kniqggktls vsqlelqdsg |
| | | twtctvlqnq kkvefkidiv vlafqkassi vykkegeqve fsfplaftve kltgsgelww |
| | | qaerasssks witfdlknke vsvkrvtqdp klqmgkklpl hltlpqalpq yagsgnltla |
| | | leaktgklhq evnlvvmrat qlqknltcev wgptspklml slklenkeak vskrekavwv |
| | | lnpeagmwqc llsdsgqvll esnikvlptw stpvqpmali vlggvaglll figlgiffcv |
| | | rcrhrrrqae rmsqikrlls ekktcqcphr fqktcspi (SEQ ID NO: 37) |
| CD8 | macrophages | malpvtalll plalllhaar psqfrvspld rtwnlgetve lkcqvllsnp tsgcswlfqp |
| (NCBI Accession | | rgaaasptfl lylsqnkpka aegldtqrfs gkrlgdtfvl tlsdfrrene gyyfcsalsn |
| No. | | simyfshfvp vflpakpttt paprpptpap tiasqplslr peacrpaagg avhtrgldfa |
| NP_001139345.1, | | cdiyiwapla gtcgvlllsl vitlycnhrn rrrvckcprp vvksgdkpsl saryv (SEQ |
| NP_741969.1) | | ID NO: 38) |
| CD19 | B cells | mppprllffl lfltpmevrp eeplvvkvee gdnavlqclk gtsdgptqql twsresplkp |
| (NCBI Accession | | lklslglpg lgihmrplas wlfifnvsqq mggfylcqpg ppsekawqpg wtvnvegsge |
| No. AAA69966.1) | | lfrwnvsdlg glgcglknrs segpsspsgk lmspklyvwa kdrpeiwege ppcvpprdsl |
| | | nqslsqdltm apgstlwlsc gvppdsvsrg plswthvhpk gpksllslel kddrpardmw |
| | | vmetglllpr ataqdagkyy chrgnltmsf hleitarpvl whwllrtggw kvsavtlayl |
| | | ifclcslvgi lhlqralvlr rkrkrmtdpt rrffkvtppp gsgpqnqygn vlslptptsg |
| | | lgraqrwaag lggtapsygn pssdvqadga lgsrsppgvg peeeegegye epdseedsef |
| | | yendsnlgqd qlsqdgsgye npedeplgpe dedsfsnaes yenedeeltq pvartmdfls |
| | | phgsawdpsr eatslgsqsy edmrgilyaa pqlrsirgqp gpnheedads yenmdnpdgp |
| | | dpawggggrm gtwstr (SEQ ID NO: 39) |
| B220 | B cells | mylwlkllaf gfafldtevf vtgqsptpsp tglttakmps vplssdplpt httafspast |
| (NCBI Accession | | ferendfset ttslspdnts tqvspdsldn asafnttgvs svqtphlpth adsqtpsagt |
| No. AAH14239.1) | | dtqtfsgsaa naklnptpgs naisdaylna setttlspsg savistttia ttpskptcde |
| | | kyanitvdyl ynketklfta klnvnenvec gnntctnnev hnltecknas vsishnscta |
| | | pdktlildvp pgvekfqlhd ctqvekadtt iclkwkniet ftcdtqnity rfqcgnmifd |
| | | nkeiklenle peheykcdse ilynnhkftn askiiktdfg spgepqiifc rseaahqgvi |
| | | twnppqrsfh nftlcyiket ekdclnldkn likydlqnlk pytkyvlslh ayiiakvqrn |
| | | gsaamchftt ksappsqvwn mtvsmtsdnsmhvkcrpprd rngpheryhl eveagntlvr |
| | | neshkncdfr vkdlqystdy tfkayfhngd ypgepfilhh stsynskali aflafliivt |
| | | siallvvlyk iydlhkkrsc nldeqqelve rddekqlmnv epihadille tykrkiadeg |
| | | rlflaefqsi prviskfpik earkpfnqnk nryvdilpyd ynrvelsein gdagsnyina |
| | | syidgfkepr kyiaaqgprd etvddfwrmi weqkatvivm vtrceegnrn kcaeywpsme |
| | | egtrafgdvv vkinqhkrcp dyiiqklniv nkkkkk (SEQ ID NO: 40) |
| NK1.1 | NK cells | msasgpeapg dipalppppq pgsgpappap aaaaqeamdg raelpafpra gapplaasdt |
| (NCBI Accession | | vpaapegaga arpaaplrpt sfsvldildp nkfnsrrrrc vllgpvapaa capcasapca |
| No. | | papaasgrpp raeelerral agaggvgaag aeppnagdpf kageaetndt ngyssggggh |
| NP_001277008.1 | | spsadsgdev pddedddede apeteaarga eeargggggl gargsgcqga aetdaspgat |
| | | vdeaaapgpr enspvaqgpp ggaaapggag ttpqgtataa kpkrkrtgsd sksgkprrar |
| | | taftyeqlva lenkfkatry lsvcerlnla lslsltetqv kiwfqnrrtk wkkqnpgadt |
| | | saptgggggp gpgagpgtgl pgglsplsps ppmgaplgmh gpagypahgp gglvcaaqlp |
| | | flsspavlsp fvlgsqtyga pafyaphl (SEQ ID NO: 41) |
| MHC Class II | DC1, DC2, | mhrrrsrscr edqkpvmddq rdlisnneql pmlgrrpgap eskcsrgaly tgfsilvtll |
| (UniProtKB/Swiss- | Mig DC DC1 | lagqattayf lyqqqgrldk ltvtsqnlql enlrmklpkp pkpvskmrma tpllmqalpm |
| Prot: P04233.3) | | galpqgpmqn atkygnmted hvmhllqnad plkvypplkg sfpenlrhlk ntmetidwkv |
| | | feswmhhwll femsrhsleq kptdappkvl tkcqeevshi pavhpgsfrp kcdengnylp |
| | | lqcygsigyc wcvfpngtev pntrsrghhn csesleledp ssglgvtkqd lgpvpm |
| | | (SEQ ID NO: 42) |
| CD11c | DC1, DC2, | mtrtraalll ftalatslgf nldteeltaf rvdsagfgds vvqyanswvv vgapqkitaa |
| (NCBI Accession | Mig DC DC1 | nqtgglyqcg ystgacepig lqvppeavnm slglslastt spsqllacgp tvhhecgrnm |
| No. P20702.3) | | yltglcfllg ptqltqrlpv srqecprqeq divflidgsg sissrnfatm mnfvravisq |
| | | fqrpstqfsl mqfsnkfqth ftfeefrrss nplsllasvh qlqgftytat aiqnvvhrlf |
| | | hasygarrda akilivitdg kkegdsldyk dvipmadaag iiryaigvgl afqnrnswke |
| | | lndiaskpsq ehifkvedfd alkdiqnqlk ekifaiegte ttssssfele magegfsavf |
| | | tpdgpvlgav gsftwsggaf lyppnmsptf inmsqenvdm rdsylgyste lalwkgvqsl |
| | | vlgapryqht gkaviftqvs rqwrmkaevt gtqigsyfga slcsvdvdsd gstdlvliga |
| | | phyyeqtrgg qvsvcplprg wrrwwcdavl ygeqghpwgr fgaaltvlgd vngdkltdvv |
| | | igapgeeenr gavylfhgvl gpsispshsq riagsqlssr lqyfgqalsg gqdltqdglv |
| | | dlavgargqv lllrtrpvlw vgvsmqfipa eiprsafecr eqvvseqtlv qsniclyidk |
| | | rsknllgsrd lqssvtldla ldpgrlspra tfqetknrsl srvrvlglka hcenfnlllp |
| | | scvedsvtpi tlrlnftlvg kpllafrnlr pmlaadaqry ftaslpfekn cgadhicqdn |
| | | lgisfsfpgl ksllvgsnle lnaevmvwnd gedsygttit fshpaglsyr yvaegqkqgq |
| | | lrslhltcds apvgsqgtws tscrinhlif rggaqitfla tfdvspkavl gdrllltanv |
| | | ssenntprts kttfqlelpv kyavytvvss heqftkylnf seseekeshv amhryqvnnl |
| | | gqrdlpvsin fwvpvelnge avwmdvevsh pqnpslrcss ekiappasdf lahiqknpvl |
| | | dcsiagclrf rcdvpsfsvq eeldftlkgn lsfgwvrqil qkkvsvvsva eitfdtsvys |
| | | qlpgqeafmr aqtttvleky kvhnptpliv gssiggllll alitavlykv gffkrqykem |
| | | meeangqiap engtqtpspp sek (SEQ ID NO: 43) |
| CD103/XCR1 | DC1, Mig | mwlfhtllci aslallaafn vdvarpwltp kggapfvlss llhqdpstnq twllvtsprt |
| (NCBI Accession | DC DC1 | krtpgplhrc slvqdeilch pvehvpipkg rhrgvtvvrs hhgvliciqv lvrrphslss |
| No. AAI13437.1) | | eltgtcsllg pdlrpqaqan ffdlenlldp darvdtgdcy snkegggedd vntarqrral |
| | | ekeeeedkee eedeeeeeag teiaiildgs gsidppdfqr akdfisnmmr nfyekcfecn |
| | | falvqyggvi qtefdlrdsq dvmaslarvq nitqvgsvtk tasamqhvld siftsshgsr |
| | | rkaskvmvvl tdggifedpl nlttvinspk mqgverfaig vgeefksart arelnliasd |
| | | pdethafkvt nymaldglls klryniisme gtvgdalhyq laqigfsaqi lderqvllga |
| | | vgafdwsgga llydtrsrrg rflnqtaaaa adaeaaqysy lgyavavlhk tcslsyvaga |
| | | pqykhhgavf elqkegreas flpvlegeqm gsyfgselcp vdidmdgstd fllvaapfyh |
| | | vhgeegrvyv yrlseqdgsf slarilsghp gftnarfgfa maamgdlsqd kltdvaigap |
| | | legfgaddga sfgsvyiyng hwdglsasps qrirastvap glqyfgmsma ggfdisgdgl |
| | | aditvgtlgq avvfrsrpvv rlkvsmaftp salpigfngv vnvrlcfeis svttasesgl |
| | | reallnftld vdvgkqrrrl qcsdvrsclg clrewssgsq lcedlllmpt egelceedcf |
| | | snasvkvsyq lqtpegqtdh pqpildryte pfaifqlpye kacknklfcv aelqlattvs |
| | | qqelvvgltk eltlninltn sgedsymtsm alnyprnlql krmqkppspn iqcddpqpva |
| | | svlimncrig hpvlkrssah vsvvwqleen afpnrtadit vtvtnsnerr slanethtlq |
| | | frhgfvavls kpsimyvntg qglshhkefl fhvhgenlfg aeyqlqicvp tklrglqvaa |
| | | vkkltrtqas tvctwsqera cayssvqhve ewhsvscvia sdkenvtvaa eiswdhseel |
| | | lkdvtelqil geisfnksly eglnaenhrt kitvvflkde kyhslpiiik gsvggllvli |
| | | vilvilfkcg ffkrkyqqln lesirkaqlk senlleeen (SEQ ID NO: 44) |

### Antibody Inhibitors

As discussed above, in some embodiments, an inhibitory agent can comprise an antibody or antigen-binding fragment thereof that binds TIM-3 and inhibits TIM-3 activity. As used herein, the term “antibody reagent” is a polypeptide that includes at least one antigen-binding immunoglobulin variable domain sequence, and which specifically binds a given antigen (e.g., TIM-3).

In one embodiment, an inhibitory antibody or antigen binding fragment thereof binds the TIM-3 extracellular domain and inhibits binding of natural ligands to the TIM-3 receptor molecule. Other mechanisms, such as interference with receptor interaction with other (co)regulatory molecules can also be effective; the key is that binding of the antibody reagent inhibits receptor signaling, and this can be verified in an appropriate cell culture assay.

A variety of suitable antibody reagent formats are known in the art, such as complete antibodies, e.g., an IgG, or modified forms or fragments of such antibodies, including, as non-limiting examples, single chain antibodies, heterodimers of antibody heavy chains and/or light chains, an Fv fragment (e.g., single chain Fv (scFv), a disulfide bonded Fv), a Fab fragment, a Fab′ fragment, a F(ab′)2 fragment), a single variable domain (e.g., VH, VL, VHH), a dAb, and modified versions of any of the foregoing (e.g., modified by the covalent attachment of polyalkylene glycol (e.g., polyethylene glycol, polypropylene glycol, polybutylene glycol) or other suitable polymer). Antibody reagents or constructs can, if desired, be linked to an antibody Fc region, comprising one or both of CH2 and CH3 domains, and optionally, a hinge region. Such linkage can provide benefits such as increased serum half-life or promotion of effector function(s). Alternatively, antibody reagents or constructs can be fused to a carrier such as serum albumin to promote increased serum half-life.

In some embodiments, a polypeptide agent, including an antibody reagent, can be formatted as a bispecific polypeptide agent as described herein, and in US 2010/0081796 and US 2010/0021473, the contents of which are herein incorporated in their entireties by reference. Bispecific agents can include, for example, agents including separate binding sites specific for TIM-3 and a myeloma cell-specific cell surface marker. In other embodiments, a polypeptide agent, including an antibody reagent, can be formatted as a multispecific polypeptide agent, for example as described in WO 03/002609, the entire teachings of which are incorporated herein by reference.

Antibodies suitable for practicing the methods described herein are preferably monoclonal, and can include, but are not limited to, human, humanized or chimeric antibodies, including single chain antibodies, Fab fragments, F(ab′) fragments, fragments produced by a Fab expression library, and/or binding fragments of any of the above. Antibody reagents also include immunoglobulin molecules and immunologically active portions of immunoglobulin molecules, i.e., molecules that contain at least one, at least two, at least three or more antigen binding sites that specifically bind TIM-3 and one or more myeloid cell markers. Such immunoglobulin molecules can be of any type (e.g., IgG, IgE, IgM, IgD, IgA and IgY), class (e.g., IgG1, IgG2, IgG3, IgG4, IgAl and IgA2) or subclass of immunoglobulin molecule, as understood by one of skill in the art.

Additional types of antibodies include, but are not limited to, chimeric, humanized, and human antibodies. For application in man, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

The term “monoclonal antibody” as used herein refers to a population of antibodies that comprise an identical antigen-binding domain. In some embodiments, a monoclonal antibody can be produced by a single B cell clone, B cell hybrodima or its equivalent. Such a cell produces only one antibody, such that all antibodies produced by such a clone have the same antigen-binding domain. In contrast to polyclonal antibody preparations that typically include different antibodies directed against different determinants (epitopes) on a given target antigen, each monoclonal antibody is directed against a single determinant on the antigen. The term “monoclonal antibody” as used herein is not limited to antibodies produced through hybridoma technology. It is to be understood that the term “monoclonal antibody” refers to an antibody that is derived from a single clone, including any eukaryotic, prokaryotic or phage clone, and not the method by which the antibody is produced. For example, the monoclonal antibodies to be used in accordance with the methods and compositions described herein can be made by the hybridoma method first described by Kohler et al., Nature 256:495 (1975), or may be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example. A wide variety of methods for producing constructs with the antigen-binding domain of monoclonal antibodies are known to those of ordinary skill in the art.

Bispecific and multispecific polypeptide agents can comprise immunoglobulin variable domains that have different binding specificities. Such bispecific and multispecific polypeptide agents can comprise combinations of heavy and light chain domains. For example, a VH domain and a VL domain, which can be linked together in the form of an scFv (e.g., using a suitable linker such as (Gly4Ser)n (where n=1-8, e.g., 2, 3, 4, 5, 6, or 7) (SEQ ID NO: 45) can provide one antigen-binding domain that binds one or each target of a multispecific polypeptide agent e.g. TIM-3 and at least one myeloid cell-specific target. A construct that includes, e.g., an scFv that binds TIM-3 and an scFv that binds a myeloid cell marker, is said to be bispecific for TIM-3 and the myeloid marker. Similar arrangements can be applied in the context of, e.g., a bispecific F(ab′)2 construct.

Single domain antibody constructs are also contemplated for the development of bispecific or multispecific reagents described herein. In some embodiments of the aspects described herein, the bispecific and multispecific polypeptide agents do not comprise complementary VH/VL pairs which form an antigen-binding site that binds to a single antigen or epitope co-operatively as found in conventional two chain antibodies. Instead, in some embodiments, the bispecific and multispecific polypeptide agents can comprise a domain, wherein the V domains each have different binding specificities, such that two different epitopes or antigens are specifically bound. Camelid antibodies for example comprise only VH domains, and can be used to generate bispecific constructs when modified to a humanized scaffold.

In addition, in some embodiments, bispecific and multispecific polypeptide agents comprise one or more CH or CL domains. A hinge region domain can also be included in some embodiments. Such combinations of domains can, for example, mimic natural antibodies, such as IgG or IgM, or fragments thereof, such as Fv, scFv, Fab or F(ab′) 2 molecules. Other structures, such as a single arm of an IgG molecule comprising VH, VL, CHI and CL domains, are also encompassed within the embodiments described herein. Alternatively, in another embodiment, a plurality of bispecific polypeptide agents is combined to form a multimer. An Fc domain that binds human FcRn can extend circulating half-life by directing internalized antibodies into the FcRn-mediated recycling/secretory pathway. As another alternative, fusion with serum albumin can also extend serum half-life.

It will be appreciated by one skilled in the art that the light and heavy variable regions of a bispecific or multispecific polypeptide agent produced according to the methods described herein can be on the same polypeptide chain, or alternatively, on different polypeptide chains. In the case where the variable regions are on different polypeptide chains, then they can be linked via a linker, generally a flexible linker (such as a polypeptide chain), a chemical linking group, or any other method known in the art.

In another aspect, bispecific antibodies having an IgG-like format are provided. Such formats have the conventional four chain structure of an IgG molecule (2 heavy chains and two light chains), in which one antigen-binding region (comprised of a VH and a VL domain) specifically binds TIM-3 and the other antigen-binding region (also comprised of a VH and a VL domain) specifically binds a myeloid cell-specific protein (e.g., a receptor). In some embodiments, each of the variable regions (2 VH regions and 2 VL regions) is replaced with a dAb or single chain variable domain. The dAb(s) or single chain variable domain(s) that are included in an IgG-like format can have the same specificity or different specificities. In some embodiments, the IgG-like format is tetravalent and can have two, three or four specificities. For example, the IgG-like format can be bispecific and comprise 3 dAbs that have the same specificity and another dAb that has a different specificity; bispecific and comprise two dAbs that have the same specificity and two dAbs that have a common but different specificity; trispecific and comprise first and second dAbs that have the same specificity, a third dAb with a different specificity and a fourth dAb with a different specificity from the first, second and third dAbs; or tetraspecific and comprise four dAbs that each have a different specificity. Antigen-binding fragments of IgG-like formats (e.g., Fab, F(ab′)2, Fab′, Fv, scFv) can be prepared as is known to one of skill in the art, and as described herein.

In some embodiments of the aspects described herein, antigen-binding fragments of antibodies can be combined and/or formatted into non-antibody multispecific polypeptide structures to form multivalent complexes, which bind target molecules having the same epitope, thereby providing superior avidity. For example, natural bacterial receptors such as SpA can be used as scaffolds for the grafting of CDRs to generate ligands which bind specifically to one or more epitopes. Details of this procedure are described in U.S. Pat. No. 5,831,012, the contents of which are herein incorporated by reference in their entirety. Other suitable scaffolds include those based on fibronectin and affibodies. Details of suitable procedures are described in WO 98/58965, herein incorporated by reference in its entirety. Exemplary scaffolds are described in van den Beuken et al., J. Mol. Biol. 310:591-601 (2001), and in WO 00/69907 (Medical Research Council), the contents of each of which are herein incorporated by reference in their entireties. As but one example, a scaffold can be based on the ring structure of bacterial GroEL or other chaperone polypeptides. In some embodiments, protein scaffolds can be combined.

In some embodiments of the aspects described herein, bispecific or multispecific polypeptide agents can be formatted as fusion proteins that contain a first antigen-binding domain that is fused directly to a second antigen-binding domain. If desired, in some embodiments, such a format can further comprise a half-life extending moiety. As noted above, an Fc domain that binds human FcRn can extend circulating half-life by directing internalized antibodies into the FcRn-mediated recycling secretory pathway. As another alternative, fusion with serum albumin can also extend serum half-life. The benefits of serum albumin binding can also be realized with an antigen-binding domain that binds serum albumin. For example, a multispecific polypeptide agent can comprise a first antigen-binding domain specific for TIM-3, that is fused to a myeloid cell specific antibody binding domain and an antigen-binding domain that binds serum albumin.

Generally, the orientation of the polypeptide domains that have a binding site with binding specificity for a target, and whether a bispecific or multispecific polypeptide agent comprises a linker, are a matter of design choice. However, some orientations, with or without linkers, can provide better binding characteristics than other orientations. All orientations are encompassed by the aspects and embodiments described herein, and bispecific or multispecific polypeptide agents that contain an orientation that provides desired binding characteristics can be easily identified by screening.

In some embodiments of the aspects described herein, an inhibitor targets TIM-3 and is targeted to a myeloid cell via a myeloid cell-specific marker in a bi-or multispecific format, in order to inhibit or reduce expression or activity of TIM-3 specifically in myeloid cells. As used herein, a “myeloid specific cell marker” refers to a molecule that is present on the cell surface of or intracellularly of a given myeloid cell and that is not expressed or is expressed minimally in other cell populations, such as T cells. Non-limiting examples of myeloid cell-specific markers useful in the compositions and methods described herein include: neutrophil markers (CD45, CD11b, and/or Ly6G), monocyte markers (CD45, CD11b, and/or Ly6C); CD11b⁺ macrophage markers (CD45, CD11b, and/or F4/80); T cells (CD45, CD3, CD4 and/or CD8); B cells (CD45, B220 and/or CD19); Natural Killer cells (NK) (CD45 and/or NK1.1); dendritic cells subset 1 (DC1) (CD45, ClassII, CD11c, and/or CD103/XCR1); dendritic cells subset 2 (DC2) (CD45, ClassII, CD11c, and/or CD11b); and migratory DCI dendritic cells (CD45, ClassII, CD11c, CD11b and/or CD103/XCR1).

In one embodiment, a bispecific or multispecific antibody reagent as described herein can utilize TIM-3 binding site sequences from monoclonal antibodies that specifically bind human TIM-3, including, but not limited to those obtained from, Miltenyi Biotec, BD Biosciences, Abcam, Novus Bio, RND Systems and the like. In some embodiments, a bispecific or multispecific antibody reagent as described herein can utilize TIM-3 binding site sequences from monoclonal antibodies that specifically bind human TIM-3, including clone F38-2E2 (Biolegend), or clone 344823 (R&D Systems). For example, an antigen binding site specific for TIM-3 having the CDRs of 5D12 (BD Biosciences or Sigma Aldrich). Exemplary TIM-3 antibodies are described in Monney, L. et al. Nature 415, 536-541 (2002). Anti-TIM-3 monoclonal antibodies and V_{H}/V_{L} and/or CDR sequences thereof are described in, e.g., US2017/240633, U.S. Pat. No. 10,550,181; US2021/016359; US2019/0375839; US2020/022392; US2018/0186881, the contents of each of which are incorporated herein by reference in their entirety. TIM-3-inhibiting monoclonal antibodies have been developed for therapeutic use. See, e.g., Acharya et al., J. Immnuother. Cancer 8: e000911 (2020), Rotte et al., Ann. Oncol. 29: 71-83 (2018). Examples include the following: MBG453 (Sabatolimab, Novartis Pharmaceuticals), an isotype IgG4 (S228P) monoclonal (see ClinicalTrials.gov identifier Nos. NCT02608268, NCT03066648 and NCT03946670 and Curigliano et al., Cancer Res. 79: CT183 (2019); Mach, Santoro, Kim et al., ESMO (2019); Borate et al., Blood 134: 570 (2019)); TSR-022 (Tesaro), an isotype IgG4 monoclonal (see ClinicalTrials.gov identifier Nos. NCT02817633 and NCT030680508 and BMC 32^{nd} annual meeting and Pre-Conference Programs of the Society for Immunotherapy of Cancer (SITC 2017), Part one, J. Immunother. Cancer 5: 86 (2017); BMC 33^{rd} Annual Meeting & Pre-Conference Programs of the Society for Immunotherapy of Cancer (SITC 2018), J. Immunother. Cancer. 6: 115 (2018); Murtaza et al., Eur. J. Cancer 69: S102 (2016); Rebuffet et al., SITC 2019); Sym023 (Symphogen A/S), see ClinicalTrials. gov identifier Nos. NCT03311412 and NCT03489343, and Lindsted, Cancer Res. 78: 5629 (2018); BGBA425 (BeiGene), an IgG1 (variant, engineered to remove FcγR binding) monoclonal (see ClinicalTrials.gov identifier No. NCT03744468 and Zhang, Cancer Res. 77: 2628 (2017)); R07121661 (Hoffmann-La Roche), a bispecific antibody construct (see ClinicalTrials.gov identified No. NCT03708328 and Klein et al., Methods 154: 21-31 (2019)); LY3321367 (Eli Lilly & Co.), seeClinicalTrials. gov identifier No. NCT03099109 and Harding et al., ASCO-SITC Clinical Immuno-Oncology Symposium 12 (2019); ICAGN02390 (Incyte Corp.), an isotype IgG1k (N297A) monoclonal (see ClinicalTrials.gov identifier NCT03652077 and Waight et al., Cancer Res. 78: 3825 (2018)); and BMS-986258 (Bristol-Myers Squibb), an isotype IgGI monoclonal (see ClinicalTrials.gov identifier No. NCT03446040). Further, Kikushige et al. describe the development of a neutralizing anti-TIM-3 monoclonal ATIK2a (Kikushige et al., Cell Stem Cell 7: 708-717 (2010).

In one embodiment, a bispecific or multispecific antibody reagent as described herein can utilize TIM-3 binding site sequences from sabatolimab.

In one embodiment, a bispecific or multispecific antibody reagent as described herein can utilize CD11b or CD11c binding site sequences from monoclonal antibodies that specifically bind human CD11b or CD11c, including, but not limited to those obtained from, Abcam, ProteinTech, ThermoFisher Scientific, Invitrogen, Novus Bio, RND Systems, Beckman Coulter, Miltenyi Biotec and the like. Non-limiting examples of commercially available monoclonal antibodies specific for CD11b include, but are not limited to Thermo Fisher/Invitrogen monoclonals M1/70 (catalogue No. 14-0112-82) , M1/70.15 (catalogue No. MA1-80091), C67F154 (catalogue No. 14-0196-82), ICRF44 (catalogue No. 14-0118-82), JU93-81 (catalogue No. MA5-32793), RM290 (catalogue No. MA5-27894), LM2/1 (catalogue No. BMS-104), VIM12 (catalogue No. CD11B00), 5C6 (catalogue No. MA5-16528), and MEM-174 (catalogue No. MA1-19004). Non-limiting examples of commercially available monoclonal antibodies specific for CD11c include, but are not limited to Thermo Fisher/Invitrogen monoclonals 3.9 (catalogue No. 14-0116-82), BU15 (catalogue No. MA1-82142), SI19-06 (catalogue No. MA5-32062), 118/A5 (catalogue No. 13-9761-82), FK24 (catalogue No. MA1-35070), CBR-p150/4G1 (catalogue No. MA5-16613), and ZM103 (catalogue No. Z2414MP).

In one embodiment, a bispecific or multispecific antibody reagent as described herein can utilize CD47 binding site sequences from monoclonal antibodies that specifically bind human CD47, including, but not limited to those obtained from, Bio X Cell, Santa Cruz Biotechnology, Abcam, ThermoFisher Scientific, Invitrogen, Novus Bio, and the like. Exemplary CD47 antibodies and CDRs thereof are known in the art or can be found in e.g., US2021/0070855; US2013/0142786; US2020/0247886; US2020/385465; US2016/0304609, US2021/0179716, the contents of each of which are incorporated herein by reference in their entirety.

In some embodiments of the aspects described herein, the binding sites of bispecific polypeptide agents, such as bispecific antibodies, are directed against a target's ligand interaction site. In other embodiments of the aspects described herein, the binding sites of the bispecific polypeptide agents are directed against a site on a target in the proximity of the ligand interaction site, in order to provide steric hindrance for the interaction of the target with its receptor or ligand. Preferably, the site against which antibody reagents or polypeptide agents as described herein are directed is such that binding of the target to its receptor or ligand is modulated, and in particular, inhibited or prevented.

Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually done by affinity chromatography steps, but the product yields are low. Similar procedures are disclosed in WO 93/08829, and in Traunecker et al, EMBO J., 10:3655-3659 (1991), herein incorporated by reference in their entireties.

According to another approach, described, for example in WO96/27011, herein incorporated by reference in its entirety, the interface between a pair of antibody molecules can be engineered to maximize the percentage of heterodimers which are recovered from recombinant cell culture. Such interfaces can comprise at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

In one aspect, the bispecific antibodies described herein include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies can be made using any convenient cross-linking methods. Suitable cross-linking agents are well known in the art, and are disclosed in U.S. Pat. No. 4,676,980, along with a number of cross-linking techniques. In one embodiment, the bispecific antibodies do not comprise a heteroconjugate.

Techniques for generating bispecific antibodies from antibody fragments have also been described in the literature. For example, bispecific antibodies can be prepared using chemical linkage. For example, Brennan et al., Science, 229: 81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. A bispecific antibody produced using this method can be used in any of the compositions and methods described herein.

Another option for production of bispecific or multispecific antibody reagents uses the dual variable domain immunoglobulin, or DvDIg approach. See, e.g., DiGiammarino et al., Meth. Mol. Biol. 899: 145-156 (2012). In this approach, each arm of the immunoglobulin molecule has two or more antigen-binding domains, which can be different, linked in tandem. The design has the benefit of providing bi- or multi-specificity without the problems generated by random assortment of differing light chains with differing heavy chains.

In some embodiments, bispecific antibodies for use in the compositions and methods described herein can be produced using any of the methods described in U.S. Patent Application No.: 20100233173; U.S. Patent Application No.: 20100105873; U.S. Patent Application No.: 20090155275; U.S. Patent Application No.: 20080071063; and U.S. Patent Application No.: 20060121042, the contents of each of which are herein incorporated in their entireties by reference. In some embodiments, a bispecific antibody specific for TIM-3 and a myeloid cell marker can be produced using any of the methods described in U.S. Patent Application No.: 20090175867 and U.S. Patent Application No.: 20110033483the contents of which are herein incorporated in their entireties by reference.

In some embodiments, bispecific antibodies can be made by the direct recovery of Fab′-SH fragments recombinantly expressed, e.g., in E. coli, and chemically coupled to form bispecific antibodies. Chemical conjugation is based on the use of homo- and heterobifunctional reagents with E-amino groups or hinge region thiol groups. Homobifunctional reagents such as 5,5′-Dithiobis(2-nitrobenzoic acid) (DNTB) generate disulfide bonds between the two Fabs, and 0-phenylenedimaleimide (O-PDM) generate thioether bonds between the two Fabs (Brenner et al., 1985, Glennie et al., 1987). Heterobifunctional reagents such as N-succinimidyl-3-(2-pyridylditio) propionate (SPDP) combine exposed amino groups of antibodies and Fab fragments, regardless of class or isotype (Van Dijk et al., 1989). For example, Shalaby et al., J Exp. Med, 175: 217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described, and can be used in the generation of bispecific antibodies. For example, bispecific antibodies have been produced using leucine zippers (Kostelny et al., J. Immunol, 148(5): 1547-1553 (1992)). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VH and VL domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (VH-CH1-VH-CH1) which form a pair of antigen binding regions. Linear antibodies can be bispecific or multispecific.

Antibodies useful in the present methods can be described or specified in terms of the particular CDRs they comprise. The compositions and methods described herein encompass the use of an antibody or derivative thereof comprising a heavy or light chain variable domain, where the variable domain comprises (a) a set of three CDRs, and (b) a set of four framework regions, and in which the antibody or antibody derivative thereof specifically binds TIM-3, and a myeloid cell-specific marker.

Also provided herein are chimeric antibody derivatives of the bispecific and mutispecific polypeptide agents, i.e., antibody molecules in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the selected antigens, on the surface of differentiated cells or tumor-specific cells. See, for example, Takeda et al., 1985, Nature 314:452; Cabilly et al., U.S. Pat. No. 4,816,567; Boss et al.; Tanaguchi et al., European Patent Publication EP171496; European Patent Publication 0173494, United Kingdom patent GB 2177096B).

The bispecific and multispecific polypeptide agents described herein can also include humanized antibody derivatives. Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, antibodies described herein include derivatives that are modified, i.e., by the covalent attachment of another type of molecule to the antibody that does not prevent the antibody from binding to its target. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

Bispecific or multispecific antibodies as described herein can be generated by any suitable method known in the art. Monoclonal and polyclonal antibodies against TIM-3 and myeloid cell-specific markers are known in the art. To the extent necessary, e.g., to generate antibodies with particular characteristics or epitope specificity, the skilled artisan can generate new monoclonal or polyclonal anti-TIM-3, and anti-myeloid cell-specific marker antibodies as discussed below or as known in the art. In other embodiments, the bispecific and multispecific antibodies and antigen-binding fragments thereof described herein can utilize TIM-3 binding site sequences or CDRs from monoclonal antibodies against human TIM-3, such as, human monoclonal anti-TIM-3 IgG2 antibodies (e.g., TIM-3 clone 344801 from RND Systems or recombinant human antibody EPR20767 from Abcam); or those obtained from, BD Biosciences, Biolegend, RND Systems, Abcam and the like.

Antibodies can be produced in bacteria, yeast, fungi, protozoa, insect cells, plants, or mammalian cells (see e.g., Frenzel et al. (2013) Front Immunol. 4: 217). A mammalian expression system is generally preferred for manufacturing most of therapeutic proteins, such as antibodies, as they require post-translational modifications. A variety of mammalian cell expression systems are now available for expression of antibodies, including but not limited to immortalized Chinese hamster ovary (CHO) cells, mouse myeloma (NSO), mouse L-cells, myeloma cell lines like J558L and Sp2/0, baby hamster kidney (BHK), or human embryo kidney (HEK-293).

As used herein, the term “Complementarity Determining Regions” (CDRs, i.e., CDR1, CDR2, and CDR3) refers to the amino acid residues of an antibody variable domain the presence of which are necessary for specific antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain). Likewise, “frameworks” (FWs) comprise amino acids 1-23 (FW1), 35-49 (FW2), 57-88 (FW3), and 98-107 (FW4) in the light chain variable domain and 1-30 (FW1), 36-49 (FW2), 66-94 (FW3), and 103-113 (FW4) in the heavy chain variable domain taking into account the Kabat numbering system (Kabat et al., Sequences of Proteins of Immunological Interest, 5th Ed. Public Health Service, National Institutes of Health, Bethesda, Md. (1987, 1991)).

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.Methods and computer programs for determining sequence similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

In some embodiments as described herein, an antibody reagent is specific for a target and/or marker described herein (e.g., that binds specifically to and inhibits TIM-3).

In certain embodiments, an antibody or antigen binding fragment that binds to a myeloid cell marker comprises the CDRs of known commercial antibodies (e.g., from Biolegend & BD).

Such commercial antibodies from which CDRs can be identified and then used in the preparation of a myeloid cell-targeted antibody or antibody fragment include, but are not limited to: CD45 (30-F11), CD4 (RM4-5), CD8a (53-6.7), B220 (RA3-6B2), NK1.1 (PK136), CD11b (M1/70), CD11c (N418), CD103 (P84), XCRI (ZET), CD64 (X54-5/7.1), CD86 (GL-1), PDL1 (10F.9G2), CD27 (LG.3A10), IL-7Ra (A7R34), CXCR5 (L138D7), CX3CR1 (SA011F11), CD5 (53-7.3), CD24 (M1/69), PDL2 (122), Lag3 (C9B7W), TIGIT (GIGD7), Tim3 (5D12), Ly6C (HK1.4), Ly6G (1A8), TCRb (H57-597), F4/80 (BM8), Ki67 (16A8), T-bet (4B10), TCF1 (C63D9).

### RNA Interference (RNAi)

Other approaches for inhibiting TIM-3 expression and/or activity include the use of RNA interference (RNAi), aptamers as well as the use of small molecules. For each of these, it is contemplated that targeting to one or more myeloid cells can be achieved, for example, by conjugating the inhibitor to an aptamer that binds a myeloid cell specific molecule (e.g., a cell surface myeloid marker). Alternatively, liposomes comprising the RNAi, aptamers or small-molecule can be designed to display myeloid cell-specific cell-surface binding; molecules, e.g. aptamer or antibody binding domains on their surface to target delivery to myeloid cells. The design and testing of RNAi molecules that inhibit the expression of TIM-3 are known to those skilled in the art. For example, RNAi molecules that inhibit TIM-3 can be obtained from commercial sources such as Santa Cruz Biotechnology (Dallas, Texas), ThermoFisher (Waltham, MA), Horizon Discovery/Dharmacon™ (Cambridge, UK), and Sigma-Aldrich (St. Louis, MO), among others.

The RNAi molecule can be siRNA, shRNA, endogenous microRNA (miRNA), or artificial miRNA. In one embodiment, an RNAi molecule as described herein effects inhibition of the expression and/or activity of a target, e.g. TIM-3. In some embodiments of any of the aspects, the RNAi molecule is an siRNA that inhibits TIM-3 activity and/or expression. RNAi sequences targeting TIM-3 are known in the art (see e.g., Cheng et al. Int J Clin Exp Pathol (2018) 11(3):1157-1166))

Exemplary human TIM-3 siRNA sequences

| SEQ ID NO | Sense Strand (5′-3′) |
|---|---|
| 5 | AAGGTCCTCAGAAGTGGAATA |
| 6 | AAGTGGAATACAGAGCGGAGG |
| 7 | AAGCCTCATCTCTTTGGCCAA |
| 8 | AAGGAGCCTGTCCTGTGTTTG |
| 9 | AACCTGAAGTTGGTCATCAAA |
| 10 | AACCATCAGAATAGGCATCTA |
| 11 | AATGGTATTCTCATAGCAAAG |
| 12 | AAATATCCACATTGGCCAATG |
| 13 | AACATCTATACCATTGAAGAG |
| 14 | AATATCCACATTGGCCAATGA |
| 15 | GGTCCTCAGAAGTGGAATACA |
| 16 | GGTCGGTCAGAATGCCTATCT |
| 17 | GGACTGATGAAAGGGATGTGA |
| 18 | GTCCCTGACCATAGAGAATGT |
| 19 | GATCCAAATCCCAGGCATAAT |
| 20 | GCCTCCCTGATATAAATCTAA |
| 21 | GCCAATGACTTACGGGACTCT |
| 22 | GCTGGCTCTGGCTCTTATCTT |
| 23 | GGATTGGCAAATGCAGTAGCA |
| 24 | GCAGAGGGAATTCGCTCAGAA |
| 25 | GAGGGAATTCGCTCAGAAGAA |
| 26 | GTGGAGGAGCCCAATGAGTAT |
| 27 | GAGCCTCCCTGATATAAATTT |
| 28 | GTGCTCAGGACTGATGAAATT |
| 29 | GGTCCTCAGAAGTGGAATATT |

One skilled in the art can design siRNA, shRNA, or miRNA to target TIM-3, e.g., using publically available design tools. siRNA, shRNA, or miRNA can be synthetically made or expressed from a vector. Commercial sources include companies such as Dharmacon (Lafayette, CO) and Sigma Aldrich (St. Louis, MO), among others.

In some embodiments, the RNAi molecule can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The nucleobases in the RNAi molecule can be chemically modified to enhance stability or other beneficial characteristics. The nucleic acids featured in the methods and compositions described herein can be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

In one embodiment, the TIM-3 inhibitor comprises an miRNA. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be encoded by a nucleic acid that is expressed in the cell, e.g., from naked DNA, or can be encoded by a nucleic acid that is contained within a vector.

The agent may result in gene silencing of the target gene (e.g., TIM-3), such as with an RNAi molecule (e.g. siRNA or miRNA). This entails a decrease in the TIM-3 mRNA level in a cell by at least about 5%, at least about 10%, at least about 20%, at least about 30%, at least about 40%, at least about 50%, at least about 60%, at least about 70%, at least about 80%, at least about 90%, at least about 95%, at least about 99%, or even about 100% (i.e., below detectable limits by standard mRNA assay detection methods) of the TIM-3 mRNA level found in the cell in the absence of a TIM-2 inhibitor. One skilled in the art will be able to readily assess whether the siRNA, shRNA, or miRNA effectively targets e.g., TIM-3, for downregulation, for example by transfecting the siRNA, shRNA, or miRNA into cultured cells and detecting the levels of a gene product (e.g., TIM-3) found within the cell via western-blotting.

Expression Vectors: The RNAi agent can be contained in or expressed by a desired vector. Many such vectors useful for transferring exogenous genes into target mammalian cells are available. As used herein, a vector can be viral or non-viral. The term “vector” encompasses any genetic element that is capable of replication when associated with the proper control elements and that can transfer gene sequences to cells. A vector can include, but is not limited to, an expression vector, a plasmid, phage, transposon, cosmid, artificial chromosome, virus, virion, etc.

An expression vector can direct expression of an RNA or polypeptide (e.g., a TIM-3 inhibitor) from nucleic acid sequences contained therein linked to transcriptional regulatory sequences on the vector. The sequences expressed will often, but not necessarily, be heterologous to the cell. An expression vector can comprise additional elements, for example, the expression vector may have two replication systems, thus allowing it to be maintained in two organisms, for example in human cells for expression and in a prokaryotic host for cloning and amplification. Expression refers to the cellular processes involved in producing RNA and/or proteins and as appropriate, secreting proteins, including where applicable, but not limited to, for example, transcription, transcript processing, translation and protein folding, modification and processing. “Expression products” include RNA transcribed from a gene and processing derivatives thereof, such as siRNA, shRNA, miRNA, etc., and polypeptides obtained by translation of mRNA transcribed from a gene or gene construct.

Vectors can be episomal, e.g. plasmids, virus-derived vectors such as cytomegalovirus, adenovirus, etc., or can be integrated into the target cell genome, through homologous recombination or random integration, e.g. for retrovirus-derived vectors such as MMLV, HIV-1, ALV, etc. In some embodiments, combinations of retroviruses and an appropriate packaging cell line may also find use, where the capsid proteins will be functional for infecting the target cells. Commonly used retroviral vectors are “defective”, i.e. unable to produce viral proteins required for productive infection. Replication of the vector requires growth in the packaging cell line.

Integrating vectors, such as retroviral vectors, lentiviral vectors, hybrid adenoviral vectors, and herpes simplex viral vector are specifically contemplated for use in the methods described herein. Alternatively, non-integrative vectors (e.g., non-integrative viral vectors) can be used and can eliminate the risks posed by integrative retroviruses, as they do not incorporate their genome into the host DNA. Non-limiting examples of non-integrating viral vectors include Epstein Barr oriP/Nuclear Antigen-1 (“EBNA1”) vector, RNA Sendai viral vector, or an F-deficient Sendai virus vector. Another example of a non-integrative vector is a minicircle vector. Minicircle vectors are circularized vectors in which the plasmid backbone has been released leaving only the eukaryotic promoter and cDNA(s) that are to be expressed.

Viral vectors can also be targeted, e.g. to a given myeloid cell by manipulating the viral capsid to comprise or display a ligand for a myeloid cell-specific cell-surface molecule as known in the art.

### CRISPR-Mediated Inhibition of TIM-3

Provided herein, in certain embodiments, is a method of reducing TIM-3 expression or activity in a myeloid cell comprising introducing into the cell (a) one or more DNA sequences encoding one or more guide RNAs (gRNAs) that are complementary to one or more target sequences in the TIM-3 gene and (b) a nucleic acid sequence encoding a Clustered Regularly Interspaced Short Palindromic Repeat (CRISPR)-associated endonuclease, whereby the one or more gRNAs hybridize to the TIM-3 gene and the CRISPR-associated endonuclease cleaves the TIM-3 gene, and wherein TIM-3 expression or activity is reduced in the cell relative to a cell in which the one or more DNA sequences encoding the one or more gRNAs and the nucleic acid sequence encoding the CRISPR-associated endonuclease are not introduced. In some embodiments, the one or more gRNAs are complementary to one or more target sequences in exon 1, 2, 3, 4, 5, 6, or 7, or spanning across two exons of the TIM-3 gene. In some embodiments, the one or more gRNAs comprise a trans-activated small RNA (tracrRNA) and a CRISPR RNA (crRNA). In some embodiments, the one or more gRNAs are one or more single guide RNAs. In some embodiments, the CRISPR-associated endonuclease is a class 2 CRISPR-associated endonuclease, and in some embodiments, the class 2 CRISPR-associated endonuclease is Cas9 or Cas12a.

As used herein, the term “Cas binding domain” refers to a nucleic acid element or domain within a nucleic acid sequence or polynucleotide sequence that, in an effective amount, will bind or have an affinity for one or a plurality of CRISPR-associated endonuclease (or functional fragments thereof). In some embodiments, in the presence of the one or a plurality of proteins (or functional fragments thereof) and a target sequence, the one or plurality of proteins and the nucleic acid element forms a biologically active CRISPR complex and/or can be enzymatically active on a target sequence.

In some embodiments, the Cas9 endonuclease can have a nucleotide sequence identical to the wild type Streptococcus pyogenes sequence. In some embodiments, the CRISPR-associated endonuclease can be a sequence from other species, for example other Streptococcusspecies, such as Streptococcus thermophilus; Pseudomona aeruginosa, Escherichia coli, or other sequenced bacteria genomes and archaea, or other prokaryotic microorganisms. In some embodiments, the CRISPR-associated endonuclease can be a Cas12a nuclease. The Cas12a nuclease can have a nucleotide sequence identical to a wild type Prevotella or Francisella sequence (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas12 endonucleases).

In some embodiments, the Cas binding domain comprises at least, or no more than, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially associates or binds to a biologically active CRISPR-associated endonuclease at a concentration and within a microenvironment suitable for CRISPR system formation.

In some embodiments, the guide RNA for use with the CRISPR Cas system can comprise a transcription terminator domain. The term “transcription terminator domain” refers to a nucleic acid element or domain within a nucleic acid sequence (or polynucleotide sequence) that, in an effective amount, prevents bacterial transcription when the CRISPR complex is in a bacterial species and/or creates a secondary structure that stabilizes the association of the nucleic acid sequence to one or a plurality of Cas proteins (or functional fragments thereof) such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of at least or no more than about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially drives association of the nucleic acid sequence (sgRNA, crRNA with tracrRNA, or other nucleic acid sequence) to a biologically active CRISPR complex at a concentration and microenvironment suitable for CRISPR complex formation.

Typically, a “CRISPR system” comprises transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus.

A “CRISPR target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is guanine and N is any naturally occurring nucleic acid. In some embodiments the target DNA comprises any one or combination of NNG, NNA, GAA, NNAGAAW and NGGNG, where G is guanine, A is adenine, and N is any naturally occurring nucleic acid

In some embodiments, a target sequence is located in the nucleus or cytoplasm of a cell.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), can also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient complementarity to be functional (bind the Cas protein or functional fragment thereof). In some embodiments, the tracr sequence has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a class 2 Cas9 or Cas12a protein. In some embodiments, the chemically synthesized guide sequence is used in conjunction with one or more vectors, wherein each vector comprises a coding sequence that encodes a CRISPR enzyme, such as a class 2 Cas9 or Cas12a protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, one or more additional guide sequence, tracr mate sequence, and tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid for a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

In some embodiments, a guide RNA can be a short, synthetic, chimeric tracrRNA/crRNA (a “single-guide RNA” or “sgRNA”). A guide RNA may also comprise two short, synthetic tracrRNA/crRNAs (a “dual-guide RNA” or “dgRNA”).

### Targeting a TIM-3 Inhibitor to Myeloid Cells

A TIM-3 inhibitor can be targeted to a myeloid cell or subset of myeloid cells using a variety of means known to those of skill in the art including, for example, fusion of a TIM-3 inhibitor to a targeting moiety; inclusion of a TIM-3 inhibitor in or on a nanoparticle, liposome or the like; or targeted conjugates of a TIM-3 inhibitor to a targeting moiety.

A myeloid cell-targeting moiety can include an antibody or antigen-binding fragment thereof (e.g., monoclonal, polyclonal, humanized, composite or chimeric antibody or fragment), a peptide, a polypeptide, a polymer, or a nanoparticle. In certain embodiments, the targeting moiety can comprise a binding pair, antibodies, monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments; and other targeting moieties include for example, aptamers, receptors, ligands, and fusion proteins.

In some embodiments, a TIM-3 inhibitor is targeted to myeloid cells using a composition comprising a “targeting particle,” which are substantially spherical bodies or membranous bodies from 500 nm-999 μm in size, such as e.g., liposomes, micelles, exosomes, microbubbles, or unilamellar vesicles. In some embodiments, the particle is less than 900 μm, less than 800 μm, less than 700 μm, less than 600 μm, less than 500 μm, less than 400 μm, less than 300 μm, less than 200 μm, less than 100 μm, less than 90 μm, less than 80 μm, less than 75 μm, less than 70 μm, less than 60 μm, less than 50 μm, less than 40 μm, less than 30 μm, less than 25 μm, less than 20 μm, less than 15 μm, less than 10 μm, less than 5 μm, less than 2 μm, less than 1 μm, less than 750 nm, less than 500 nm or smaller. As will be readily understood by those of skill in the art, a targeting particle that is of nanometer size (e.g., 10 to 1000 nm) is also referred to herein as a “nanoparticle.”

Nanoparticles are solid, colloidal particles consisting of macromolecular substances that vary in size from 10-1000 nanometers. A TIM-3 inhibitor can be dissolved, entrapped, adsorbed, attached or encapsulated into the nanoparticle matrix for targeting to a myeloid cell. The nanoparticle matrix can be comprised of biodegradable materials such as polymers or proteins. Depending on the method of preparation, nanoparticles can be obtained with different properties and release characteristics for the encapsulated therapeutic agents (Sahoo S K and Labhasetwar V, Nanotech approaches to drug delivery and imaging, DDT 8:1112-1120, 2003).

Nanoparticles, because of their small size, can penetrate through smaller capillaries and are taken up by cells, which allows efficient drug accumulation at the target sites (Panyam J et al., Nanoparticles can be made of biodegradable materials to permit sustained drug release within the target site over a period of days or even weeks. Nanoparticles can also be effective drug delivery mechanisms for drugs whose targets are cytoplasmic.

Targeted delivery of nanoparticles can be achieved by either passive or active targeting. Active targeting of a therapeutic agent is achieved by conjugating an TIM-3 inhibitor or the carrier system to a tissue or cell-specific ligand (Lamprecht et al., Biodegradable nanoparticles for targeted drug delivery in treatment of inflammatory bowel disease, J Pharmacol Exp Ther. 299:775-81, 2002). Passive targeting is achieved by coupling the therapeutic agent to a macromolecule that passively reaches the target organ or cell type (Monsky W L et al., Augmentation of transvascular transport of macromolecules and nanoparticles in tumors using vascular endothelial growth factor, Cancer Res. 59:4129-35, 1999). In one embodiment, dendritic cells are passively targeted on the basis of their natural tendency to take up nanoparticles. A TIM-3 inhibitor encapsulated in a nanoparticle or coupled to macromolecules such as high molecular weight polymers is contemplated for passive targeting of tumor tissue through the enhanced permeation and retention effect (Maeda H, The enhanced permeability and retention (EPR) effect in tumor vasculature: the key role of tumor-selective macromolecular drug targeting, Adv Enzyme Regul. 41:189-207, 2001; Sahoo S K et al., Pegylated zinc protoporphyrin: a water-soluble heme oxygenase inhibitor with tumor-targeting capacity, Bioconjugate Chem. 13:1031-8, 2002).

Nanoparticles prepared using biodegradable materials are preferable, however any suitable material can be used in the preparation of drug-delivery nanoparticles including, but not limited to, polymers, lipids (e.g., hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), or dimyristoylphosphatidylglycerol (DMPG)), metals (e.g., gold, silver, or a magnetic nanoparticle), etc. Representative, non-limiting examples of biodegradable polymers include synthetic polymers such as polymers of lactic acid and glycolic acid, polyanhydrides, poly(ortho)esters, polyurethanes, poly(hydroxybutiric acid), poly(valeric acid), and poly (lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin, and other hydrophilic proteins. The compositions described herein can also comprise bioerodible hydrogels which are prepared from materials and combinations of materials such as polyhyaluronic acids, casein, gelatin, glutin, polyanhydrides, polyacrylic acid, alginate, chitosan, poly(methyl methacrylates), poly(ethyl methacrylates), poly(butylmethacrylate), poly (isobutyl methacrylate), poly (hexylmethacrylate), poly (isodecyl methacrylate), poly (lauryl methacrylate), poly (phenyl methacrylate), poly(methyl acrylate), poly(isopropyl acrylate), poly(isobutyl acrylate), and poly(octadecyl acrylate). Preferred biodegradable polymers are polyglycolic acid, polylactic acid, copolymers of glycolic acid and L- or D,L-lactic acid, and copolymers of glycolide and L- or D,L-lactide.

In some embodiments, the targeted TIM-3 inhibitor comprises a polymer or a polymeric shell. The polymer can be natural or synthetic, with synthetic polymers being preferred due to the better characterization of degradation and, where appropriate, release profile of an incorporated agent. The polymer can be selected based on the period over which degradation or release of an agent is desired, generally in the range of at several weeks to several months, although shorter or longer periods may be desirable.

The compositions described herein can also include a conjugate of a lipid and a hydrophilic polymer, referred to as a ‘lipopolymer.’ Lipopolymers can be obtained commercially or can be synthesized using known procedures. For example, lipopolymers comprised of methoxy(polyethylene glycol) (mPEG) and a phosphatidylethanolamine (e.g., dimyristoyl phosphatidylethanolamine, dipalmitoyl phosphatidylethanolamine, 1,2-distearoyl-3-sn-glycerophosphoethanolamine (distearoyl phosphatidylethanolamine (DSPE)), or dioleoyl phosphatidylethanolamine) can be obtained from Avanti Polar Lipids, Inc. (Alabaster, Ala.) at various mPEG molecular weights (350, 550, 750, 1000, 2000, 3000, 5000 Daltons). Lipopolymers of mPEG-ceramide can also be purchased from Avanti Polar Lipids, Inc. Preparation of lipid-polymer conjugates are known in the art and are not described in detail herein.

The hydrophobic component of the lipopolymer can be virtually any hydrophobic compound having or modified to have a chemical group suitable for covalent attachment of a hydrophilic polymer chain. Exemplary chemical groups are, for example, an amine group, a hydroxyl group, an aldehyde group, and a carboxylic acid group. Preferred hydrophobic components are lipids, such as cholesterol, cholesterol derivatives, sphingomyelin, and phospholipids, such as phosphatidylcholine (PC), phosphatidylethanolamine (PE), phosphatidylglycerol (PG), phosphatidic acid (PA), phosphatidylinositol (PI), where the two hydrocarbon chains are typically between about 8-24 carbon atoms in length, and have varying degrees of unsaturation. These lipids are exemplary and are not intended to be limiting, as those of skill can readily identify other lipids that can be covalently modified with a hydrophilic polymer and incorporated into the particles described herein. In some embodiments, the lipopolymer is formed of polyethylene-glycol and a lipid, such as distearoyl phosphatidylethanolamine (DSPE), PEG-DSPE. PEG-DSPE has some degree of biodegradability in vivo, by virtue of the hydrolysable bonds between the fatty acids and the glycerol moiety.

Lipid nanoparticle formulations are specifically contemplated for delivering components of a CRISPR/Cas system. For example, the lipid nanoparticle formulations described in WO2017/0173054, the contents of which are incorporated herein by reference in its entirety, are contemplated for use with the methods and compositions described herein.

In some embodiments, a nanoparticle composition can further comprise a linker group, for example, between a polymer and a targeting moiety or the polymer and the TIM-3 inhibitor. In some embodiments the linker moiety is cleavable by an enzyme e.g., an esterase present in a target tissue or cell. In some embodiments, the linker moiety is cleavable by one type of esterase (e.g., a first type of an esterase present in a first cell type, e.g., a kidney cell) and is not cleavable by another type of esterase (e.g., a second type of esterase present in a second cell type, e.g., a cardiac cell). Thus, cleavage of the linker and activation of the drug and detectable moieties can be specific for a targeted cell type, such as a myeloid cell.

In one embodiment, the linker group comprises a functionalized phospholipid, such as a thiol-functionalized phospholipid, an amine functionalized phospholipid, or any combination thereof. In another embodiment, the amine-functionalized phospholipids can comprise DSPE-PEG(2000)Carboxylic Acid, DSPE-PEG(2000)Maleimide, DSPE-PEG(2000)PDP, DSPE-PEG(2000)Amine, DSPE-PEG(2000)Biotin, or any combination thereof. In another embodiment, the thiol-functionalized phospholipids can comprise phosphatidylthioethanol (PTE).

Exemplary myeloid cell-specific markers for targeting a nanoparticle and/or a TIM-3 inhibitor are described in the section entitled “Myeloid Cells and Markers Thereof.” Any marker or combination of markers discussed herein can be used to target a TIM-3 inhibitor using any of the means discussed above or known to those of skill in the art.

### Cancer

The methods and compositions provided herein can be used in the activation of inflammasomes in a subject having cancer, thereby treating the cancer. In some embodiments of these aspects and all such aspects described herein, the subject in need thereof has or has been diagnosed with cancer.

In certain embodiments, the cancer is metastatic or has the potential to be metastatic. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

In addition, cancers that can be treated using a myeloid-targeted TIM-3 inhibitor can be an angiogenic tumor.

Examples of cancer that can be treated with the methods and compositions provided herein include, but are not limited to, carcinoma, lymphoma, blastoma, sarcoma, and leukemia. More particular examples of such cancers include, but are not limited to basal cell carcinoma, biliary tract cancer; bladder cancer; bone cancer; brain and CNS cancer; breast cancer; cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), tumors of primitive origins and Meigs' syndrome.

In some embodiments described herein, the methods further comprise administering an anti-cancer therapy or agent to a subject in addition to the inhibitor of TIM-3 activity or expression in targeted myeloid cells. In some embodiments, inhibitors of TIM-3 are administered simultaneously, in the same or in separate compositions, or sequentially with the at least one additional anti-cancer therapy. For sequential administration, the TIM-3 inhibitor described herein can be administered first, and the additional agent can be administered second, or the order of administration can be reversed. The TIM-3 inhibitor and/or other therapeutic agents, procedures or modalities can be administered during periods of active disorder, or during a period of remission or less active disease. The agent can be administered before another treatment, concurrently with the treatment, post-treatment, or during remission of the disorder. In one embodiment, the TIM-3 inhibitor is administered prior to the onset of metastasis to prevent or reduce the degree of metastasis. In another embodiment, the TIM-3 inhibitor is administered following the detection of metastasis.

The term “anti-cancer therapy” refers to a therapy useful in treating cancer other than the TIM-3 targeting therapeutic disclosure disclosed herein. Examples of anti-cancer therapies include, but are not limited to, e.g., surgery, chemotherapeutic agents, growth inhibitory agents, cytotoxic agents, agents used in radiation therapy, anti-angiogenesis agents, apoptotic agents, anti-tubulin agents, and other agents to treat cancer, such as anti-HER2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVECTM (Imatinib Mesylate)), a COX2 inhibitor (e.g., celecoxib), interferons, cytokines, and other bioactive and organic chemical agents, etc. In some embodiments, an anti-cancer therapy can be one or more antagonists (e.g., neutralizing antibodies) that bind to one or more of the following targets: other TIM family members (e.g. TIM-1), CEACAM1 or any CEACAM family member, ErbB2, ErbB3, ErbB4, PDGFR-beta, BlyS, APRIL, BCMA or VEGF receptor(s), or TRAIL/Apo2. Combinations thereof are also specifically contemplated for the methods described herein.

An anti-cancer therapy can include a cytotoxic agent, such as a radioactive isotope (e.g. At²¹¹, I¹³¹, I¹²⁵, Y⁹⁰, Re¹⁸⁶, Re¹⁸⁸, Sm¹⁵³, Bi²¹², P³² or a radioactive isotope of Lu), chemotherapeutic agents, and toxins such as small molecule toxins or enzymatically active toxins of bacterial, fungal, plant or animal origin, including active fragments and/or variants thereof.

Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB.); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above. In addition, the methods of treatment can further include the use of radiation or radiation therapy.

Chemotherapeutic agents as used herein encompass both chemical and biological agents. These agents function, for example, to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; Baltzer L, Berkery R (eds): Oncology Pocket Guide to Chemotherapy, 2nd ed. St. Louis, Mosby-Year Book, 1995; Fischer D S (ed): The Cancer Chemotherapy Handbook, 4th ed. St. Louis, Mosby-Year Book, 2003)).

One of skill in the art will appreciate that the tumor cells themselves can, but need not express TIM-3.

By “reduce” or “inhibit” in terms of the cancer treatment methods described herein refers to a reduction in at least one parameter or symptom of a cancer by at least 20% at least 30%, at least 40%, at least 45%, at least 50%, at least 55%, at least 60%, at least 65%, at least 70%, at least 75% at least 80%, at least 85%, at least 90%, or at least 95% or greater. Parameters of symptoms of cancer that can be reduced or inhibited with the methods described herein include, but are not limited to, the presence or size of metastases or micrometastases, the size of the primary tumor, the presence or the size of the dormant tumor, tumor growth rate, pain, degree of angiogenesis in the tumor, etc. A patient or subject who is being treated for a cancer or tumor is one who a medical practitioner has diagnosed as having such a condition. Diagnosis can be by any suitable means.

### Dosage and Administration

The method and compositions provided herein enhance inflammasome activity and/or treat cancer in a subject by administering a therapeutically effective amount of a myeloid cell-targeted TIM-3 inhibitor. In one embodiment, the subject can be a mammal. In another embodiment, the mammal can be a human, although the approach is effective with respect to all mammals.

The appropriate dosage range for a myeloid cell-targeted TIM-3 inhibitor or a given anti-cancer agent depends upon the potency of the agent, and includes amounts large enough to produce the desired effect, e.g., increased activity of an inflammasome, or treatment of cancer. Although adverse side effects are often associated with anti-cancer agents, the dosage should not be so large as to cause unacceptable or life-threatening adverse side effects. Generally, the dosage will vary with the type of inhibitor, and with the age, condition, and sex of the patient. The dosage can be determined by one of skill in the art and can also be adjusted by the individual physician in the event of any complication.

The effective amount may be based upon, among other things, the size of the compound, the biodegradability of the compound, the bioactivity of the compound and the bioavailability of the compound. For example, if the compound does not degrade quickly, is bioavailable and highly active, a smaller amount will be required to be effective. One of skill in the art could routinely perform empirical activity tests for a compound to determine the bioactivity in bioassays and thus determine the effective amount.

Typically, the dosage ranges for a TIM-3 inhibitor (or an accompanying combination anti-cancer therapy) are in the range of 0.001 mg/kg body weight to 5 g/kg body weight. In some embodiments, the dosage range is from 0.001 mg/kg body weight to 1g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

As for when the compound, compositions and/or agent is to be administered, one skilled in the art can determine when to administer such compound and/or agent. The administration may be constant for a certain period of time or periodic and at specific intervals. The compound may be delivered hourly, daily, weekly, monthly, yearly (e.g. in a time release form) or as a one-time delivery. The delivery may be continuous delivery for a period of time, e.g. intravenous delivery. In one embodiment of the methods described herein, the agent is administered at least once per day. In one embodiment of the methods described herein, the agent is administered daily. In one embodiment of the methods described herein, the agent is administered every other day. In one embodiment of the methods described herein, the agent is administered every 6 to 8 days. In one embodiment of the methods described herein, the agent is administered weekly.

As one of skill in the art will appreciate, the dosage of a targeted TIM-3 inhibitor (or an adjunct anti-cancer therapy) can vary depending upon the dosage form employed and the route of administration utilized. Compositions, methods, and uses that exhibit large therapeutic indices (i.e., the dose ration between toxic and therapeutic effects) are preferred. A therapeutically effective dose can be estimated initially from cell culture assays. Also, a dose can be formulated in animal models to achieve a circulating plasma concentration range that includes the IC50, which achieves a half-maximal inhibition of measured function or activity as determined in cell culture, or in an appropriate animal model. The effects of any particular dosage can be monitored by a suitable bioassay. A therapeutically effective amount is an amount of an agent that is sufficient to produce a statistically significant, measurable change of a given symptom of a cancer (see “Efficacy Measurement” below). Such effective amounts can also be gauged in clinical trials as well as animal studies for a given agent.

An appropriate therapeutic amount or dose for treating a human subject can be informed by data collected in cell cultures or animal models. In some embodiments, the therapeutic efficacy can be estimated by the ED50 in an animal model (the dose therapeutically effective in 50% of the population) or in a cell cytotoxicity assay (where at least 50% of the cancer cells are killed).

Therapeutic compositions can be conventionally administered in a unit dose. The term “unit dose” when used in reference to a therapeutic composition refers to physically discrete units suitable as unitary dosage for the subject, each unit containing a predetermined quantity of an anti-cancer agent calculated to produce the desired therapeutic effect in association with the required physiologically acceptable diluent, i.e., carrier, or vehicle.

Administration of the doses recited above or as employed by a skilled clinician can be repeated for a limited and defined period of time. In some embodiments, the doses are given once a day, or multiple times a day (e.g., at least two times a day, at least three times a day etc). In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose. In one embodiment of the methods described herein, the agent is administered at least once per day. In one embodiment, the agent is administered daily. In one embodiment, the agent is administered every other day. In one embodiment, the agent is administered every 6 to 8 days. In one embodiment, the agent is administered weekly.

The agents described herein can be administered to a subject in need thereof by any appropriate route which results in an effective treatment in the subject. For example, agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired.

In some embodiments, the TIM-3 inhibitor described herein can be administered to a subject by any mode of administration that delivers the agent systemically or locally to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the agents described herein, other than directly into a target site, tissue, or organ, such that it enters the subject's circulatory system and, thus, is subject to metabolism and other like processes.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

Combination Therapies: In some embodiments, a targeted TIM-3 inhibitor as described herein is used in combination with at least one additional anti-cancer therapy, such as an anti-cancer agent or chemotherapeutic, X-rays, gamma rays or other sources of radiation to destroy cancer stem cells and/or cancer cells.

Combination therapy using an anti-TIM3 inhibitor and a second anti-cancer treatment (e.g., can comprise administration of the therapeutics to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the combination therapies can be administered at the same time or sequentially in any order at different points in time; however, if not administered at the same time, they should be administered sufficiently close in time so as to provide the desired therapeutic effect, preferably in a synergistic fashion. The combination cancer therapeutics (including a targeted TIM-3 inhibitor) can be administered separately, in any appropriate form and by any suitable route. When the combination therapies are not administered in the same pharmaceutical composition, it is understood that they can be administered in any order to a subject in need thereof. For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof.

When administered in combination, the anti-cancer agent or drug used in combination with a targeted TIM-3 inhibitor can be administered in an amount or dose that is lower (e.g., at least 20%, at least 30%, at least 40%, or at least 50% lower) or the same as the amount or dosage of the agent used individually, e.g., as a monotherapy.

Currently available anti-cancer therapies and their dosages, routes of administration and recommended usage are known in the art and have been described in such literature as the Physician's Desk Reference (60th ed., 2017).

### Compositions, Formulations and Packaging

Also provided herein are compositions, including pharmaceutical compositions, comprising a targeted TIM-3 inhibitor as described herein. In one embodiment, the compositions are pharmaceutical compositions. Pharmaceutical compositions for use with the methods described herein can be formulated in conventional manner using one or more physiologically acceptable carriers or excipients. Thus, the compounds and their physiologically acceptable salts and solvates can be formulated for administration by, for example, by aerosol, intravenous, oral or topical route. The compositions can be formulated for intralesional, intratumoral, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

In embodiments where the targeted TIM-3 inhibitor comprises an anti-TIM-3 RNAi, the RNAi can be mixed with a delivery system, such as a liposome system, and optionally can include an acceptable excipient. In a preferred embodiment, the composition is formulated for injection.

Techniques and formulations generally may be found in Remmington's Pharmaceutical Sciences, Meade Publishing Co., Easton, PA. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical composition can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., pharmaceutically acceptable oils, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use as described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator may be formulated containing a powder mix of the compound and a suitable powder base such as lactose or starch.

The targeted anti-TIM-3 inhibitors can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The TIM-3 inhibitors can also be formulated in rectal compositions such as suppositories or retention enemas, e.g., containing conventional suppository bases such as cocoa butter or other glycerides.

In addition to the formulations described previously, the TIM-3 inhibitors can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be through nasal sprays or using suppositories. For topical administration, the targeted TIM-3 inhibitors can be formulated into ointments, salves, gels, or creams as generally known in the art. A wash solution can be used locally to treat an injury or inflammation to accelerate healing.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

For therapies involving the administration of nucleic acids, such TIM-3 inhibitors can be formulated for a variety of modes of administration, including systemic and topical or localized administration. Techniques and formulations generally may be found in Remmington's Pharmaceutical Sciences, Meade Publishing Co., Easton, PA. For systemic administration, such as injection, RNAi comprising TIM-3 inhibitors can be formulated in liquid solutions, preferably in physiologically compatible buffers such as Hank's solution or Ringer's solution. In addition, the oligomers can be formulated in solid form and redissolved or suspended immediately prior to use. Lyophilized forms are also included.

### Efficacy

The efficacy of a given treatment for a cancer can be determined by the skilled clinician. However, a treatment is considered “effective treatment,” as the term is used herein, if any one or all of the signs or symptoms of the cancer is/are altered in a beneficial manner, or other clinically accepted symptoms or markers of disease are improved, or ameliorated, e.g., by at least 10% following treatment with an anti-cancer agent or combination thereof selected using the methods and assays described herein. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the cancer; or (2) relieving the disease, e.g., causing regression of symptoms; and (3) preventing or reducing the likelihood of the development of the disease, or preventing secondary diseases/disorders associated with the cancer (e.g., cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators of the disease, such as e.g., pain, tumor size, tumor growth rate, blood cell count, etc.

Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example animal models of cancer, e.g. a murine xenograft model. When using an experimental animal model, efficacy of treatment is evidenced when a statistically significant change in a marker is observed.

The invention may be as described in any one of the following numbered paragraphs:

1. A composition for selectively promoting inflammasome activity in myeloid cells, the composition comprising a TIM-3 inhibitor linked to an agent that specifically binds a myeloid cell surface marker.

2. The composition of paragraph 1, wherein the myeloid cell is a myeloid progenitor cell, a basophil, a neutrophil, an eosinophil, a monocyte, a macrophage, a dendritic cell, a granulocyte, a megakaryocyte or any combination thereof.

3. The composition of paragraph or paragraph 2, wherein the TIM-3 inhibitor specifically binds to TIM-3.

4. The composition of any one of paragraphs 1-3, wherein the TIM-3 inhibitor in the composition more efficiently promotes myeloid cell inflammasome activity than the TIM-3 inhibitor not linked to the agent that binds a myeloid cell surface marker.

5. The composition of any one of paragraphs 1-4, wherein the TIM-3 inhibitor in the composition more efficiently promotes tumor cell death than the TIM-3 inhibitor not linked to the agent that binds a myeloid cell surface marker.

6. The composition of paragraph 5, wherein the tumor cell death comprises pyroptosis.

7. The composition of any one of paragraphs 1-6, wherein the TIM-3 inhibitor comprises an antibody or antigen-binding fragment thereof that specifically binds TIM-3.

8. The composition of paragraph 7, wherein the antibody or antigen-binding fragment thereof binds an epitope on the extracellular domain of TIM-3.

9. The composition of any one of paragraphs 1-8, wherein the TIM-3 inhibitor promotes degradation of TIM-3 or RNA encoding TIM-3.

10. The composition of any one of paragraphs 1-6, wherein the TIM-3 inhibitor comprises an RNA interference (RNAi) molecule, an antisense molecule, or a small molecule.

11. The composition of any one of paragraphs 1-10, wherein the TIM-3 inhibitor is in or on a nanoparticle.

12. The composition of any one of paragraphs 1-11, wherein the myeloid cell surface marker is selected from CD47, CD11b and CD11c.

13. A pharmaceutical composition comprising the composition of any one of paragraphs 1-12 and a pharmaceutically-acceptable carrier.

14. A nanoparticle comprising a TIM-3 inhibitor in or on the nanoparticle.

15. The nanoparticle of paragraph 14, wherein the TIM-3 inhibitor comprises a nucleic acid, a peptide or a small molecule.

16. The nanoparticle of paragraph 14 or 15, wherein the TIM-3 inhibitor comprises an antibody or antigen-binding fragment thereof that specifically binds TIM-3.

17. The nanoparticle of paragraph 14 or 15, wherein the TIM-3 inhibitor comprises a nucleic acid that promotes degradation of RNA encoding TIM-3.

18. The nanoparticle of paragraph 17, wherein the nucleic acid is selected from an RNAi molecule, an miRNA, a CRISPR/Cas gRNA, and an antisense molecule.

19. The nanoparticle of any one of claims 14-18, which comprises a lipid nanoparticle.

20. The nanoparticle of any one of paragraphs 14-19, further comprising an agent that specifically binds to a myeloid cell surface marker.

21. A method of promoting inflammasome activity a myeloid cell, the method comprising contacting myeloid cell with a composition of any one of paragraphs 1-20.

22. The method of paragraph 21, wherein the inflammasome activity is induced to a greater extent than induced by a TIM-3 inhibitor lacking to an agent that specifically binds a myeloid cell surface marker.

23. The method of paragraph 21 or 22, wherein the myeloid cell is a myeloid progenitor cell, a basophil, a neutrophil, an eosinophil, a monocyte, a macrophage, a dendritic cell, a granulocyte, a megakaryocyte or any combination thereof.

24. The method of any one of paragraphs 21-23, wherein the myeloid cell is in a solid tumor microenvironment.

25. A method of promoting cancer cell death, the method comprising contacting a myeloid cell associated with the cancer cell with a composition of any one of paragraphs 1-20.

26. The method of paragraph 25, wherein inflammasome activity is induced to a greater extent than induced by a TIM-3 inhibitor lacking to an agent that specifically binds a myeloid cell surface marker.

27. The method of any one of paragraphs paragraph 21-26, wherein the myeloid cell is a myeloid progenitor cell, a basophil, a neutrophil, an eosinophil, a monocyte, a macrophage, a dendritic cell, a granulocyte, a megakaryocyte or any combination thereof.

28. The method of any one of paragraphs 25-27, wherein the cancer is acute myeloid leukemia (AML) or a solid tumor.

29. The method of any one of paragraphs 25-28, wherein the cancer cells do not express TIM-3.

30. A method of treating cancer, the method comprising administering a composition of any one of paragraphs 1-20 to a subject in need thereof.

31. The method of paragraph 30, wherein inflammasome activity in cancer-associated myeloid cells is induced to a greater extent than induced by a non-targeted TIM-3 inhibitor.

32. The method of paragraph 31, wherein the myeloid cell is a myeloid progenitor cell, a basophil, a neutrophil, an eosinophil, a monocyte, a macrophage, a dendritic cell, a granulocyte, a megakaryocyte or any combination thereof.

33. The method of any one of paragraphs 30-32, wherein the cancer is acute myeloid leukemia (AML), chronic myeloid leukemia (CML) or a solid tumor.

34. The method of any one of paragraphs 30-33, wherein cells of the cancer do not express TIM-3.

35. The method of any one of paragraphs 30-34, wherein death of cells of the cancer is induced to a greater extent than induced by a TIM-3 inhibitor that is not linked to an agent that specifically binds a myeloid cell surface marker.

36. The method of any one of paragraphs 30-35, wherein the cancer is a solid tumor.

37. The method of paragraph 36, wherein the microenvironment of the solid tumor is rendered less hostile to T cells by the administering.

38. The method of any one of paragraphs 30-37, wherein the cancer is metastatic.

39. The method of any one of paragraphs 30-38, wherein the cancer is angiogenic.

40. A composition comprising comprising a TIM-3 inhibitor linked to an agent that specifically binds a myeloid cell surface marker for use in promoting inflammasome activity or treating cancer in a subject.

41. The composition for use of paragraph 40, wherein the TIM-3 inhibitor specifically binds to TIM-3.

42. The composition for use of any one of paragraphs 40-41, wherein the TIM-3 inhibitor in the composition more efficiently promotes myeloid cell inflammasome activity than the TIM-3 inhibitor not linked to the agent that binds a myeloid cell surface marker.

43. The composition for use of any one of paragraphs 40-42, wherein the TIM-3 inhibitor in the composition more efficiently promotes tumor cell death than the TIM-3 inhibitor not linked to the agent that binds a myeloid cell surface marker.

44. The composition for use of any one of paragraphs 40-43, wherein the TIM-3 inhibitor comprises an antibody or antigen-binding fragment thereof that specifically binds TIM-3.

45. The composition for use of paragraph 44, wherein the antibody or antigen-binding fragment thereof binds an epitope on the extracellular domain of TIM-3.

46. The composition for use of any one of paragraphs 40-43, wherein the TIM-3 inhibitor promotes degradation of TIM-3 or RNA encoding TIM-3.

47. The composition for use of any one of paragraphs 40-46, wherein the TIM-3 inhibitor comprises an RNA interference (RNAi) molecule, an antisense molecule, or a small molecule.

48. The composition for use of any one of paragraphs 40-47, wherein the TIM-3 inhibitor is in or on a nanoparticle.

49. The composition for use of any one of paragraphs 40-48, wherein the myeloid cell surface marker is selected from CD47, CD11b and CD11c.

50. The composition for use of any one of paragraphs 40-49, further comprising a pharmaceutically acceptable carrier.

51. A composition comprising a TIM-3 inhibitor in or on the nanoparticle for use in promoting inflammasome activity or treating cancer in a subject.

### EXAMPLES

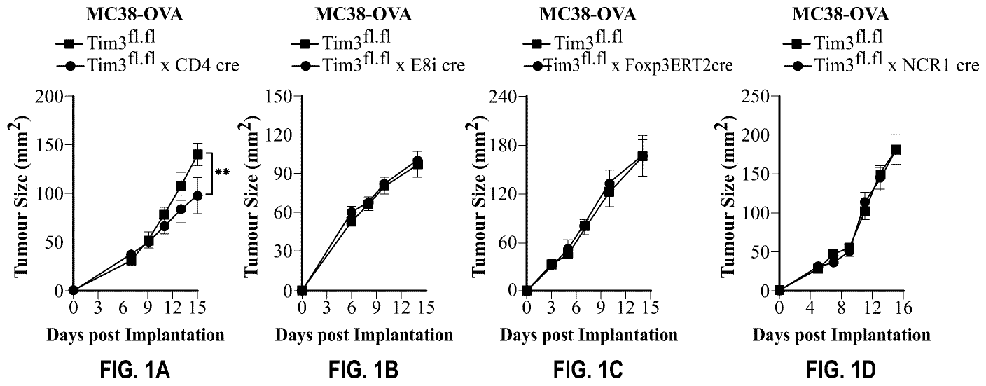

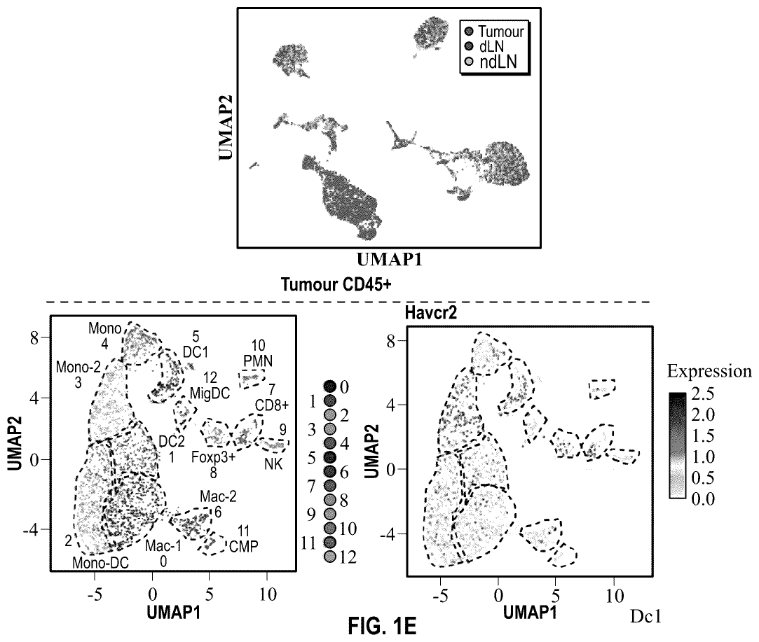

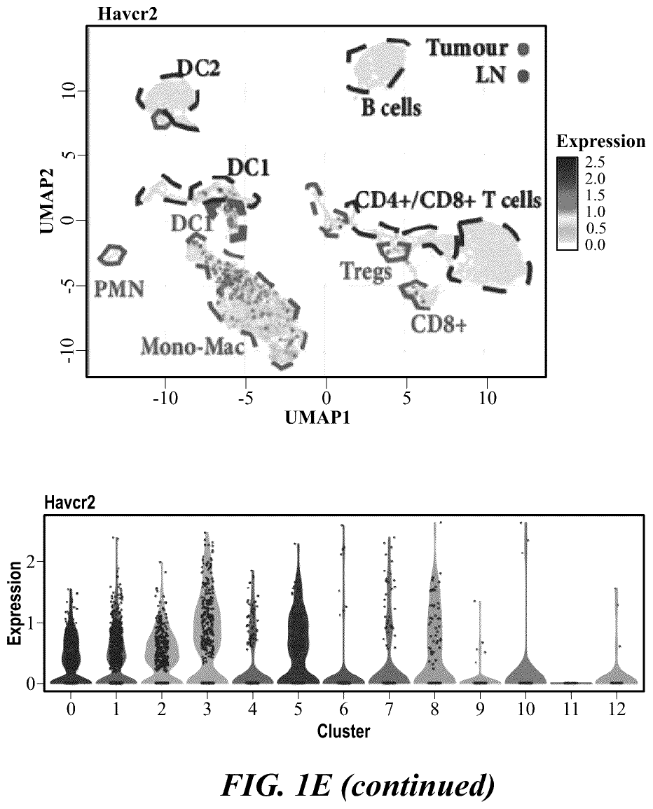

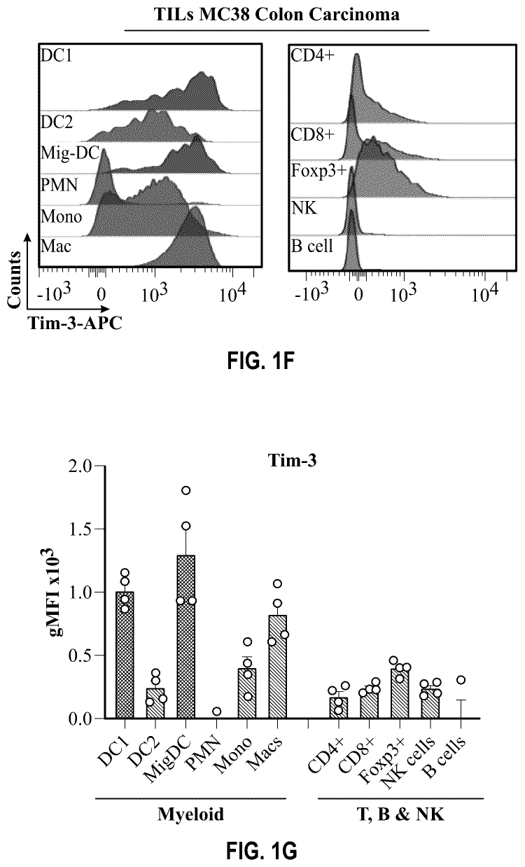

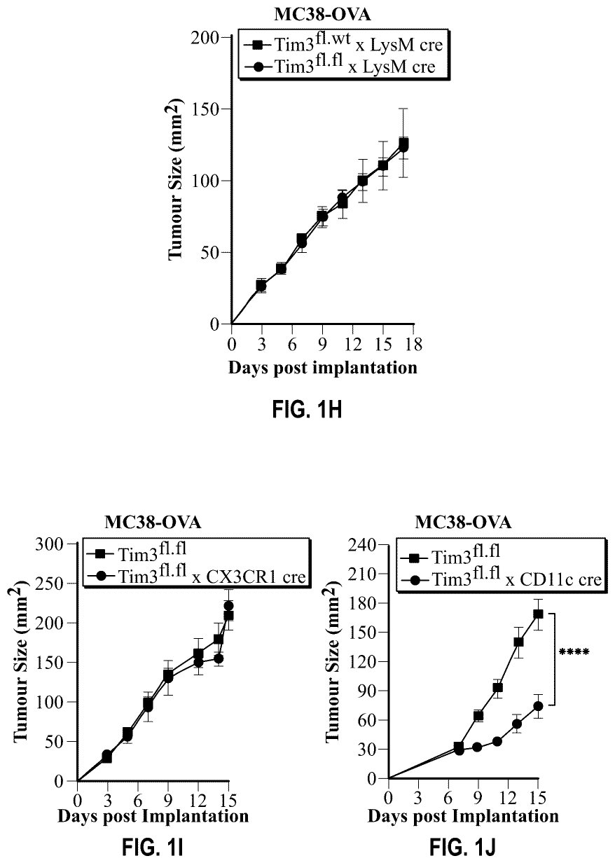

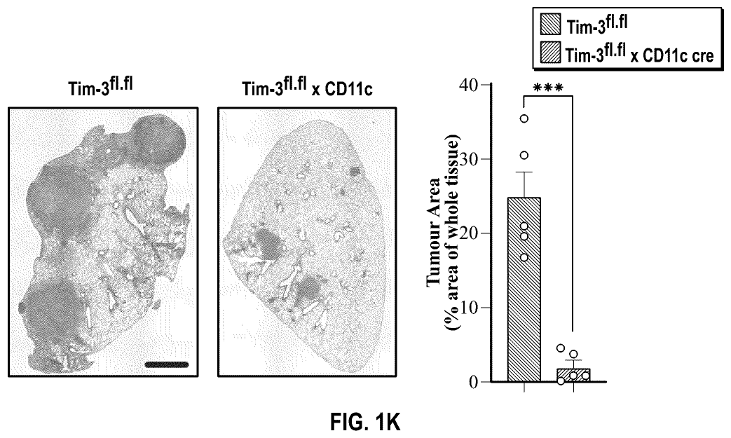

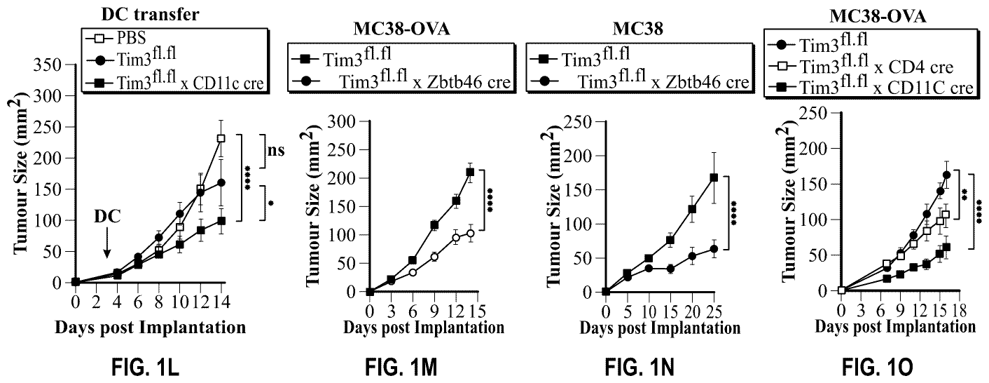

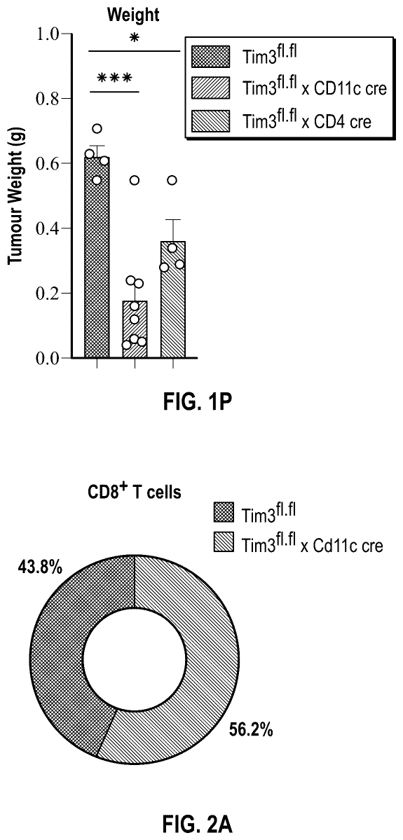

To address the specific function of Tim-3 within T cell populations, conditional knockout mice (Extended Data FIG. 1A) were generated deleting Tim-3 in (i) both CD4+ and CD8+ T cells, (ii) CD8+ T cells, or (iii) Foxp3+ regulatory T cells. Deletion in both CD4+ and CD8+ T cells (CD4cre) led to only a modest reduction in tumor burden, even when using highly immunogenic OVA expressing MC38 colon carcinoma (FIG. 1A), while deletion in either CD8+ T cells (FIG. 1B) or Tregs (FIG. 1C) had no effect on tumor growth. In addition to T cells, Tim-3 is also expressed on NK cells⁶ however specific deletion on NK cells using NCR1 cre had no effect on tumor growth (FIG. 1D). These observations, albeit surprising, led to the hypothesis that the role of Tim-3 in regulating anti-tumor immunity is predominantly mediated through its action on other immune cells, including myeloid cells (macrophages and monocytes)^{7,8} and dendritic cells, where Tim-3 is constitutively expressed at high levels.⁹⁻¹¹ Recently, the importance of Tim-3 expression on myeloid cells has come to light from observations made in human disease whereby germline mutations in HAVCR2, the gene encoding Tim-3, are associated with spontaneous myeloid cell hyperactivation with elevated circulating levels of IL-1 and TNF-α.¹² Furthermore, the therapeutic efficacy of blocking antibodies to TIM-3 as a monotherapy in myeloid malignancies, especially in AML and MDS,¹³⁻¹⁵ raised the question of whether Tim-3 may play a role in regulating myeloid cell function. These clinical observations together with results showing only modest effects on tumor growth inhibition when Tim-3 was deleted on T cells (FIGS. 1A-C) provided the impetus to look at other cell types in the TME that may express Tim-3.

To fully probe the expression profile of Tim-3 across immune cells, scRNA-Seq was performed on tumor infiltrating CD45+ leukocytes, and CD45+ cells from the draining and non-draining lymph nodes of WT MC38-OVA tumor bearing mice. In lymphoid tissues Havcr2 expression is most prominent in a cluster of cells expressing canonical DC1 markers including Xcr1, Clec9a and Flt3 (FIG. 1E, Extended Data FIG. 1b). However, in tumor CD45+ cells, Havcr2 was expressed in multiple cell types including DCs (clusters 2, 5 & 12), CD8+ T cells (cluster 7), FoxP3+ Tregs (cluster 8), monocytes (cluster 3), and macrophages (cluster 1) (FIG. 1E). These expression patterns were detected at the protein level, with flow cytometric analysis on CD45+ TILs from MC38-OVA (FIG. 1F). In line with the sc-RNAseq data, the inventors found the highest expression of Tim-3 on DC1 (CD11b- XCR1+/CD103+) and Migratory DC (CD11b+CD103+) (FIG. 1G), a subset of DC that shares properties of both DC1 and DC2, and has been proposed in several studies to be important in mediating robust anti-tumor immune responses.^{16,17} Abundant expression of Tim-3 was detected on intratumoral macrophages and monocytes. These data, together with clinical observations made regarding the role of Tim-3 in myeloid cells in human disease,^{12,18} motivated the inventors to systematically investigate the effect of deletion of Tim-3 in monocytes, macrophages, and DCs on tumor burden. LysM cre and CX3CR1cre were used first to achieve a very efficient deletion of Tim3 on macrophages, monocytes, and DC2 populations (Extended Data FIG. 1c, d), but there was no effect on tumor burden observed (FIG. 1H, 1I), excluding the role of Tim-3 on macrophages, monocytes and DC2s in regulating anti-tumor immunity in these models.

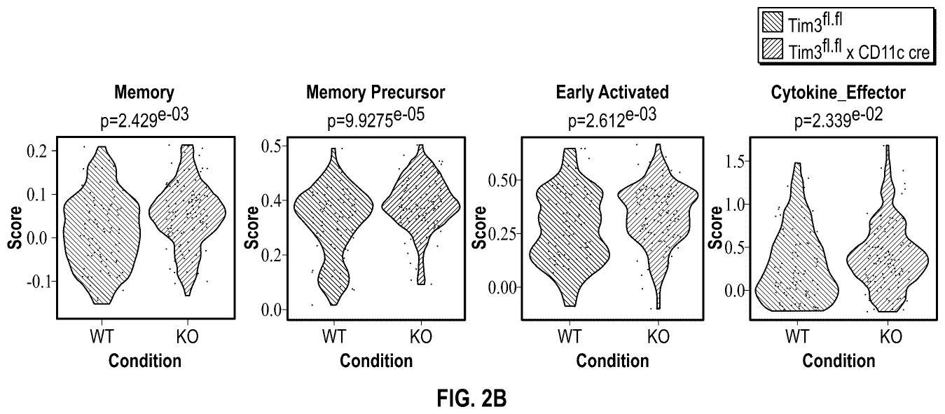

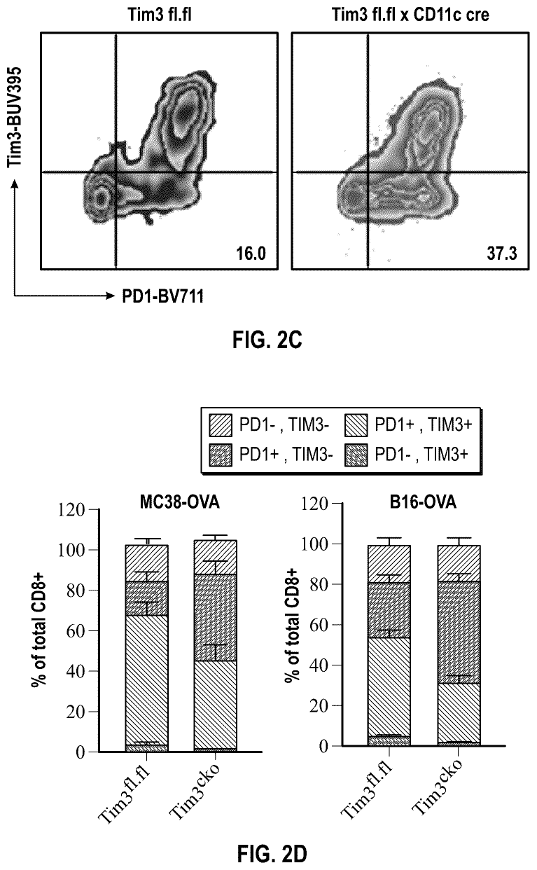

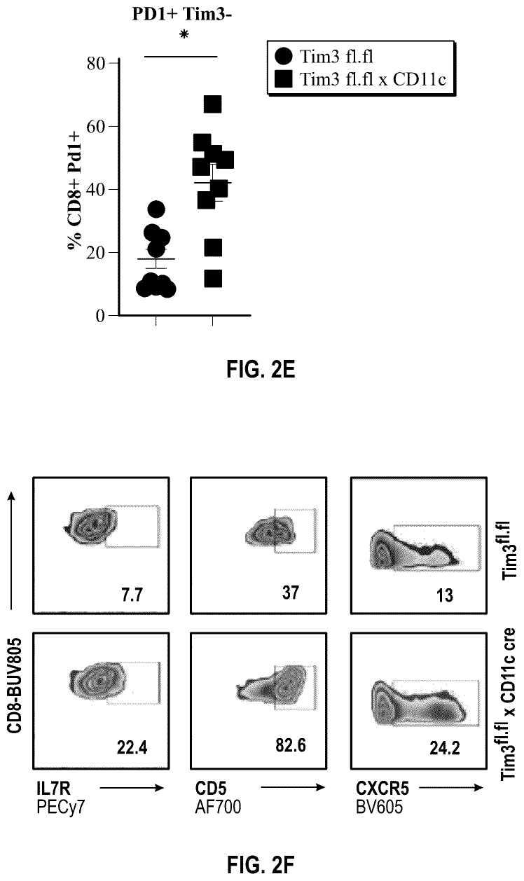

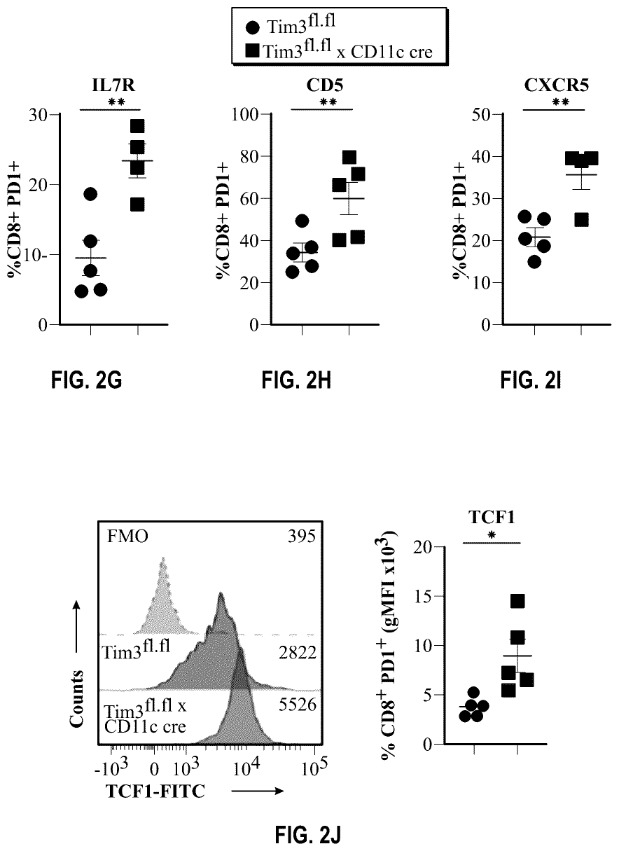

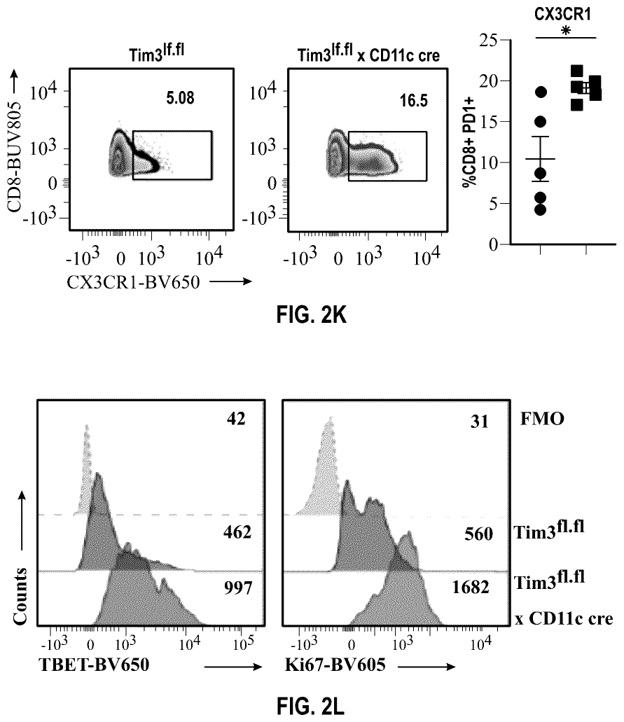

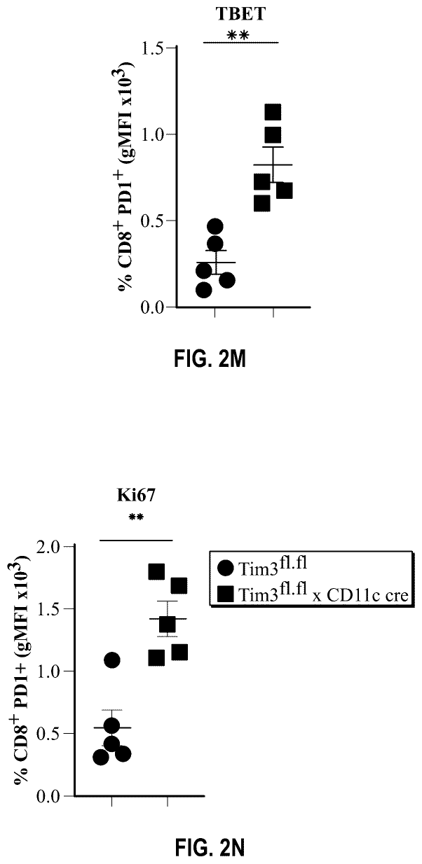

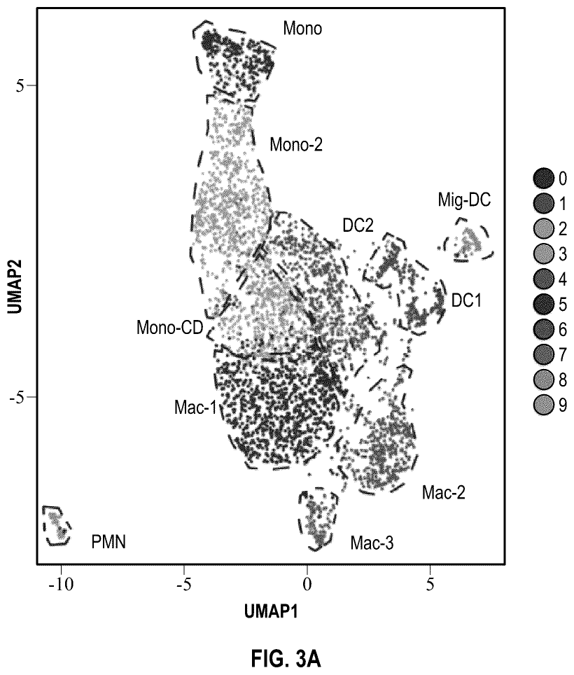

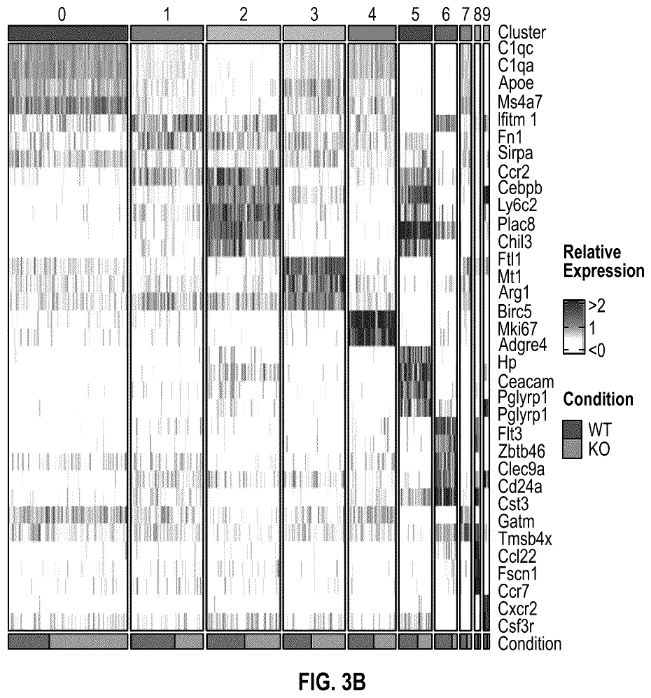

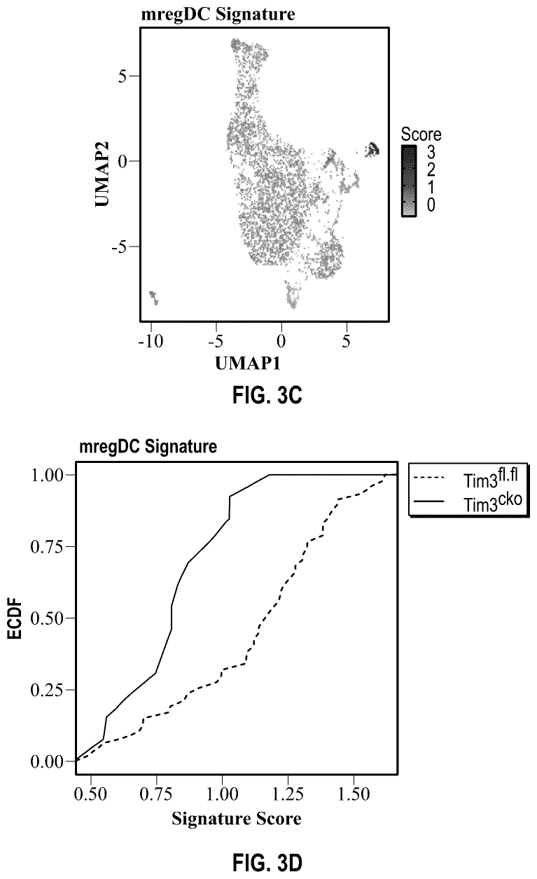

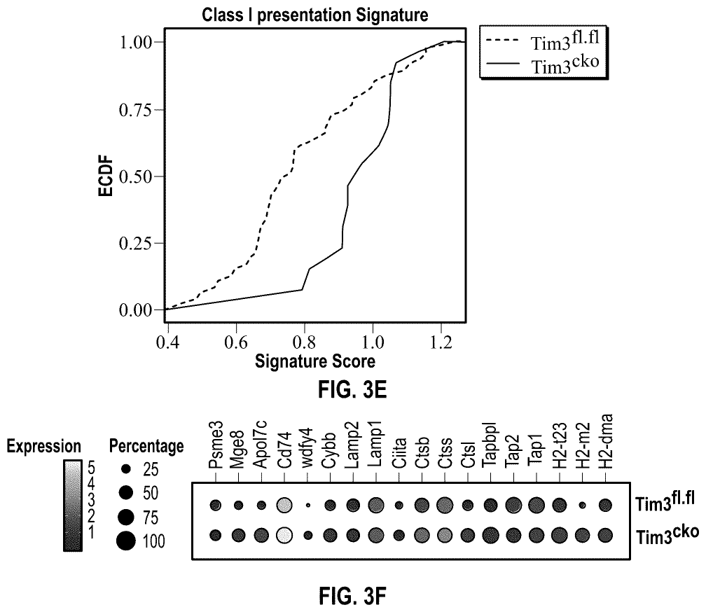

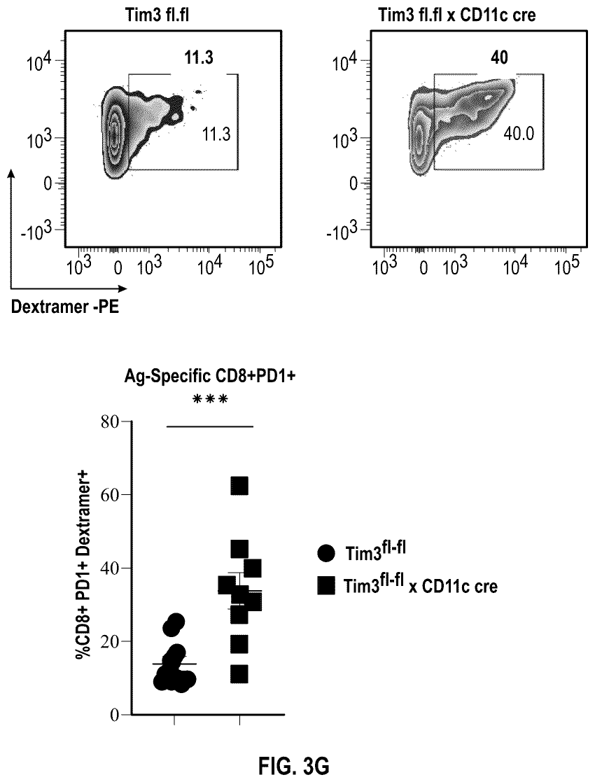

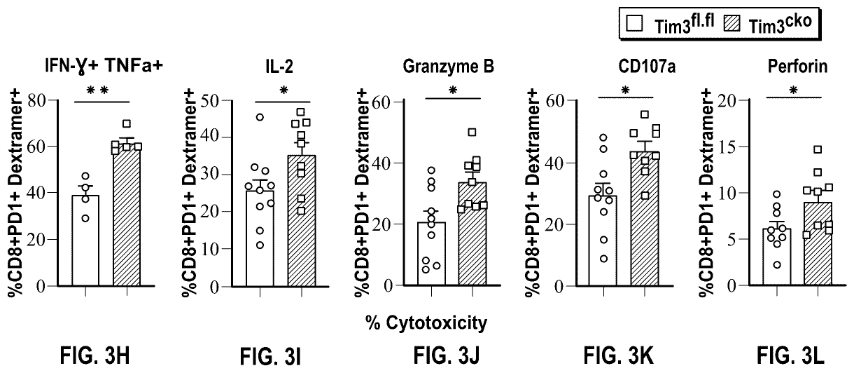

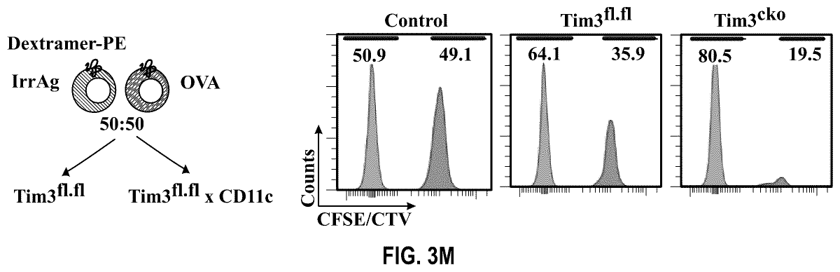

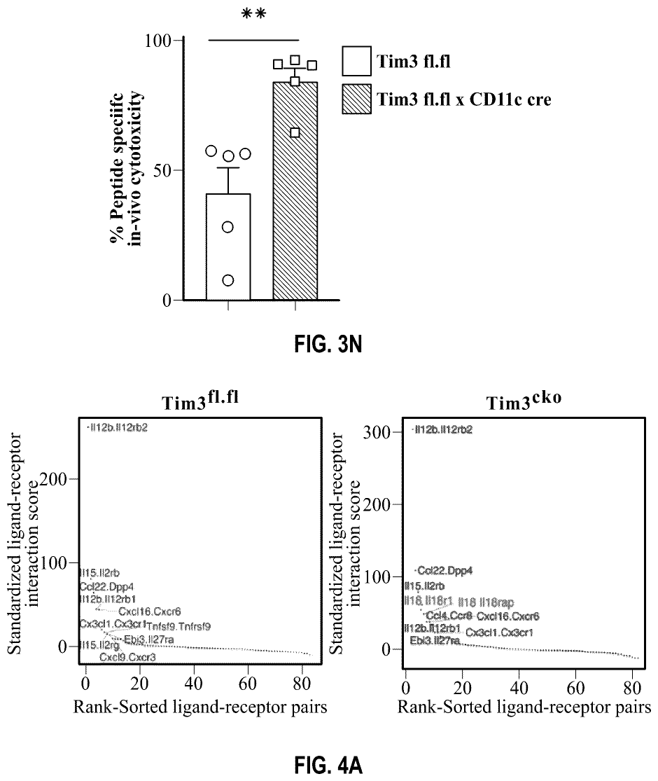

In contrast, when CD11c cre was used to broadly delete Tim-3 in DC populations, significantly reduced tumor growth was observed (FIG. 1J). An additional WT control using Tim3fl.wtxCD11cre ruled out any contribution of CD11c cre itself on tumor reduction (Extended Data FIG. 2a). The inventors further investigated the effect of Tim-3 deletion on DC in a model for non-small lung cancer utilizing orthotopic syngeneic KP1.9 lung adenocarcinoma cells, which have been derived from lung tumors of C57BL/6 KP mice harboring Kras and Trp53 mutations.¹⁹ Using this model with cells derived from primary tumors, the inventors also found that Tim-3cko animals had significantly reduced tumor burden compared to WT controls (FIG. 1K). To confirm that these findings were intrinsic to loss of Tim-3 on DCs, transfer experiments were performed where DCs were generated from bone marrow progenitors (FIG. 1I; Extended Data FIG. 2b) of wild type or Tim3fl.flxCD11c cre mice, loaded them with OVA, and transferred into B16-OVA-tumor bearing WT mice. Importantly, these experiments showed that intrinsic loss of Tim-3 on DC is sufficient to drive anti-tumor immune responses leading to significantly increased frequency of tumor-antigen specific CD8+ T cells (Extended Data FIG. 2c), with increased cytokine production and cytolytic activity (Extended Data FIG. 2d-g). Although significant deletion of Tim-3 on non-myeloid cells using CD11c was not observed (Extended Data FIG. 1e, f), it is possible that CD11c could be expressed on non-DC populations, including CD8+ T cells and NT cells during inflammation. These results were confirmed using a Zbtb46 cre line that specifically targets cDCs, and a significant reduction in tumor burden was shown in the MC38-OVA model (FIG. 1M). Notably, the Zbtb46 cre had significant deletion of Tim3 in DC1 and MigDC but with minimal deletion on other DC populations (Extended Data FIG. 1c,d; 3a). Since MC38-OVA is an immunogenic tumor model, the inventors also tested the less immunogenic tumors MC38 (FIG. 1N, Extended Data FIG. 3b) and B16F10 melanoma that do not also express an exogenous antigen (Extended Data FIG. 3c) and again found that loss of Tim-3 on DCs led to significant inhibition of tumor growth.

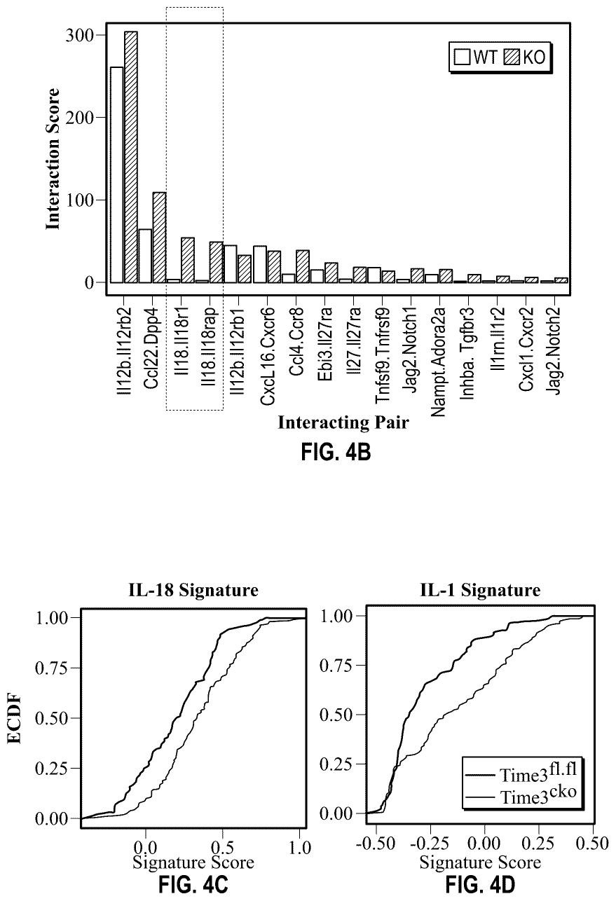

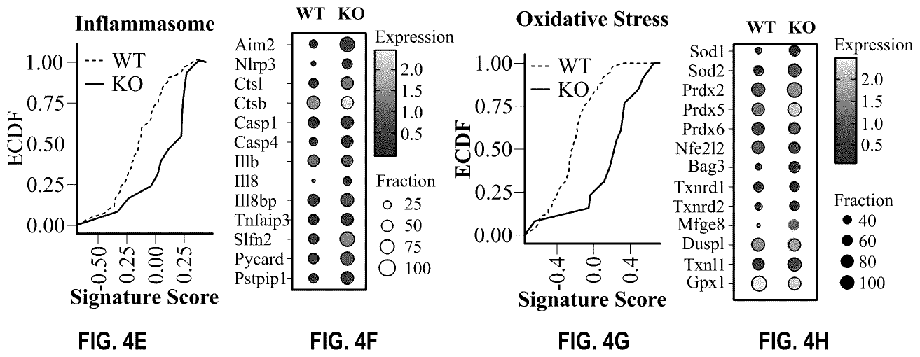

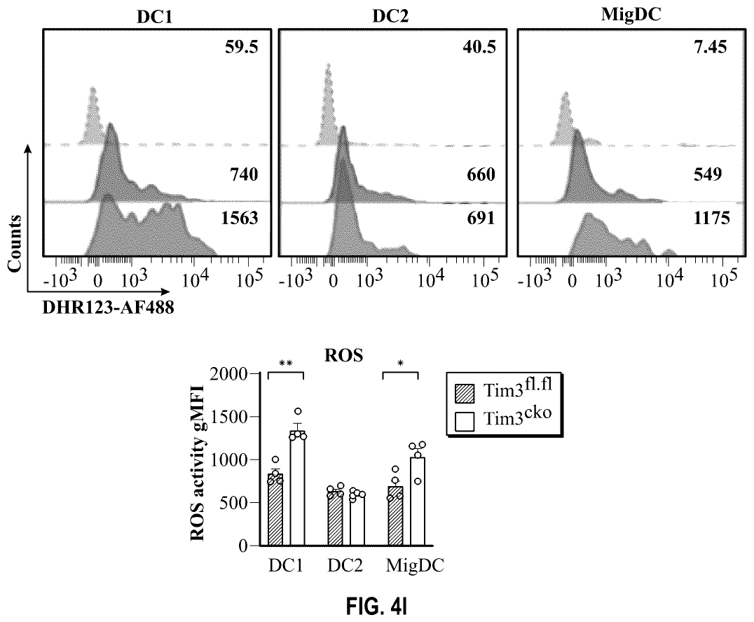

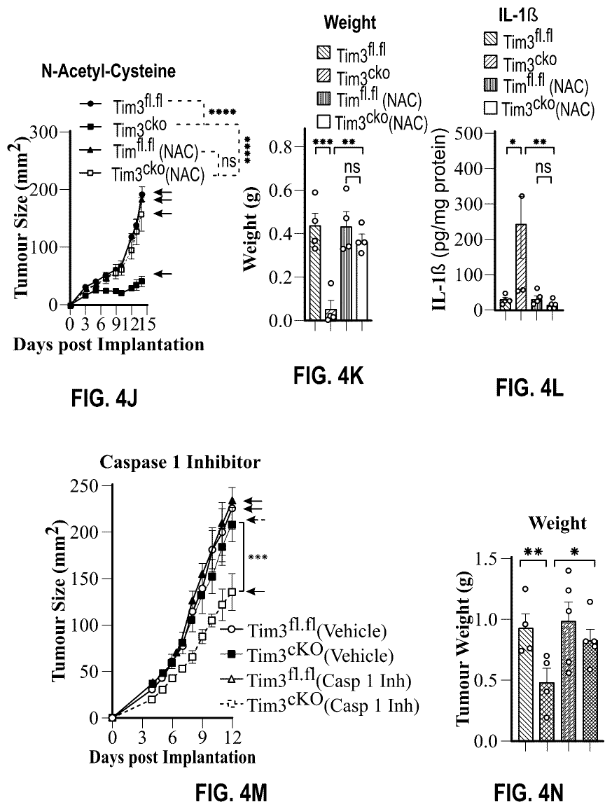

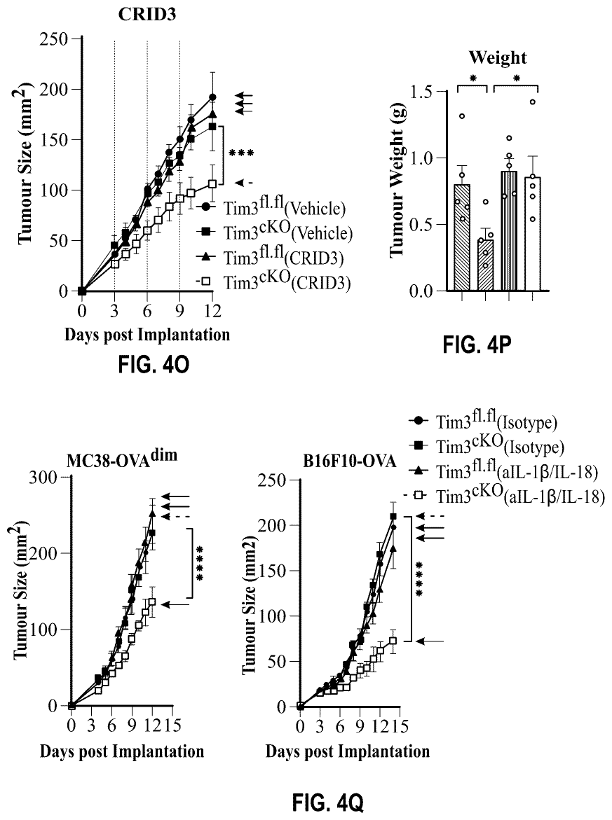

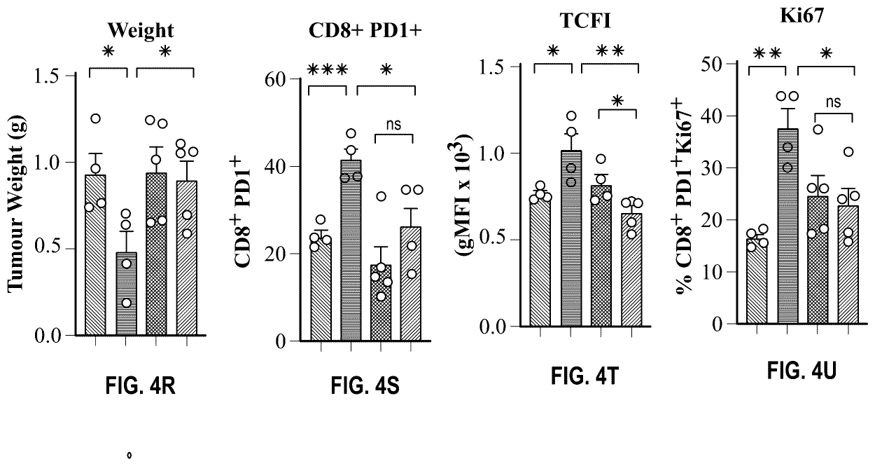

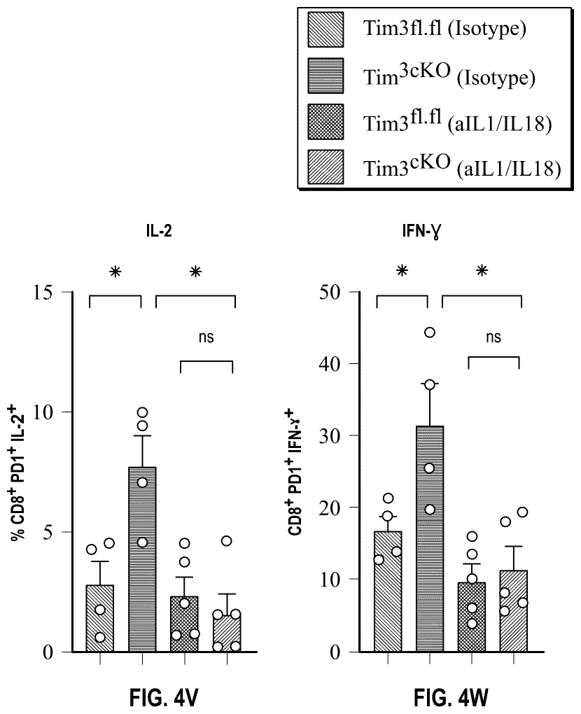

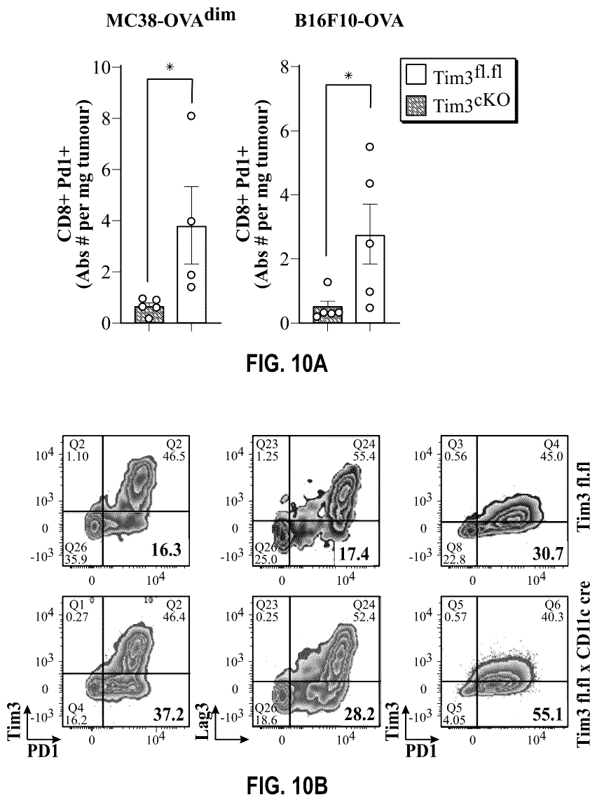

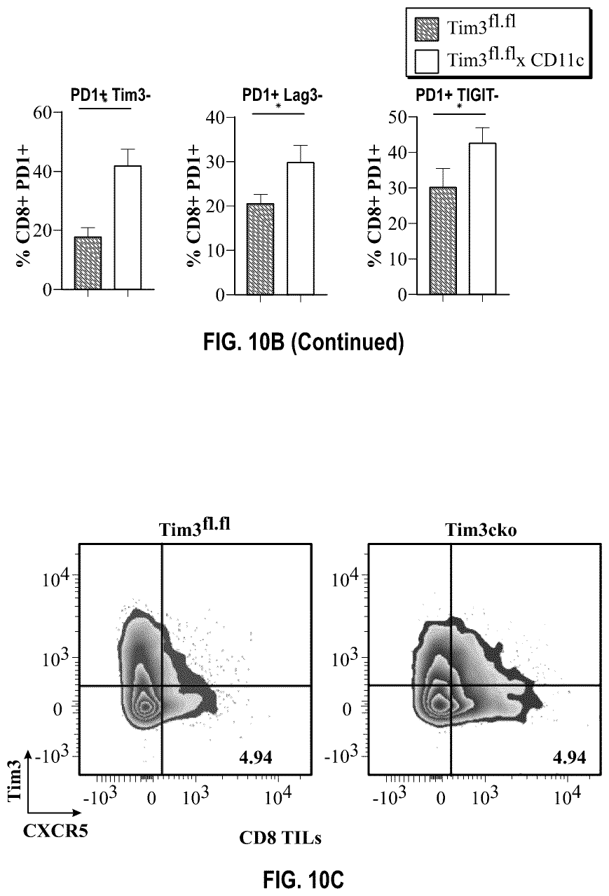

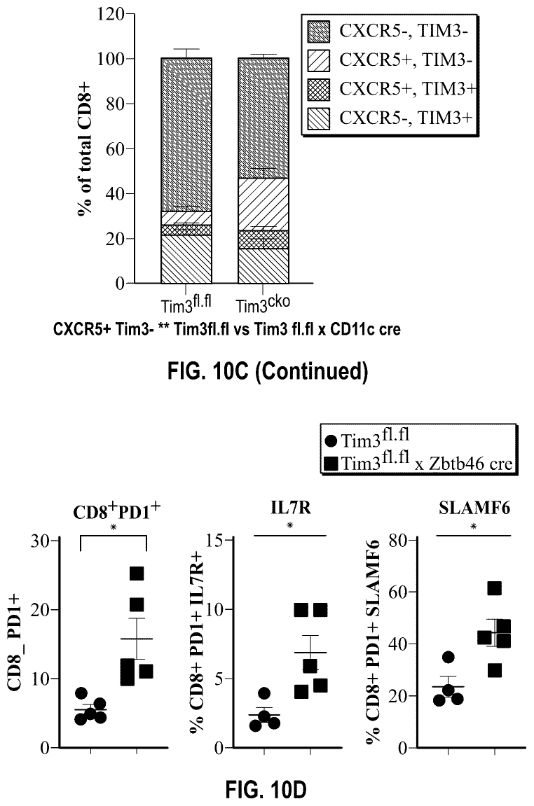

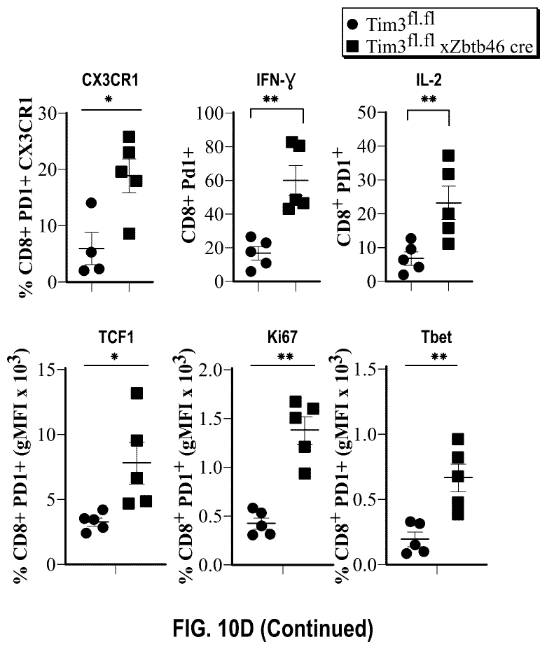

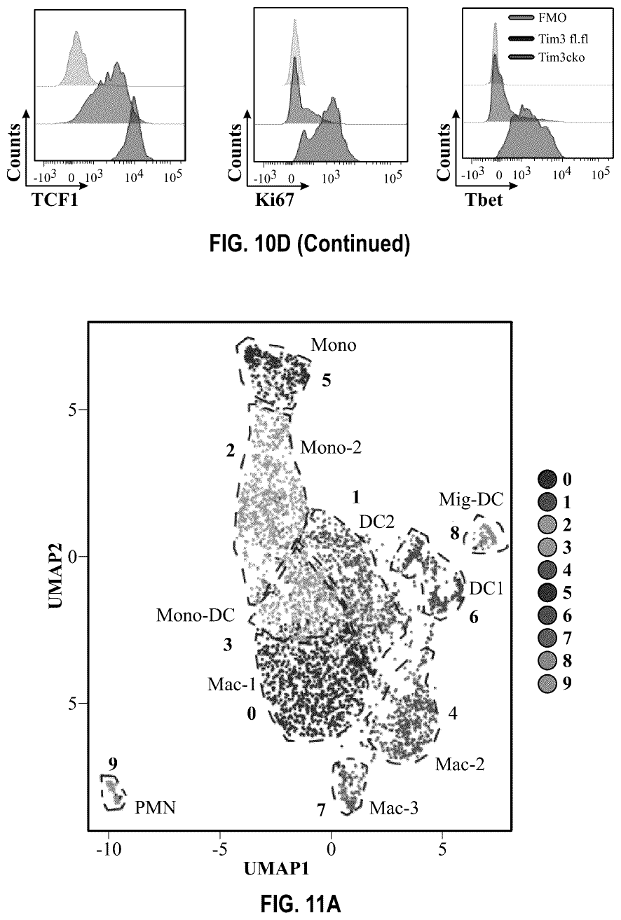

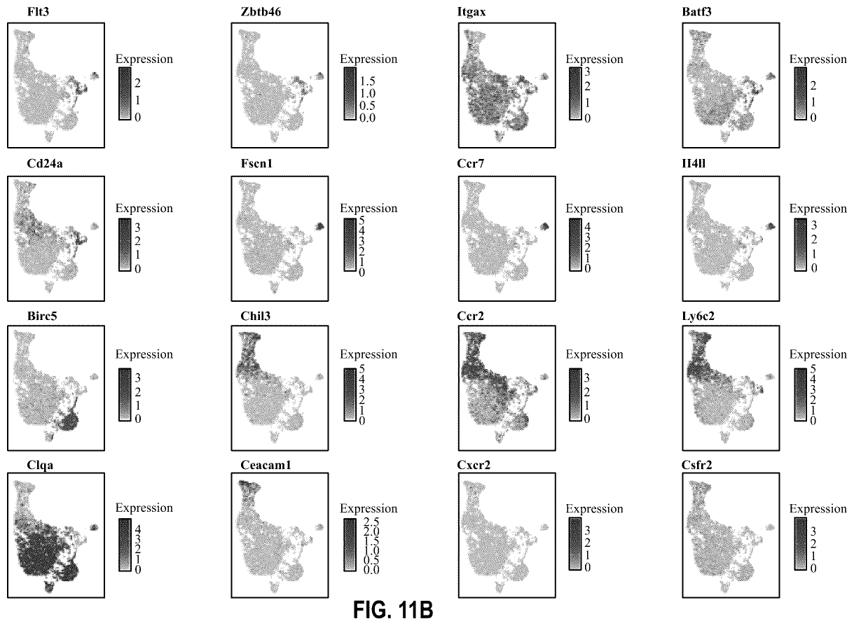

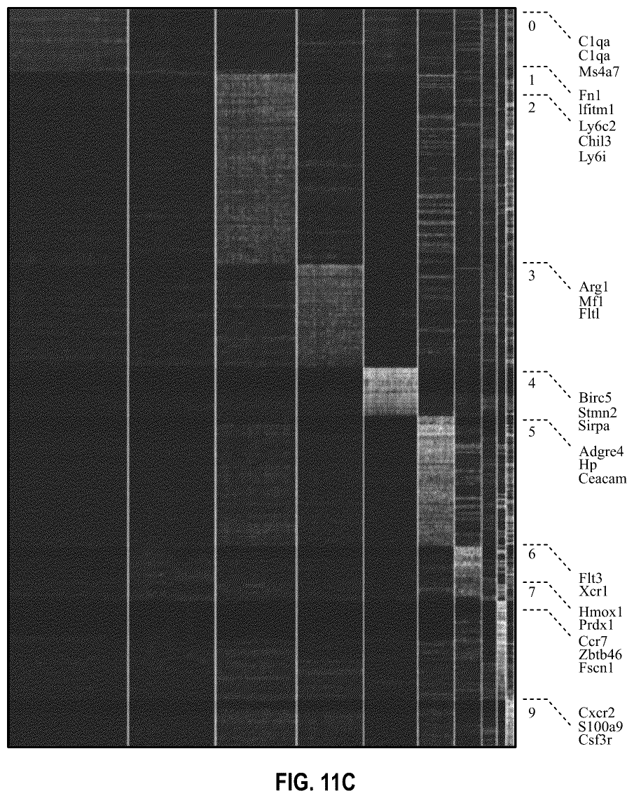

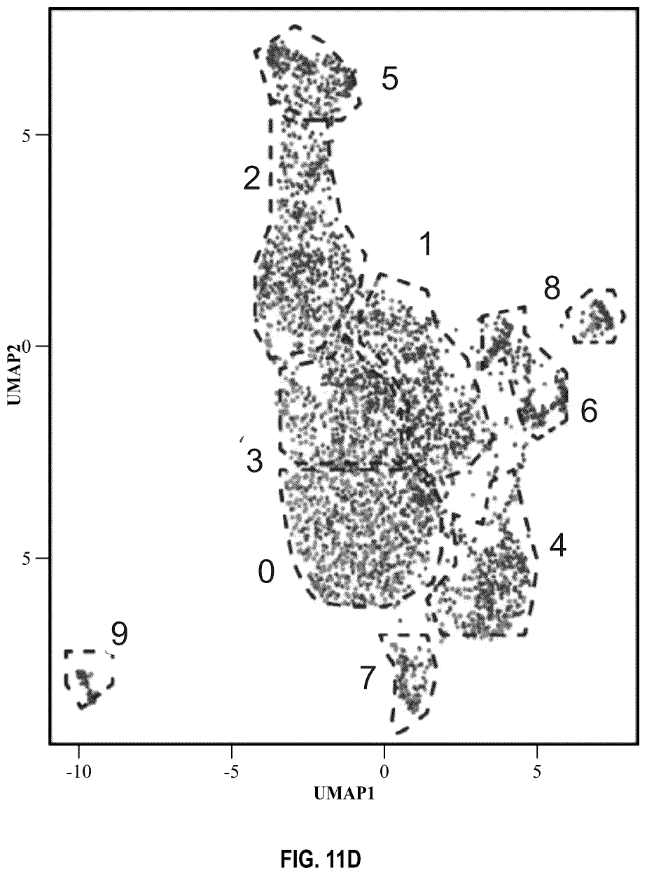

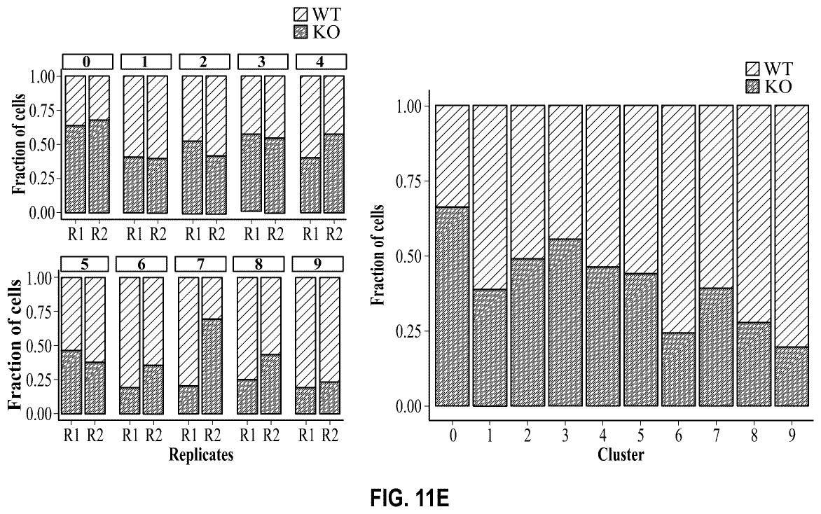

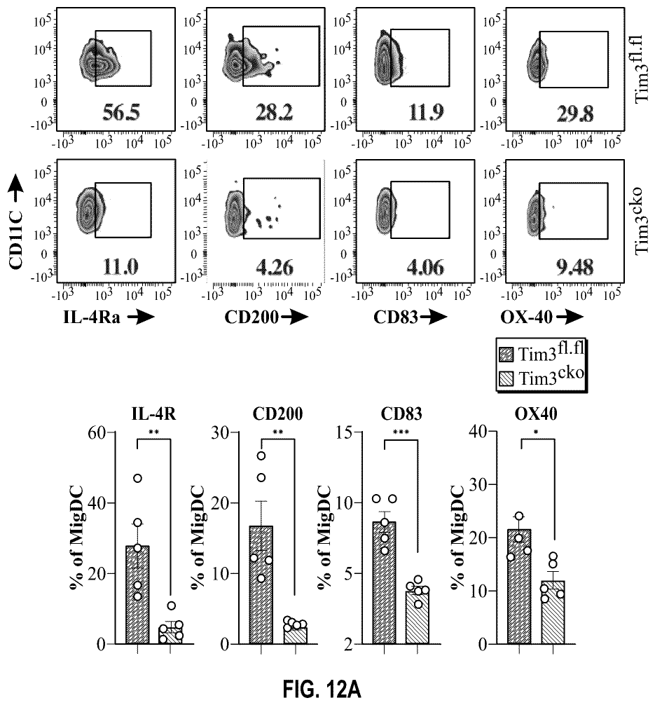

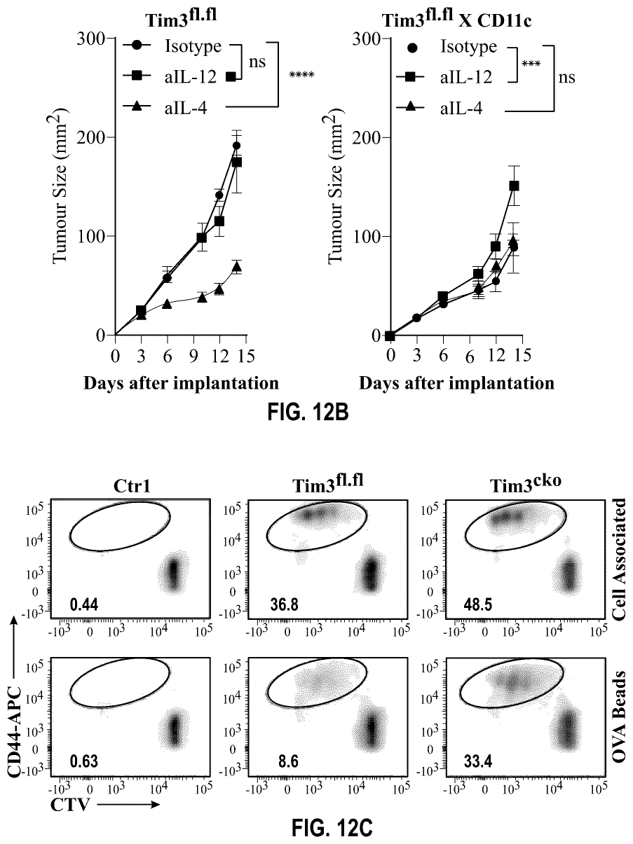

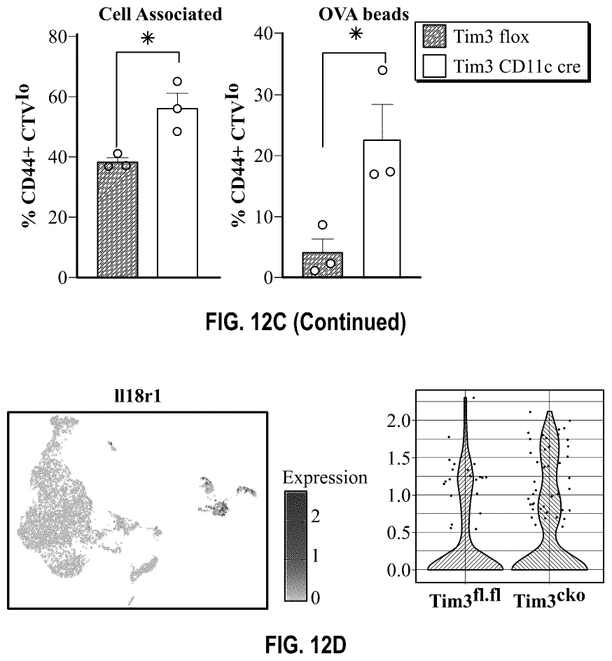

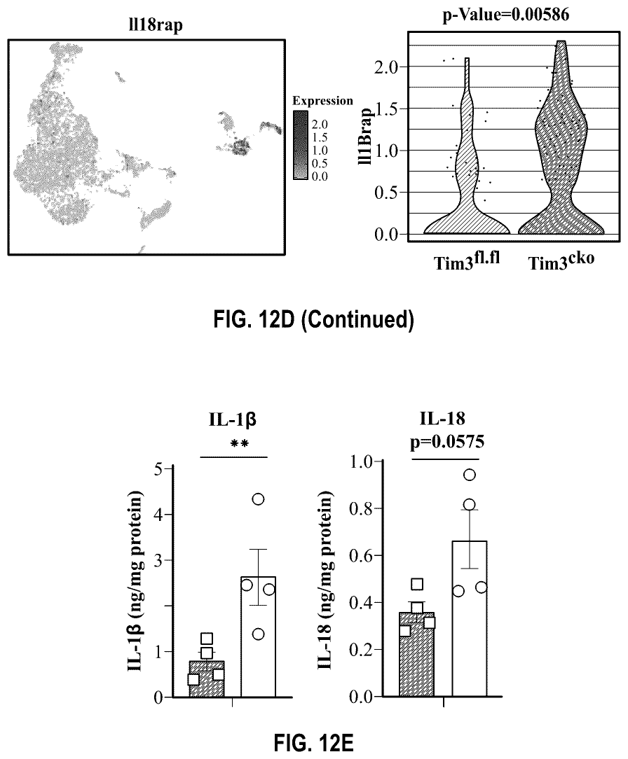

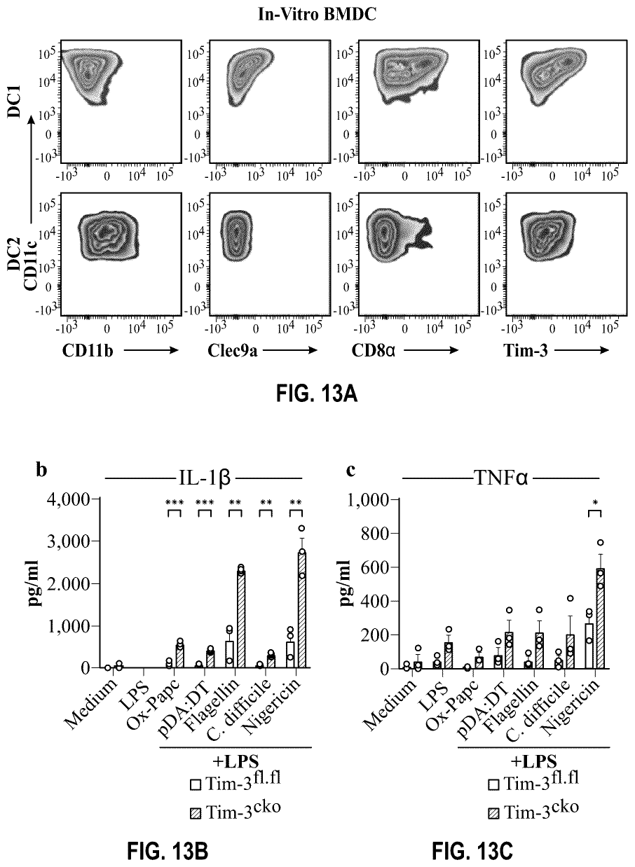

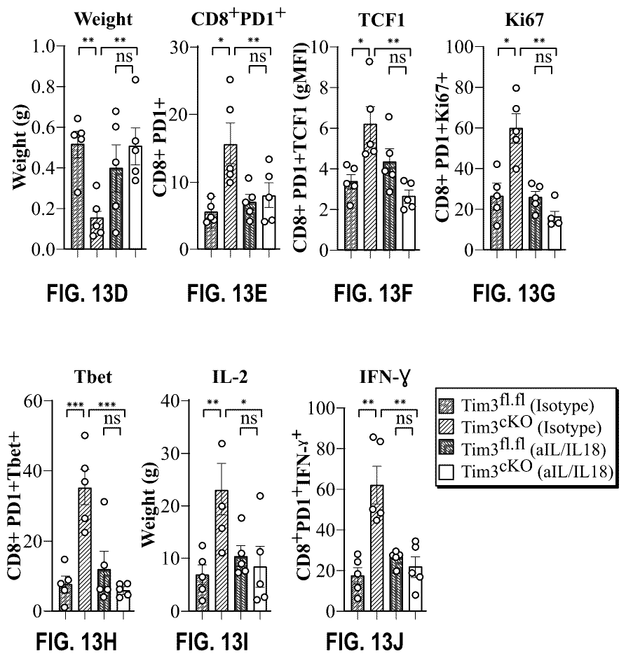

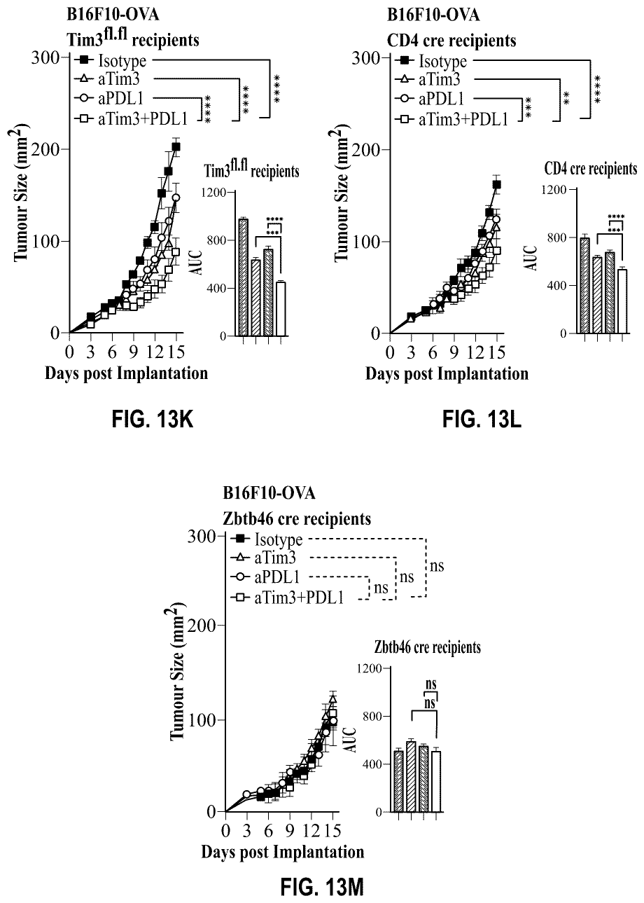

The inventors further compared deletion of Tim-3 on DC and T cells in the same experiment and found that deletion of Tim-3 in DC populations led to superior inhibition of tumor size (FIG. 10) and weight (FIG. 1P) when compared to its deletion in T cells, which was also observed using B16F10 and B16-OVA (Extended data FIG. 3d-f). There were no significant changes in cellular frequencies of DCs, monocytes, macrophages, NT cells, B cells, T conventional, and Tregs infiltrating the tumor between Tim-3fl.fl and Tim-3fl.flxCD11c cre mice (Extended Data FIG. 4a, b-k). However, looking within the T cell compartment, there were significantly elevated numbers of CD8+ T cells infiltrating Tim-3fl.flxCD11c cre tumors (Extended Data FIG. 4l). As the highest Tim-3 expression was observed on DC1 and MigDC (FIG. 1G), the inventors assessed the expression of typical cytokine, chemokine, co-inhibitory, and co-stimulatory molecules on intratumoral DC1 and Mig DC, including CXCL9, IL-12, IL-10, IL-27, CD86 and CD40 (Extended data FIG. 4m) but found no significant changes between Tim-3fl.fl and Tim-3fl.flxCD11c cre, with the exception of CD24 which was consistently higher on Tim-3 deficient DC1 and MigDC.

Together these results indicated that Tim-3 plays a dominant role in regulating DC function to promote anti-tumor immunity, prompting the inventors to comprehensively investigate changes within the TME between Tim-3fl.fl (WT) and Tim-3fl.flxCD11c (Tim-3cko) tumors.

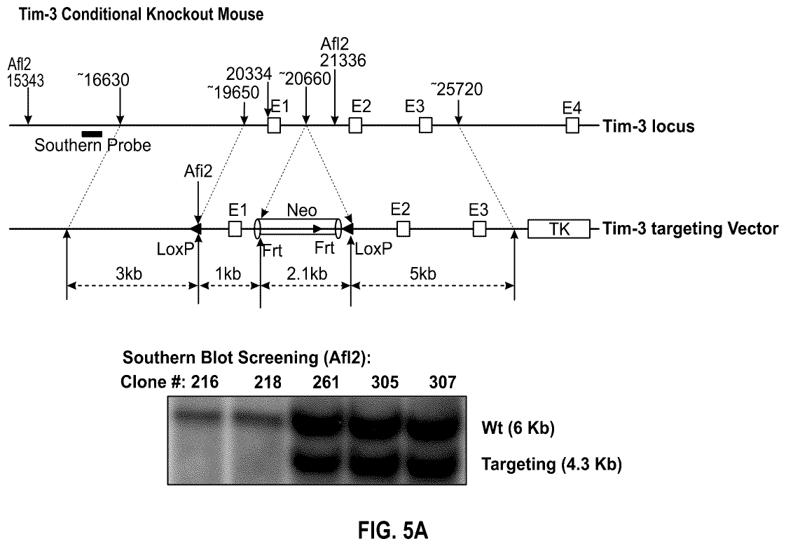

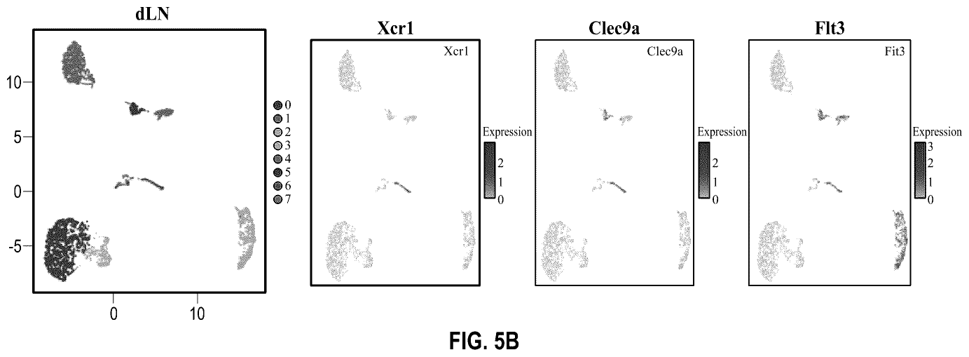

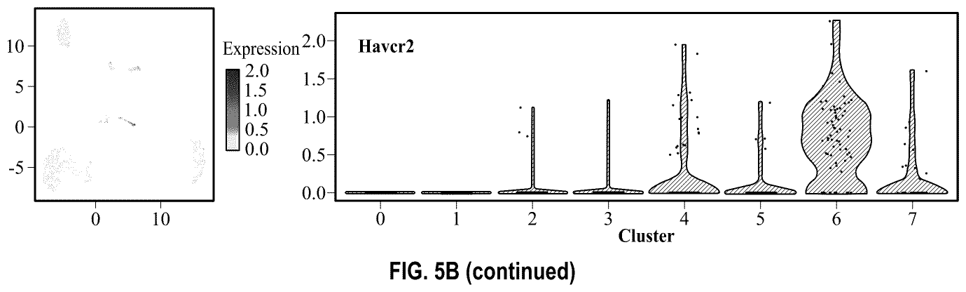

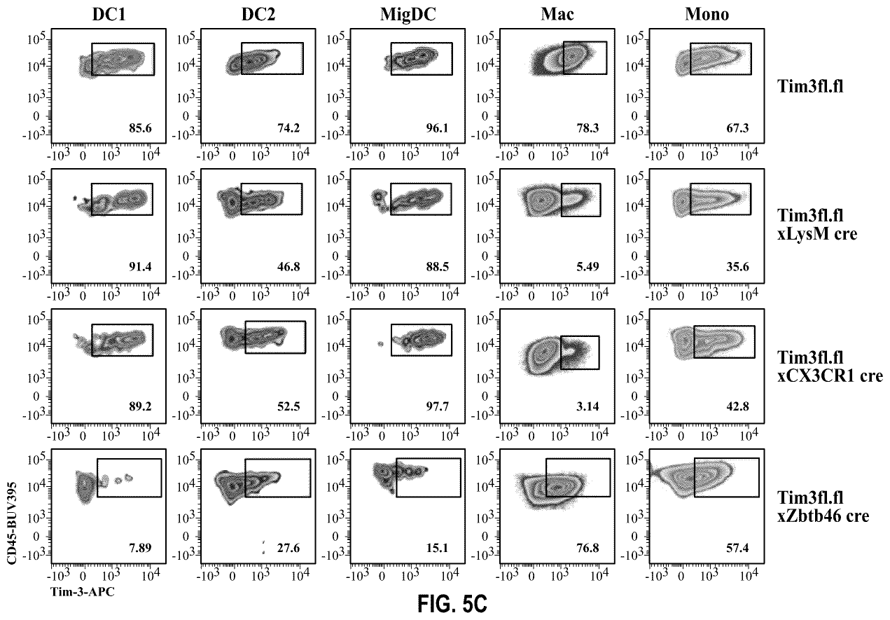

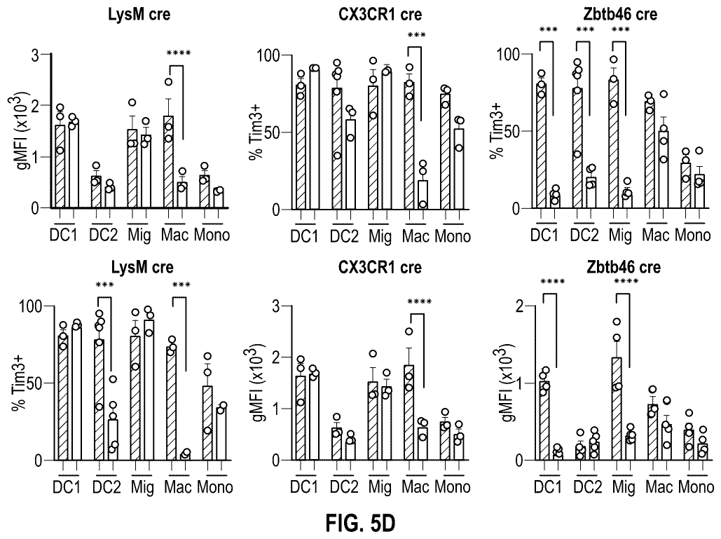

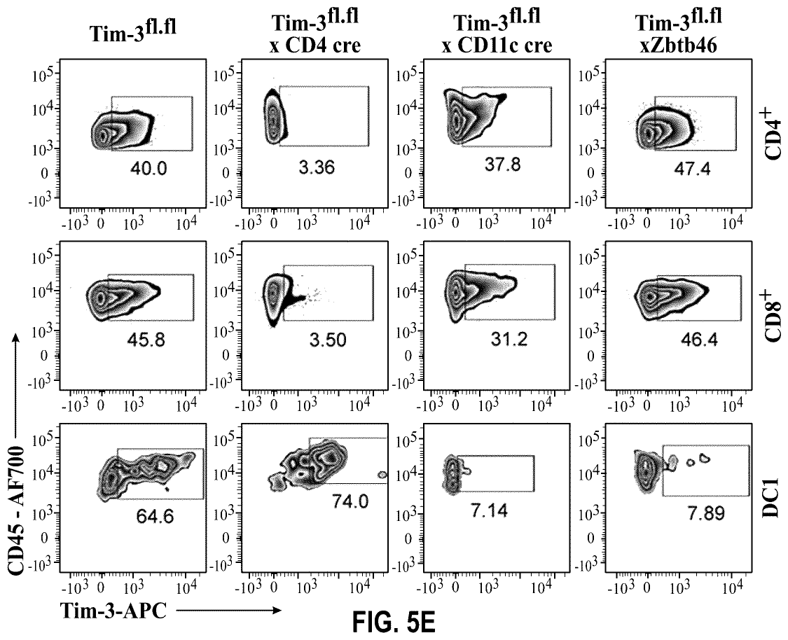

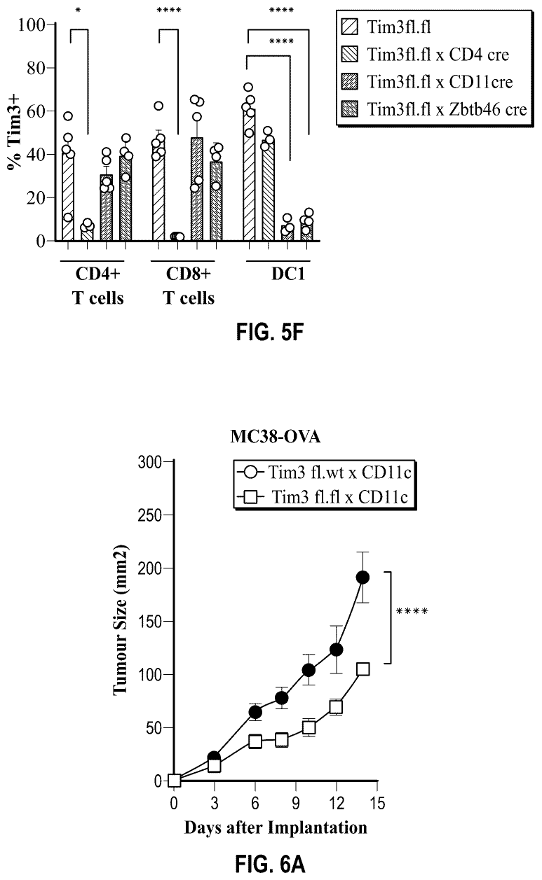

Using scRNA seq the inventors profiled tumor infiltrating cells from MC38-OVA bearing WT and Tim-3 3^{cko}, using cell sorting to enrich for rarer immune populations, including DCs. Based on the expression of known cell-type specific markers, and comparisons with existing single cell datasets,^{20,21} 15 distinct cell subsets were annotated, including four lymphoid clusters, a single NK cell cluster, seven distinct myeloid clusters, and three conventional DC clusters (Extended Data FIG. 5a-c). All clusters had representation from both WT and Tim-3cko (Extended Data FIG. 5d), but at different proportions. In particular clusters 0 which contained monocyte derived macrophages (p-value=4.74*10−5), clusters 10 & 12 which contained activated B cells (p-value=1.16*10−2 and p-value=4.16*10−3 respectively), and cluster 7 which contained CD8+ T cells (p-value=1.3*10⁻⁴), (Extended Data FIG. 5e), had a higher frequency of cells arising from Tim-3^{cko} tumors (FIG. 2A).

The CD8+ T cells in cluster 7 from Tim-3^{cko} tumor bearing mice expressed higher levels of gene signatures found in a spectrum of functional CD8+ T cell states including signatures of early activated (p-value=2.6*10⁻³)²², effector (p-value=2.3*10⁻²)²³, memory (p-value=2.4*10⁻⁰³)²³ and memory precursor (p-value=9.9*10⁻⁰⁵)²⁴ cells (FIG. 2B). Memory precursor cells are present within the effector pool of CD8+ TILs, where they remain primed and maintain a steady homeostatic proliferative capacity. These “stem-like’ CD8+ T cells have been identified in several human cancers^{22,25,26} and have been shown to be essential for providing a proliferative burst that supplies the effector T cell pool required for sustained anti-tumor immunity in response to checkpoint immunotherapy^{24,27,28}. Thus far, the stem-like cell population in the tumor microenvironment has been shown to be regulated at the chromatin level in a T cell intrinsic manner²⁹ and it remains unclear what cues, if any, within the TME, including from APCs, can contribute to the expansion and maintenance of this important precursor pool.

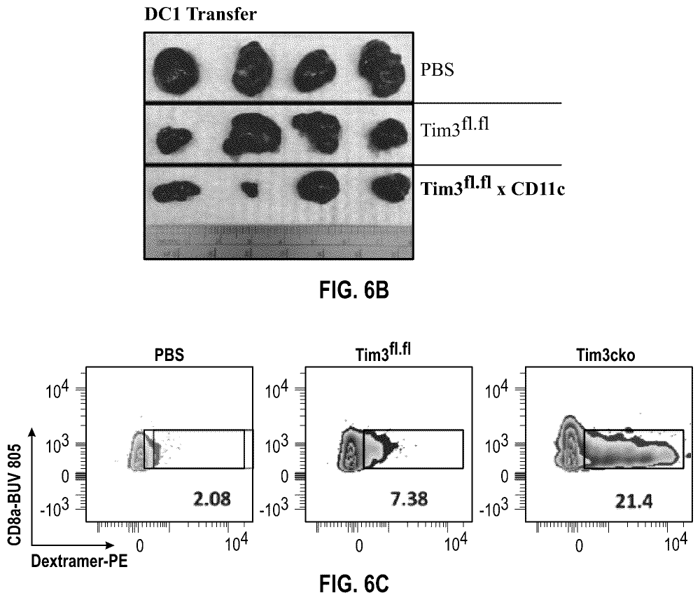

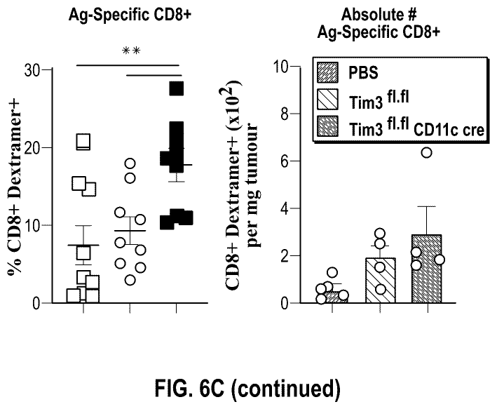

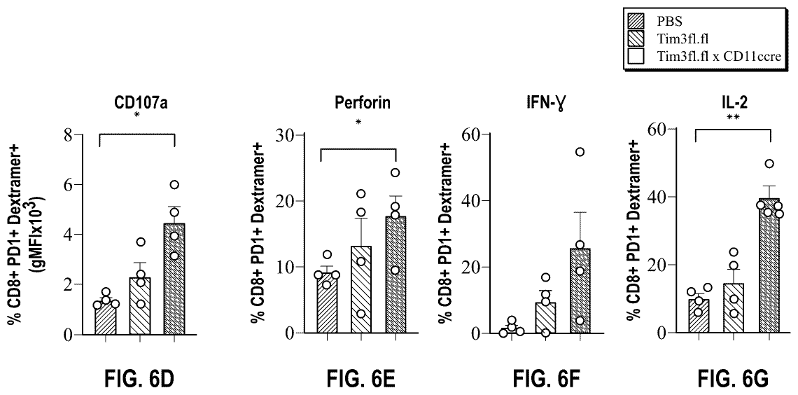

Therefore, it was hypothesized that loss of Tim-3 on DCs could contribute to induction or maintenance of stem-like progenitor CD8+ T cells. Stem-like CD8+ T cells express low levels of PD-1 but lack Tim-3 expression. Supporting this hypothesis, the inventors observed a significant increase in the frequency (FIG. 2C-2E) and absolute number (Extended Data FIG. 6a) of PD-1 single positive (CD8+PD1+Tim3−) TILs in both MC38-OVA and B16-OVA tumors from Tim-3^{cko} mice. This increase in PD1+ cells was also observed when assessing expression of PD1+Lag3− or PD1+TIGIT− (Extended Data FIG. 6b) indicating that expansion of this PD1+ population could not be simply due to potential partial loss of Tim-3 on CD8+ T cells using CD11c cre. More recently PD1+ TILs have been described to contain both stem-like and effector-like CD8+ TILs.²⁴ Therefore, the inventors assessed the expression of several key cell surface markers that can distinguish these two populations.^{24,30-32} Increased expression of IL-7R, CD5, and CXCR5 was found on PD-1+CD8+ T cells infiltrating the tumor in Tim-3^{cko} mice (FIGS. 2F-2I), indicating increased stem-like CD8+ TILs. In line with this, the inventors observed significantly expanded CXCR5+Tim3−CD8+ T cells in Tim3^{cko} tumors (Extended Data FIG. 6c).

A hallmark of memory precursor stem-like cells is expression of the transcription factor TCF1, which has been shown to be important in curtailing terminal differentiation.³⁰ Accordingly, significantly elevated levels of TCF-1 were found in Tim-3^{cko} mice (FIG. 2J). Increase in the CD8+PD1+ population as well as expression of stem-like CD8+ T cell expressing IL-7R, SLAMF6 and TCF1 was additionally confirmed using Tim-3fl.flxZbtb46 cre (Extended data FIG. 6d). In addition, the inventors found elevated expression of CX3CR1 (FIG. 2K), indicating an increase in cells entering the effector lineage. In line with this, increased expression of Ki-67 and T-bet (FIG. 2L-2N) was observed. All were confirmed using Tim-3fl.flxZbtb46 cre (Extended data FIG. 6d). Taken together, these results indicate that loss of Tim-3 expression on DCs promotes the maintenance of stem-like CD8+ T cells and effector T cells which together promote protective immune responses in Tim-3^{cko} tumors.

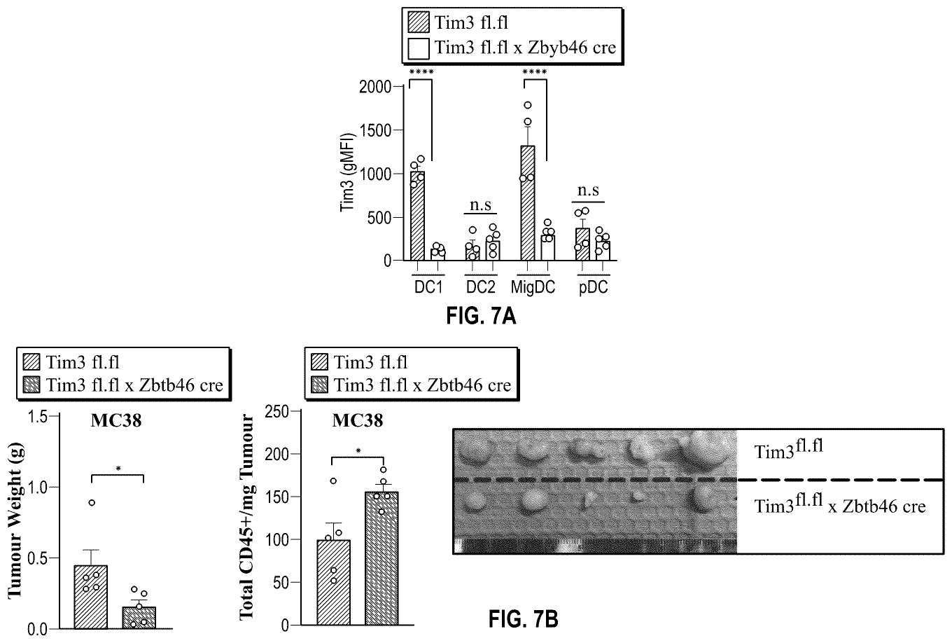

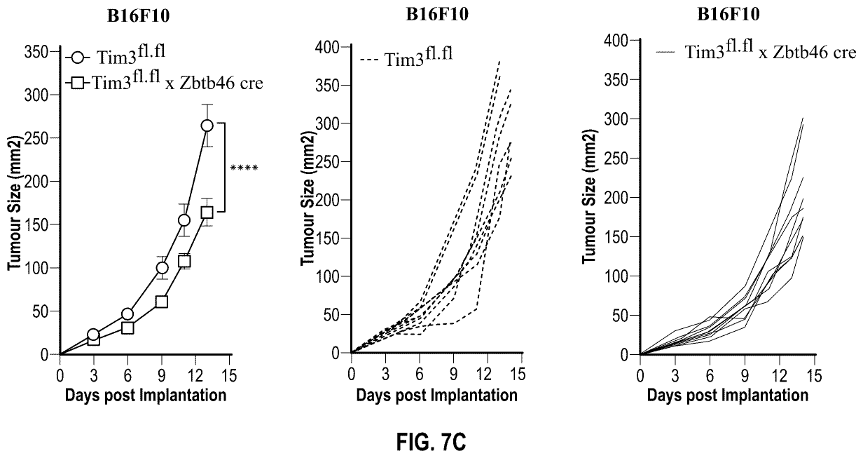

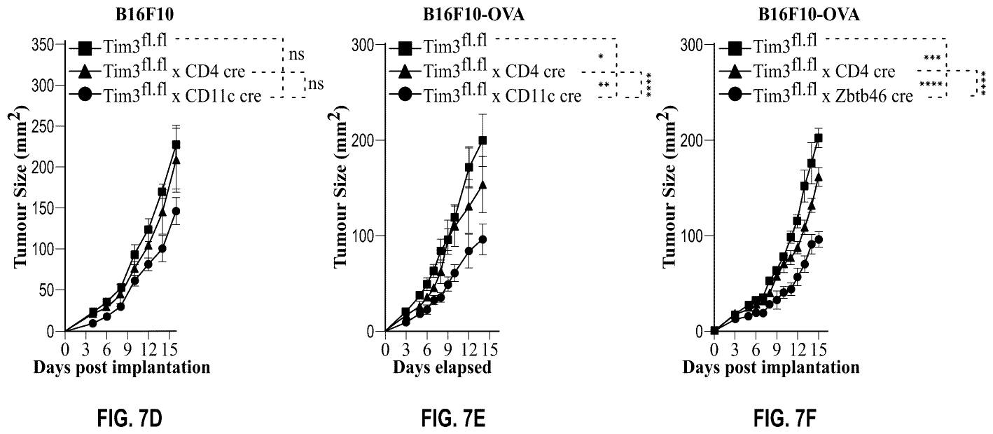

Given the observed transcriptional and functional changes in the CD8+ T cell compartment of Tim-3^{cko} tumors, it was sought to understand what changes within DCs were driving this enhanced effector function in CD8+ TILs. Sub-clustering only the myeloid cells from the single cell profiling dataset, the inventors identified 10 transcriptionally distinct subsets (FIG. 3A; Extended Data FIG. 7a-c). All clusters had representation from both WT and Tim-3^{cko} (Extended Data FIG. 7d-e), but with different proportions. In particular, cluster 0 which comprised monocyte-derived macrophages was highly enriched with cells from Tim3^{cko} tumors. Cells within this cluster had high expression of CCR5, which is consistent with the observation of increased levels of Rantes in supernatants from Tim-3^{cko} tumors (data not shown). Conversely, there was a lower proportion of Tim3^{cko} cells in clusters 9 (neutrophils), cluster 6 (DC1, expressing canonical markers Xcr1, Clec9a and Flt3), and cluster 8 (Migratory DC; Ccr7, Ccl22, Fsen1, Il4il) (FIG. 3B, Extended Data FIG. 7b, c).

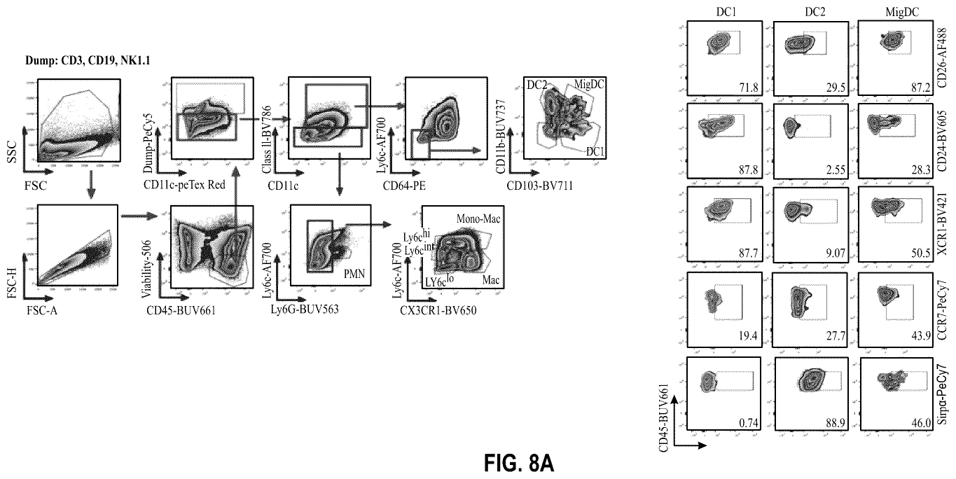

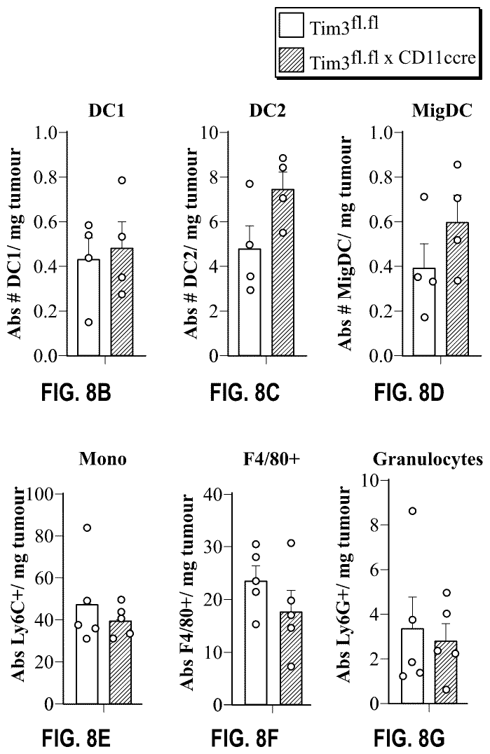

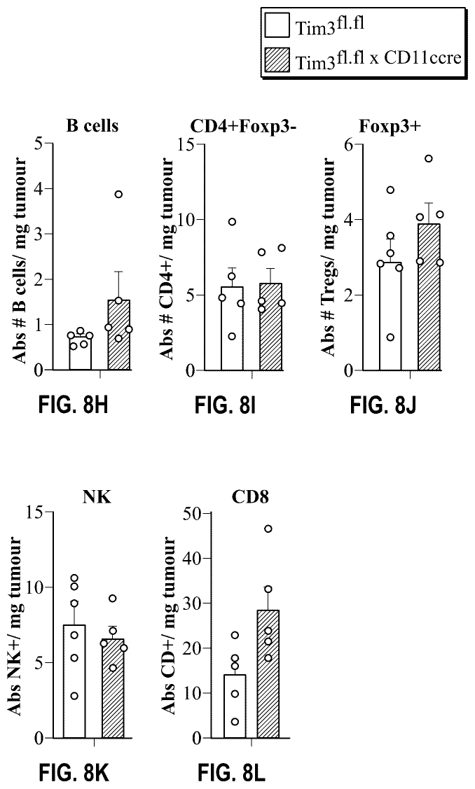

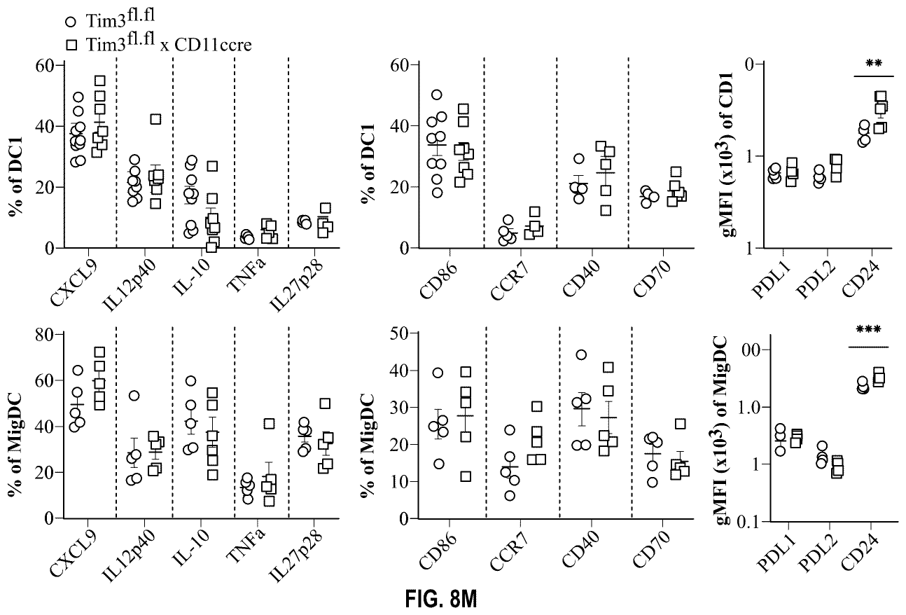

Very recently this migratory population was described to acquire a regulatory transcriptional program in the TME, restraining the immunogenic potential of these cells.¹⁷ Scoring this signature on our scRNA-seq of myeloid cells indeed highlighted cluster 8, migratory DCs (FIG. 3C). The inventors assessed the enrichment score for the regulatory DC signature (mregDC) in cluster 8, and its score was lower in TIM-3^{cko} migDC (FIG. 3D; p-value=9.4*10⁻⁰⁴). In line with the transcriptional analysis, the inventors found that migDC with a deficiency in TIM-3 expressed significantly lower levels of a number of described mreg markers,¹⁷ including IL-4R, CD200, CD83, and OX-40 (extended data FIG. 8A). While induction of this mregDC program has recently been attributed to tumor cell uptake in cancer, it is noteworthy that a similar program has been identified in DCs from other contexts. Previous studies have identified IRF4 as a key transcription factor promoting a Th2 driving regulatory DC program during parasitic infection,³³ and using Perturb-seq the inventors previously identified key positive regulators like RUNX1, IRF4, and PU.1.³⁴ Recently it has been shown that migratory DCs express IL-4Rα and that IL-4 is a key cytokine driving the mreg program, blunting DCI responses. The inventors investigated the effect of IL-4 neutralizing antibodies using anti-IL-12 as a control in TIM-3^{cko} mice versus WT tumor bearing mice (extended data FIG. 8B). It was found that anti-IL-12 partially reversed the anti-tumor protection observed in TIM-3^{cko} with no impact on tumor growth in WT animals. Interestingly, treatment with anti-IL-4 lead to a profound delay in tumor growth in the wild-type cohorts but had no effect on tumor burden in TIM-3^{cko}. This indicates that deficiency of TIM-3 can curtail acquisition of this regulatory program and loss of TIM-3 can enable migratory DC cells to attain a full immunogenic program.

In the context of tumors, a critical function of DCs, most notably DC1 and MigDC^{16,35,36}, is to educate the adaptive immune response, promoting antigen specific CD8+ T cells for durable anti-tumor immunity. The inventors therefore scored the single cells from all myeloid clusters of WT and Tim-3^{cko} tumors for a Class I presentation signature and found significant enrichment of the signature in cluster 8-MigDC, (FIG. 3E; p-value=1.01*10⁻⁰³), where several genes related to class I antigen processing and presentation, including Wdfy4, Cybb, Tapbpl, and H2m2 were upregulated in the cKO cells in the cluster compared to the WT cells (FIG. 3F), the opposite pattern than the mregDC signature. To investigate the role of Tim-3 in cross presentation, the inventors performed in vitro cross-presentation assays using particulate antigen to better mimic uptake of antigen in the tumor microenvironment. Cell trace violet (CTV) labelled OT-I CD8+ T cells, cultured with either dead cell associated OVA or bead coupled OVA, had a higher rate of proliferation and activation (CD44+CTVlo) when cultured with Tim-3 deficient DCs compared to WT DC1 (Extended Data FIG. 8c). In line with this, the inventors found increased frequency of tetramer-reactive antigen-specific CD8+ T cells in Tim-3^{cko} tumors (FIG. 3G), which were particularly enriched in the CD8+PD1+ single positive fraction as described in FIG. 2. Further analysis showed that these cells produced increased levels of effector cytokines IFN-γ, TNF-α (FIG. 3H), and IL-2 (FIG. 3I) with greater capacity for cytotoxicity assessed by increased expression of Granzyme B (FIG. 3J), CD107a (Lamp1) (FIG. 3K) and Perforin (FIG. 3l), which together resulted in increased lytic activity of CD8+ T cells derived from Tim-3^{cko}, in vivo (FIGS. 3M,3N).

Given the observed transcriptional and functional changes in the DC and CD8+ T cell compartment of Tim-3^{cko} tumors, the study sought to understand what changes within DCs were driving this enhanced effector function in CD8+ TILs, and what cellular interactions might be responsible for the effect.

To identify the mediators, potential cell-cell interactions between CD8+ T cells (expanded and activated in Tim-3cko) and MigDC (with enhanced antigen-presentation program) were investigated in Tim3cko derived tumors, leveraging a method the inventors developed³⁷, as part of the Waddington OT package. Briefly, the inventors quantified the potential ligand-receptor interactions by an interaction score defined as the product of the fraction of cells in the cell cluster expressing ligand and the fraction of cells in the cell cluster expressing the cognate receptor. Each ligand-receptor interaction score was standardized by computing the distance between the interaction score and the mean interaction score in units of standard deviations from the permuted data³⁷.

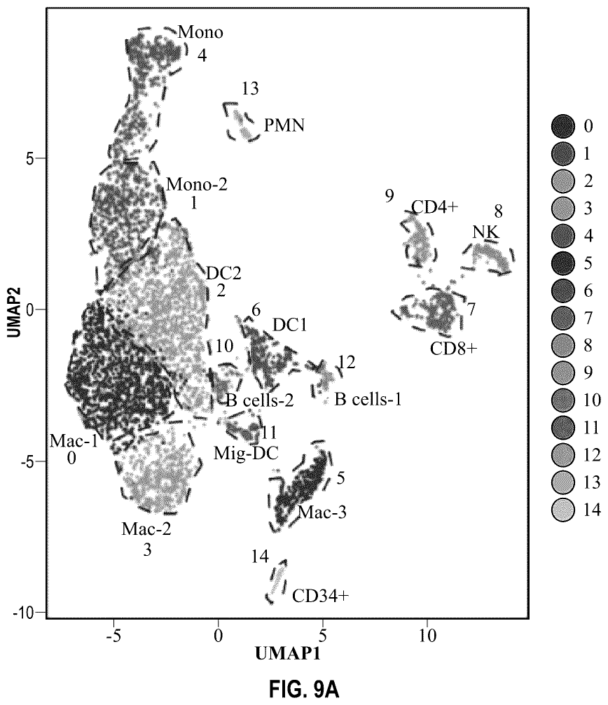

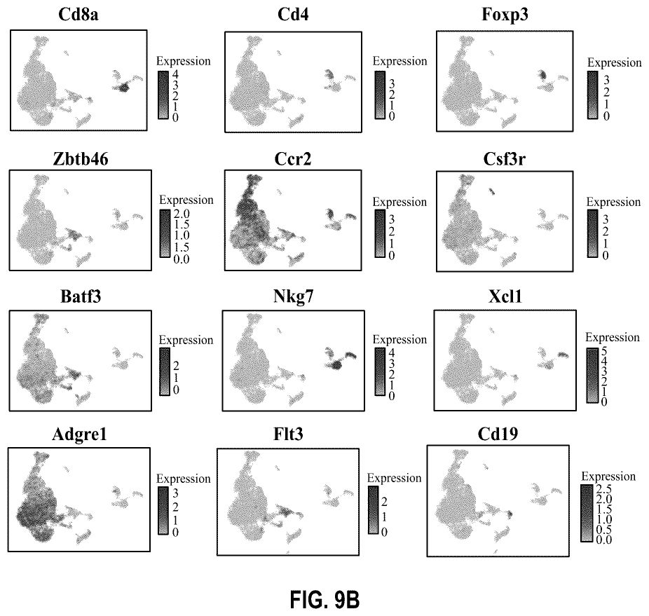

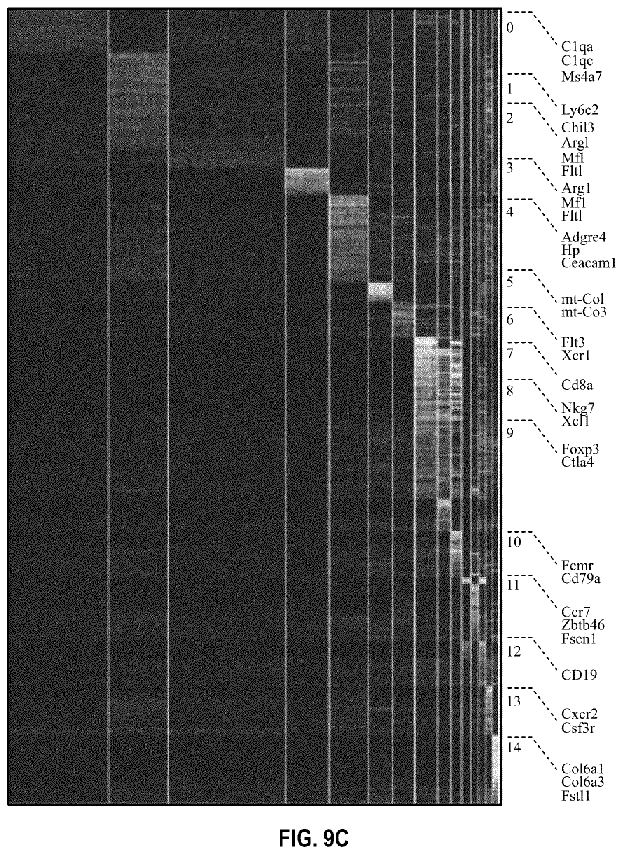

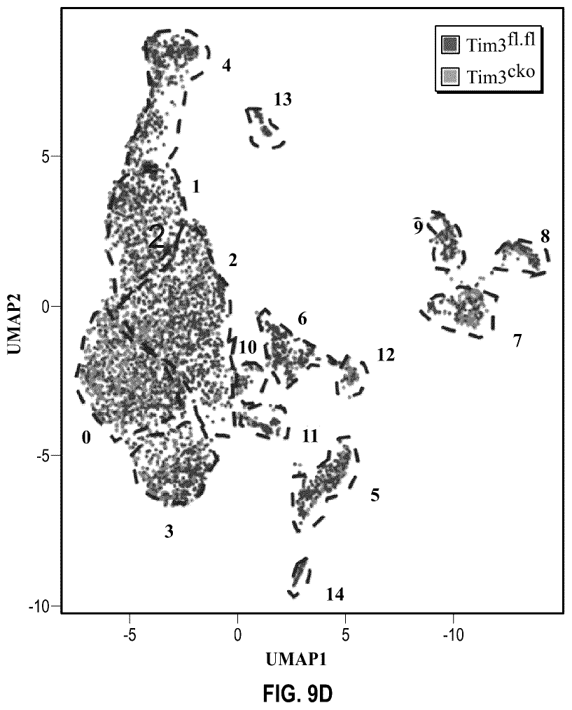

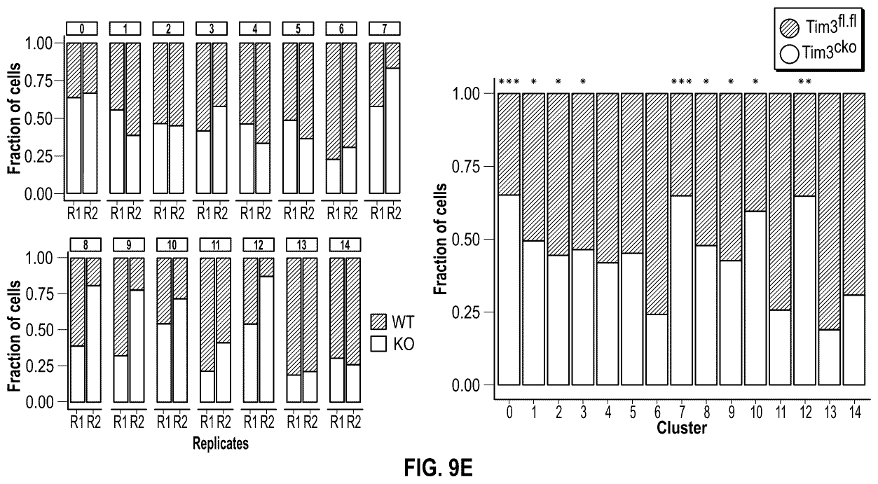

While multiple receptor-ligand pairs scored similarly in cells from WT and cKO tumors, (e.g. Il-12b: Il12rb2), several putative ligand-receptor pairs between CD8+ T cells and DCs had a higher interaction score in Tim-3^{cko} tumors vs. WT, most notably Il-18:Il-18r1 and Il-18:Il-18rap were interactions that scored only in the Tim-3^{cko} tumors (FIGS. 4A, 4B). In line with this finding, Il18rap expression was significantly higher in CD8+ T cells from Tim3^{cko} tumors (Extended Data FIG. 8d). The IlIr1 transcript was not readily detectable in the dataset, but considering the close relationship between IL-1 and IL-18, the inventors scored CD8+ T cells from WT and KO tumors for enrichment of both IL-1 and IL-18 gene signatures and found that both were enriched in CD8+ T cells of the Tim3^{cko} derived tumors vs WT (FIGS. 4C, 4D; p-value=7.02*10⁻⁰⁴ and 2.8*10⁻⁰⁴ respectively). Cytokine analysis of WT and Tim-3^{cko} tumor homogenates also showed higher levels of several inflammatory cytokines, including IL-1β and IL-18 (Extended Data FIG. 8e). IL-1β and IL-18 are both tightly regulated cytokines that are synthesized as precursors which, in permissive conditions, are cleaved and released as mature IL-1β & IL-18 in a process mediated by inflammasomes³⁸, multiprotein complexes that, upon sensing appropriate ligands, can oligomerize and activate caspase family members which then mediate the catalytic conversion of pro-IL-1B or pro-IL-18 to mature IL-1β and IL-18³⁹. Investigating differentially expressed genes between WT and KO the inventors found significant enrichment for an inflammasome gene signature in Tim-3 deficient MigDC (FIG. 4E, p=1.5*10−03) including key adaptor molecules, enzymes and cytokines involved upstream (Aim2, Nlrp3) and downstream (Casp1, Casp4, IL18) of inflammasome activation (FIG. 4F). To investigate the effect of loss of Tim-3 on inflammasome activation in DC, the inventors generated BMDC1 (Extended FIG. 9a) and stimulated with agonists of NLRP3 and AIM2. It was found that activation of NLRP3 with ox-PAPC, or AIM2 with pdA:dT, lead to significantly higher levels of IL-1β in supernatants from Tim-3 deficient DC1 (Extended FIG. 9b). Importantly TNF-α levels were comparable between WT and Tim-3 deficient DC (Extended FIG. 9c).

Although exogenous ligands can lead to inflammasome activation, including dsDNA from dying tumor cells and AIM2 inflammasome⁴⁰, it was hypothesized that potential endogenous or DC intrinsic DAMPs could also be driving inflammasome activation in tumor-infiltrating Tim-3^{cko} DC. Therefore, the inventors examined NLRP3, which has been shown to be activated by a number of endogenous ligands arising from a range of cellular stressors including reactive oxygen species (ROS)⁴¹⁻⁴⁴. Indeed, Tim-3^{cko} migDC scored more highly on a gene signature associated with oxidative stress (FIG. 4G; p=1.9*10⁻⁰⁶), including upregulation of key enzymes (SOD1, SOD2, BAG3, PRDX5)^{45,46} involved in antioxidant defenses to negatively regulate accumulation of reactive oxygen species (FIG. 4H). As ROS is a hallmark of oxidative stress and a known ligand of NLRP3, the inventors investigated the ROS activity of DCs from MC38-OVA tumors and found significantly elevated ROS activity in Tim-3 deficient DC1 and MigDC (FIG. 4I).

To investigate the effect of oxidative stress in Tim3^{cko} tumor bearing animals the inventors implanted Tim3^{fl.fl} and Tim3^{fl.fl}Zbtb46 cre mice with MC38-OVAdim and treated with or without antioxidant N-Acetyl-Cysteine (NAC) for the duration of the experiment. Strikingly the protective inhibition of tumor growth in Tim3^{cko} was completely abrogated in Tim3^{cko} mice receiving NAC, as assessed by tumor growth (FIG. 4J) and tumor weights post-harvest (FIG. 4K). To determine whether mitigation of oxidative stress in Tim3^{cko} tumors also affected IL-1β levels intratumorally, the inventors prepared tumor supernatants and determined the levels of IL-1β in WT and Tim3cko animals treated with or without NAC. In line with previous observations, Tim3^{cko} tumor had significantly elevated levels of IL-1β, and this was completely reversed in mice who had received NAC (FIG. 4L).

Finally, given that these results implicated a role for inflammasome activation in driving the anti-tumor immunity observed in Tim-3^{cko} mice, the inventors utilized three different approaches to target this pathway: (1) Caspase 1 inhibitor to interfere with pro-IL1β/IL-18 cleavage, (FIGS. 4M, 4N); (2) CRID3 to perturb ASC oligomerisation⁴⁷ (FIGS. 4O, 4P); and (3) anti- IL-1β/IL-18 to inhibit the effector cytokines downstream of inflammasome activation (FIG. 4Q, 4R). Each of the three approaches abolished the protective anti-tumor immunity observed in the Tim-3^{cko}, with anti-IL-1β/IL-18 treatment having the most significant effect in abrogating the protective effect as measured by both tumor size (FIG. 4Q) and weight (FIG. 4R) in both MC38-OVA and B16-OVA (Extended Data. FIG. 9D). Flow cytometric analysis of the treated Tim-3^{cko} tumors showed complete reversal of the CD8+ phenotype observed in Tim3^{cko} tumors, including loss of the expanded CD8+PD1+ population (FIG. 4S) and the observed increased TCF1 (FIG. 4T). These results build upon recent findings whereby administration of an engineered decoy receptor resistant IL-18 led to expansion of TCF1+CD8 T cells in the TME⁴⁸. Together with these findings, these data highlight the role that innate cytokines such as IL-1 and IL-18 can play beyond T cell priming, and asserts their importance in curtailing an exhaustion trajectory, sustaining effector T cell responses responsible for anti-tumor immunity. The inventors further found complete reversal of proliferative capacity-Ki67 (FIG. 4U), and production of IL-2 (FIG. 4V) and IFN-γ (FIG. 4W) upon blockade of IL-1/IL-18 in Tim3^{cko}.

All data were additionally confirmed in another tumor-B16-OVA (Extended Data FIG. 9E-J). Interestingly, Granzyme B (data not shown) remained unaffected by inflammasome inhibition indicating that there are additional pathways regulating the full cytotoxicity of CTL responses in Tim-3^{cko} tumors.

Taken together, these findings reveal a dominant role for Tim-3 in regulating DC function in promoting anti-tumor immunity. Previous pre-clinical studies using Tim3 mAbs have also observed high expression of Tim-3 on intratumoral DCs and concluded that the therapeutic efficacy of anti-Tim3 was likely to be mediated by DC1s10. However, many immune checkpoint therapies fail in Batf3 KO and Zbtb46-DTR, and considering that Tim-3 is expressed by multiple cell types, possessing many different roles, including DCs9, ILCs49, Th14, Th1750, CD8+2 and Tregs5, this makes any interpretation from mAb therapy studies challenging, and has confounded prior understanding about the precise role of Tim-3 in these diverse cell types.

To address this, the inventors implanted Tim3^{fl.fl}, Tim3^{fl.fl}CD4 cre and Tim3^{fl.fl}Zbtb46 cre with B16-OVA tumor cells and treated therapeutically with either i) Isotype controls, ii) anti-Tim-3, iii) anti PDL1 or iv) anti-Tim-3+PDL1. It was observed that WT (Tim3^{fl.fl}) recipients showed modest reduction in tumor burden with either anti-Tim-3 or anti-PDL1 as a monotherapy, with a significant reduction in tumor growth when used in combination (Extended FIG. 9k). While the additive effect of anti-Tim-3 with anti-PDL1 was also observed in Tim3^{fl.fl}CD4 cre tumor bearing mice, (Extended FIG. 9L), the therapeutic effect of combined blockade with anti-Tim-3+ anti-PD-L1 was completely lost in the mice where Tim-3 was deleted on DCs using the Zbtb46cre (Extended FIG. 9m). These data indicate that anti-Tim-3 antibody primarily works on DCs in the TME to induce anti-tumor immunity.

Taken together, the data in this study show that loss of Tim-3 prevents migratory DC from acquiring a regulatory program and facilitates the maintenance of the CD8+ effector T cell pool driven by IL-1 family member cytokines IL-1γ and IL-18. These results build on growing interest in enhancing inflammasome pathways in cancer⁵¹, and underscores the value of Tim-3 blockade in liberating inflammasome activity to potentiate anti-tumor immunity. It is also noteworthy that in AML, leukemic cells intrinsically have higher levels of oxidative stress and ROS production, both of which increase in response to chemotherapy⁵². Tim-3 has been identified as a specific marker for leukemic stem cells (LSCs) in AML¹³, with particularly high expression in patients with refractory AML¹⁴. Therefore, TIM-3 has become an attractive therapeutic target, and emerging data indicate that Tim-3 blockade in AML and MDS can induce 50-60% response rates¹⁵, paving the way for developing TIM-3 blockade for myeloid tumors where other checkpoint blockade therapies have had limited success⁵³⁻⁵⁵.

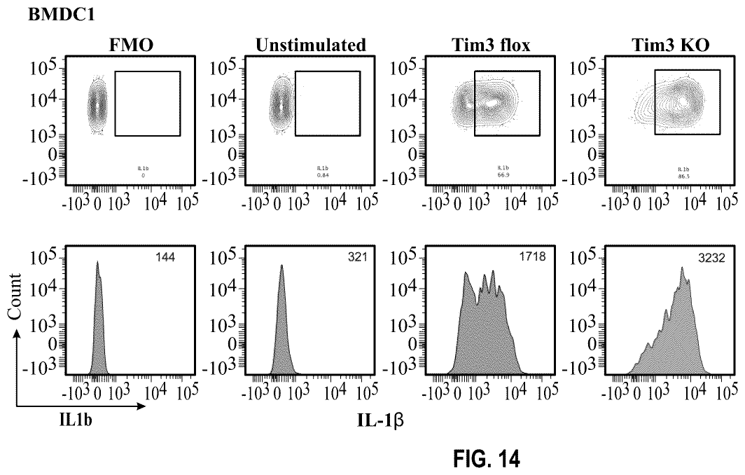

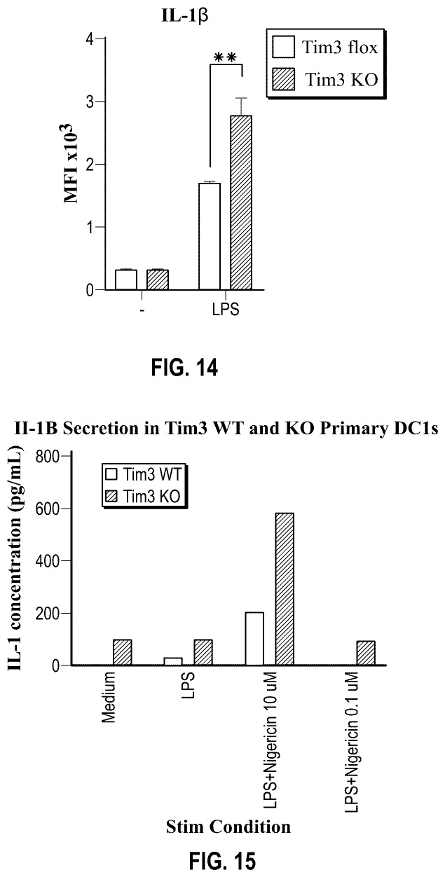

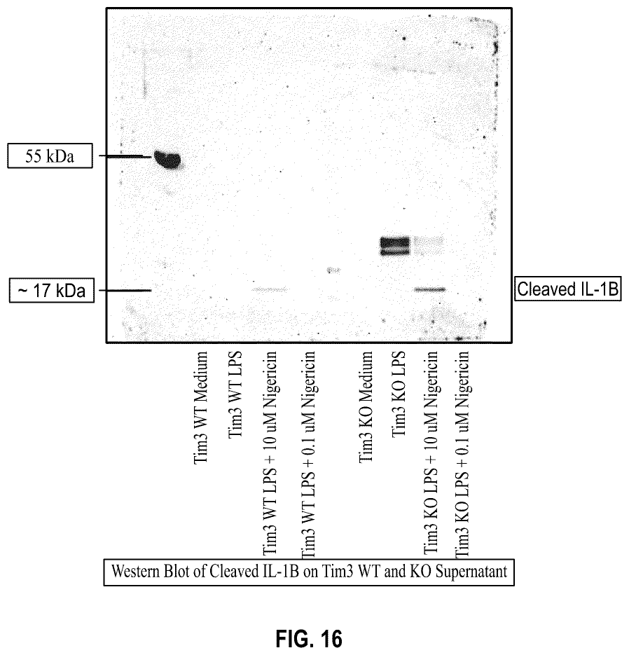

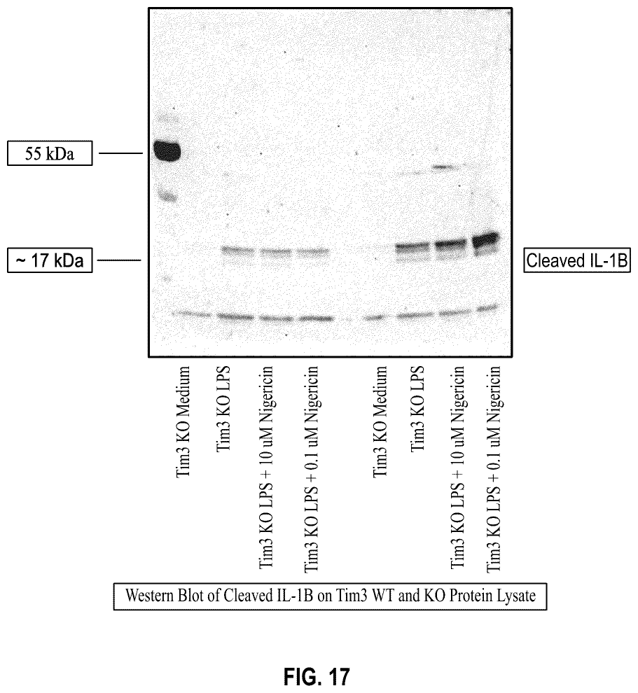

Enhanced inflammasome activation in Tim-3^{cko} DC. BMDC were differentiated in the presence of FLT3L for 10 days (FIG. 14). Flow cytometric analysis assessing IL-1β in WT or Tim3 KO DC1. XCR1+ cells were sorted after 10 days of differentiation and seeded at a density of 0.25×10⁶. Sorted cells were either unstimulated or primed with LPS (1 ug·ml) for 3 hours followed by flow cytometric analysis assessing IL-1β. BMDC were differentiated in the presence of FLT3L for 10 days, XCR1+ cells were sorted after 10 days of differentiation and seeded at a density of 1×10⁶. Sorted cells were either unstimulated or primed with LPS (1 ug/ml) for 3 hours followed by addition of Nigericin (10 mM). Following 30 mins stimulation, culture supernatants were harvested and ELISA was performed to detect IL-1β by ELISA (FIG. 15), or Western Blot (FIG. 16). For determination of cleaved IL-1β in cultures supernatant was incubated over night with 0.5 ug IL-1β Ab and 20 ul streptavidin beads. Beads were resuspended in 1× SDS buffer and supernatant was isolated, and full volume was directly run on the blot. Whole protein lysates from WT and Tim3 KO DC1 were also probed for cleaved IL-1β (FIG. 17). Tim-3 deficient DC are more primed for inflammasome activation with increased levels of cleaved IL-β production in cellular supernatants and protein lysates.

These data indicate that Tim3 curtails inflammasome activation and cell death through pyroptosis.

### Methods

Mice: 6-8-week-old C57BL/6, CD11c cre, Zbtb46 cre, CD4 cre, E8iCre, LysM-Cre, CX3CR1 cre and Foxp3ERT2 cre mice were purchased from the Jackson Laboratory. Tim-3^{fl/fl} mice were generated as described in supplementary materials. Tim3 conditional knockout mice were generated by crossing to the above cres. For KI/KO alleles (LysM cre) the appropriate controls were used i.e., Tim3^{fl/wt}xLysM cre^{+/-}, for all other cre lines flox/flox mice were used as controls. For experiments with Foxp3ERT2 cre mice, animals were orally gavaged with 8 mg Tamoxifen 3 days prior to tumor implantation and every 3 days thereafter for the duration of the experiments. Deletion efficiency was determined by flow cytometry (not shown). Animal experiments were done in accordance with the guidelines of the Institutional Animal Care and Use Committee (IACUC) at Brigham and Women's Hospital and Harvard Medical School.

### Gene-Targeted Deletion of Murine Tim-3 Locus

To generate Tim-3 floxed mice on the C57BL/6 background, targeting vectors containing genomic fragments of the havcr2 (encoding Tim-3) gene were constructed by using C57BL/6 BAC clones. Linearized targeting vector was transfected into B6 embryonic stem (ES) cells. Homologous recombinants were identified by Southern-blot analysis and implanted into foster B6-albino mothers. Chimeric mice were bred to C57BL/6 mice, and the F1 generation was screened for germline transmission. The Neo gene was removed by breeding F1 mice with a strain of actin promoter driven Flipase transgenic mice (Jackson Laboratory).

### Tumor Induction

The C57BL/6-derived colon carcinoma MC38 and MC38-OVAdim were maintained at 37° C. with 5% CO₂ in DMEM medium supplemented with 10% heat-inactivated fetal calf serum, penicillin, and streptomycin and 1% Sodium Pyruvate. The C57BL/6-derived melanoma cell lines-B16 and B16-OVA-transfected clone were maintained at 37° C. with 5% CO₂ in RPMI medium supplemented with 10% heat-inactivated fetal calf serum, penicillin, and streptomycin. MC38- OVA^{dim} (0.5×10⁶), B16F10/B16-OVA (0.25×10⁶) and MC38 (1.0×10⁶) cells were injected subcutaneously in 100 μl PBS on the flank. Tumor size was determined by the formula L×W where L=length, W=width on the indicated days. In some experiments, mice were treated with anti-IL-1β (B122) and anti-IL-18 (YIGIF74-1 G7) or appropriate control Hamster Ig and Rat IgG2a (all at 8 mg/kg) i.p. on days 3, 6, 9 post tumor implantation. For immune checkpoint experiments anti-Tim-3 was administered at a dose of 8 mg/kg and anti-PDL1 at a dose of 2 mg/kg, Isotype controls were used at the same concentration in control animals. Neutralizing anti IL-4 was used at 1.25 mg/kg, anti-IL-12 at 25 mg/kg. For CRID3 experiments mice were treated with CRID3 (R & D) at a dose of 8 mg/kg or PBS as control i.p. on days 0, 2, 4, 6, 8, 10 until experiment termination. For Caspase 1 inhibitor (Invivogen) mice were orally gavaged with inhibitor or control (water) at a dose 8 mg/kg on days 0, 2, 4, 6, 8, 10 until experiment termination. For NAC experiments mice were given NAC daily in drinking water at a concentration of 40 mM.

### Lung Tumor

Lung tumor experiments were conducted using orthotopic syngeneic KP1.9 lung adenocarcinoma cells. These cells have been derived from lung tumors of C57BL/6 KP mice harboring Kras and Trp53 mutations⁵⁶. Tumor cells were injected intravenously (i.v. 2.5×10⁵ cells in 100 μl PBS) to develop orthotopic tumors. Animals reproducibly show macroscopic lung tumor nodules at 3-4 weeks post injection. Evaluation of lung tumor burden was assessed by histological analyses based on hematoxylin and eosin (H&E) staining of explanted lung tissue harvested 4 weeks post implantation. For quantification, tumor area was calculated as a percentage of area occupied by the tumor as a part of total lung tissue.

### Preparation of Cell Suspensions

Single cell suspension was obtained after tumor digestion with 50 ug/ml Liberase™ (Roche)+20 ug/ml DNAse I (Roche) at 37° C. for 30 minutes. Haematopoietic cells were enriched by density gradient centrifugation with 30% Percoll (GE Healthcare Life Sciences) for 20 min at 1800 rpm (no brake) followed by passing through a 40-μm cell strainer. DCs were obtained from LNs after 50 ug/ml Liberase™ (Roche)+20 ug/ml DNAse I (Roche) at 37° C. for 30 minutes.

### Flow Cytometry

Single cell suspensions were incubated with anti-CD 16/32 (BioXcell) for 15 min at 4° C., followed by staining with fluorescent conjugated Abs for 30-45 min at 4° C. Following cell populations were identified based on cell marker expression: neutrophils (CD45⁺CD11b⁺Ly6C⁻Ly6G⁺), monocytes (CD45⁺CD11b⁺Ly6G⁻Ly6C^{high}), CD11b⁺ macrophage (CD45⁺CD11b⁺Ly6G⁻Ly6C⁻F4/80⁺), T cells (CD45⁺CD3⁺CD4⁺ or CD8⁺), B cells (CD45⁺B220⁺CD19⁺), NK cells (CD45⁺CD3⁻NK1.1⁺), DC1 (CD45⁺, CD3⁻, CD19⁻, NK1.1⁻, ClassII⁺CD11c⁺CD64⁻CD11b⁻CD103/XCR1⁺), DC2 (CD45⁺, CD3⁻, CD19⁻, NK1.1⁻, ClassII⁺CD11c⁺CD64⁻CD11b⁺CD103/XCR1⁻), Mig DC DC1 (CD45⁺, CD3⁻, CD19⁻, NK1.1⁻, ClassII⁺CD11c⁺CD64⁻CD11b⁺CD103/XCR1⁺). The following antibodies (Biolegend & BD) were used: CD45 (30-F11), CD4 (RM4-5), CD8a (53-6.7), B220 (RA3-6B2), NK1.1 (PK136), CD11b (M1/70), CD11c (N418), CD103 (P84), XCR1 (ZET), CD64 (X54-5/7.1), CD86 (GL-1), PDL1 (10F.9G2), CD27 (LG.3A10), IL-7Ra (A7R34), CXCR5 (L138D7), CX3CR1 (SA011F11), CD5 (53-7.3), CD24 (M1/69), PDL2 (122), Lag3 (C9B7W), TIGIT (GIGD7), Tim3 (5D12), Ly6C (HK1.4), Ly6G (1A8), TCRb (H57-597), F4/80 (BM8), Ki67 (16A8), T-bet (4B10), TCF1 (C63D9).

### Re-Stimulation and Intracellular Cytokine Staining

For intra-cellular cytokine (ICC) staining cells were stimulated with phorbol-12-myristate 13- acetate (PMA) (50 ng/ml, Sigma-Aldrich, MO) and ionomycin (1 ug/ml, Sigma-Aldrich, MO) in the presence of Golgi Plug (BD Biosciences) and Golgi Stop (BD Biosciences) for four hours prior to cell surface and ICC staining. Antibody detecting CD107a (1D4B) was added to the cells during cell culture incubation. Following fixation and permeabilization, staining with antibodies against the following was performed with the following antibodies purchased from Biolegend or BD Biosciences: IL-2 (JES6-5H4), TNF-a (MP6-XT22), IFN-γ (XMG-1.2), Granzyme B (GB11), IL-10 (JES5-16E3), Perforin (eBioOMAK-D), IL-12p40 (C17.8), IL-27p28 (MM27-7B1), LAP (TW7-20B9), CXCL9 (MIG-2F5.5). Antigen specific T cells were determined by H-2Kb/OVA257-264 dextramer staining following the manufacturer's protocol (Immudex). All data were collected on a BD Fortessa or Symphony (BD Biosciences) and analyzed with FlowJo 10.4.2 software (TreeStar).

### In Vitro Bone-Marrow-Derived Dendritic-Cell Cultures and DC Transfer

Bone-marrow cells were isolated by flushing femurs, tibias and humeri with PBS, supplemented with 0.5% BSA, 2 nM EDTA, and 1% penicillin/streptomycin (P/S). Bone-marrow cells were strained through a 70-μm filter and centrifuged before resuspension in 1× Ack lysis buffer (Gibco) for 5 min on ice. For generation of DC2 cells were plated in RPMI medium with 10% fetal calf serum (FCS), 1% L-glutamine, 1% sodium pyruvate, 1% MEM non-essential amino acids, 1% P/S, 55 μM 2-mercaptoethanol 10 ng/ml GMCSF (Peprotech) and 5 ng/ml IL-4 (R&D). For DC1 cells were plated as above but with 200 ng/ml recombinant Flt-3 ligand (Biolegend). DCs were analyzed on days 7-9, for transfer experiments cells were sorted for expression of XCR1/CD103 (DC1) or Sirpa (DC2). On day 3 after tumor implantation DCs were transferred (0.5×10⁶) per mouse in 50 ul PBS subcutaneously adjacent to the tumor. For sham control mice PBS alone was injected.

### In-Vitro Cross Presentation Assays

Splenic DC1s were cultured with Cell Trace Violet (CTV) labelled OT-I cells together with i) HLA mismatched apoptotic splenocytes osmotically loaded with ovalbumin, or ii) latex beads passively coupled with ovalbumin. Non-ova loaded dead cells or non-ova coupled beads served as control respectively. After 72 hours, co-culture the proliferation of CD8+ T cells was assessed by flow cytometry where expression of CD44 together with dilution of CTV dye was indicative of activation and proliferation.

### In Vivo Killing Assay

On D12 of tumor growth mice were injected intravenously (i.v.) with splenocytes differentially labeled with CFSE or CTV (Invitrogen) and loaded with 100 nM OVA257-264 or MOG37-46 (Irrelevant Antigen). Cells were injected at 50:50 ratio into MC38-OVA bearing WT or Tim3^{cko} mice respectively and collected 24 hr after injection. Percentage cytotoxicity calculated as 100—(CTV/CTV+CFSE).

### ROS Activity Assay

Single cell suspensions were generated as described and cells were resuspended in HBSS containing 0.1% BSA followed by FACS antibody surface marker staining for 30 min on ice. Cells were then washed and resuspended in PBS-EGG buffer (1 mM EDTA, 0.05% gelatin, 0.09% glucose) and 5.0 μM DHR123 probe (Thermo Fisher Scientific) was added for 30 min at 37° C. The reaction was stopped by placing the cells on ice and washing with PBS-EGG buffer. Cells were resuspended in PBS containing 0.1% BSA and activated rhodamine 123 signal (activated DHR 123) was analyzed in the FITC channel on a BD Symphony within 30 min. Hydrogen peroxide was added to some samples to serve as a positive control.

### Inflammasome Activation

BMDC1 were generated as described above. XCR1+ cells were sorted after 10 days of differentiation and seeded at a density of 0.5×10⁶. Sorted cells were either unstimulated or primed with LPS for 3 hours followed by the addition of ATP (5 mM; Sigma), Oxidized phospholipids (ox-PAPC) (100 ug·ml; Invivogen) or pdA:dT (1 ug/ml; Invivogen). Following overnight cultures supernatants were harvested and ELISA was performed to detect IL-1β and TNF-α (non-inflammasome regulated control).

### Statistical Analysis

Graphs were compiled and statistical analyses were performed with Prism software (GraphPad). Statistical significance was evaluated with the Two-Tailed Unpaired t-test when comparing two groups and Two-way ANOVA when comparing more than two groups. In all cases, p values *p<0.05, **p<0.01, ***p<0.001, ****p<0.0001, n.s. not significant.

### Multiplexing Samples by Cell-Hashing

After flow sorting, cells from each sample (Tumor or dLN or ndLN from an individual mouse) was resuspended in PBS containing 2% FCS and stained with oligo tagged TotalSeq antibodies directed against CD45 for 30 minutes on ice. Cells were washed and pooled accordingly, centrifuged at 1200 rcf for 5 minutes at 4 degrees and resuspended in PBS+2% FCS. For each 10× channel 6 samples were combined: WT1 -Tumor, dLN, ndLN; KO1-Tumor, dLN, ndLN; WT2-Tumour, dLN, ndLN; KO2-Tumour, dLN, ndLN.

### Single-Cell RNA-Seq Library Preparation

Droplet-based 3′ end massively parallel single-cell RNA sequencing (scRNAseq) was performed by encapsulating sorted live CD45+ tumor infiltrating cells into droplets and libraries were prepared using Chromium Single Cell 5′ Reagent Kits v2 according to manufacturer's protocol (10× Genomics). The generated scRNAseq libraries were sequenced using an Illumina HiSeq2500 to an average depth of 60.4 million paired end reads per sample.

### Single-Cell RNA Sequencing Data Analysis

The following preprocessing steps were conducted: generating, demultiplexing FASTQ files from the raw sequencing reads (belfastq, v2.20), aligning to UCSC mm10 mouse transcriptome (cellranger, v3.0.2, 10× Genomics), demultiplexing cell-hashing data, and generating of raw gene count matrices using Cumulus (v0.8.0), a cloud based data analysis framework57 (Nature Methods; accepted). The pipelines were run in Terra Cloud Platform⁵⁷ (available on the world wide web at app.terra.bio). Downstream analysis was performed using the R software package Seurat⁵⁸ (v2.3.4, available on the world wide web at satijalab.org/seurat/). Low-quality cells with less than 500 genes detected were removed from the analysis as were and genes expressed in fewer than 50 cells. The filtered raw gene expression matrix was cell- normalized over total number of counts, multiplied by 10,000 and log transformed. Principal Component Analysis (PCA) was performed on a submatrix of the top 1,000 most variable genes (computed using FindVariableGenes from Seurat), and the number of top principal components for further analysis was determined by the elbow method, keeping 12 and 15 PCs for analysis of tumor only or all cells (plus dLN, ndLN). Cells were clustered using a shared nearest neighbor (SNN) modularity optimization-based clustering algorithm as implemented in a function FindClusters from the Seurat package. Cluster-specific differentially expressed genes were computed using function FindAllMarkers from Seurat with default test parameters. Gene expression values, gene signature scores, and clustering results were visualized by embedding cells in a Uniform Manifold Approximation and Projection59 (UMAP) of the same dimensionally-reduced space as in clustering of cells. Annotating cell clusters was performed by assessing known cell- type markers and comparison with public myeloid tumor datasets^{17,20,21}.

### Creating Gene Signatures

Curated gene signatures were constructed from various databases of gene signatures (data not shown). Il-1 signature was determined from differential genes between Il1b treated and vehicle CD8+ T cells⁶⁰. The expression matrix was created from data downloaded from GEO database, accession number GSE127234. The inventors used edgeR61 to compute the differentially expressed genes, and the Il-1 signature was created from top genes upregulated in Il1b treated cells (logFC>3, FDR<0.1). Given a set of genes, signature score activity levels were computed using the AddModuleScore function in Seurat with the parameters defining the control gene-sets specified by n.bin=25 and ctrl.size=100. The difference in gene signature score activity levels between WT and cKO was examined using Wilcoxon Rank Sum test and visualized using violin or empirical cumulative distribution function (ECDF) plots.

### Determining Potential Receptor-Ligand Interactions

To characterize potential paracrine signaling between MigDC and CD8+ T cells, a method proposed by Schiebinger et al³⁷ was used. In particular, the inventors defined a list of ligands from the following GO terms: cytokine activity (GO:0005125), growth factor activity (GO:0008083), and hormone activity (GO:0005179). The set of receptors was defined by the GO term receptor activity (GO:0004872). The inventors determined the set of potential ligand-receptors pairs using a curated database of mouse protein-protein interactions⁶². Potential ligand-receptor interactions were quantified by an interaction score defined as the product of the fraction of cells in the cell cluster expressing ligand and the fraction of cells in the cell cluster expressing the cognate receptor. To remove ubiquitously expressed ligands and receptors, the inventors used 10,000 permutations to generate empirical null distribution of interaction scores by randomly shuffling the cell labels.

Each ligand-receptor interaction score was standardized by computing the distance between the interaction score and the mean interaction score in units of standard deviations from the permuted data as described³⁷.

### Data Availability

Data has been uploaded to NCBI Gene Expression Omnibus and is available on the world wide web at ncbi.nlm.nih.gov/geo/ under data repository accession number GSE151914.

### REFERENCES

1. Monney, L. et al. Th1-specific cell surface protein Tim-3 regulates macrophage activation and severity of an autoimmune disease. Nature 415, 536-541, doi: 10.1038/415536a [pii] (2002).
2. Sakuishi, K. et al. Targeting Tim-3 and PD-1 pathways to reverse T cell exhaustion and restore anti-tumor immunity. J Exp Med 207, 2187-2194, doi:10.1084/jem.20100643 (2010).
3. Jin, H. T. et al. Cooperation of Tim-3 and PD-1 in CD8 T-cell exhaustion during chronic viral infection. Proc Natl Acad Sci U S A 107, 14733-14738, doi: 1009731107 [pii] 10.1073/pnas. 1009731107 (2010).
4. Rangachari, M. et al. Bat3 promotes T cell responses and autoimmunity by repressing Tim-3-mediated cell death and exhaustion. Nat Med 18, 1394-1400, doi:nm.2871 [pii] 10.1038/nm.2871 (2012).
5. Sakuishi, K. et al. TIM3(+)FOXP3(+) regulatory T cells are tissue-specific promoters of T-cell dysfunction in cancer. Oncoimmunology 2, e23849, doi:10.4161/onci.23849 (2013).
6. da Silva, I. P. et al. Reversal of NK-cell exhaustion in advanced melanoma by Tim-3 blockade. Cancer Immunol Res 2, 410-422, doi: 10.1158/2326-6066.CIR-13-0171 (2014).
7. Anderson, A. C. et al. Promotion of tissue inflammation by the immune receptor Tim-3 expressed on innate immune cells. Science 318, 1141-1143, doi:10.1126/science.1148536 (2007).
8. Liu, L. Z. et al. CCL15 Recruits Suppressive Monocytes to Facilitate Immune Escape and Disease Progression in Hepatocellular Carcinoma. Hepatology 69, 143-159, doi:10.1002/hep.30134 (2019).
9. Broz, M. L. et al. Dissecting the tumor myeloid compartment reveals rare activating antigen-presenting cells critical for T cell immunity. Cancer Cell 26, 638-652, doi:10.1016/j.ccell.2014.09.007 (2014).
10. de Mingo Pulido, A. et al. TIM-3 Regulates CD103(+) Dendritic Cell Function and Response to Chemotherapy in Breast Cancer. Cancer Cell 33, 60-74 e66, doi: 10.1016/j.ccell.2017.11.019 (2018).
11. Chiba, S. et al. Tumor-infiltrating DCs suppress nucleic acid-mediated innate immune responses through interactions between the receptor TIM-3 and the alarmin HMGB1. Nat Immunol 13, 832-842, doi:10.1038/ni.2376 (2012).
12. Gayden, T. et al. Germline HAVCR2 mutations altering TIM-3 characterize subcutaneous panniculitis-like T cell lymphomas with hemophagocytic lymphohistiocytic syndrome. Nat Genet 50, 1650-1657, doi: 10.1038/s41588-018-0251-4 (2018).
13. Kikushige, Y. et al. TIM-3 is a promising target to selectively kill acute myeloid leukemia stem cells. Cell Stem Cell 7, 708-717, doi:10.1016/j.stem.2010.11.014 (2010).
14. Dama, P., Tang, M., Fulton, N., Kline, J. & Liu, H. Gal9/Tim-3 expression level is higher in AML patients who fail chemotherapy. J Immunother Cancer 7, 175, doi: 10.1186/s40425- 019-0611-3 (2019).
15. Uma Borate, M., Jordi Esteve, Kimmo Porkka, Steve Knapper, Norbert Vey, MD PhD, Sebastian Scholl, Guillermo Garcia-Manero, MD, Martin Wermke, MD, Jeroen Janssen, Elie Traer, Chong Chyn Chua, Rupa Narayan, Natalia Tovar, Mika Kontro, Oliver Ottmann, Prof, MD, Haiying Sun, Tyler Longmire, Sebastian Szpakowski, Serena Liao, Anuradha Patel, Mikael L Rinne, Andrew Brunner, MD, Andrew H. Wei, PhD FRACP, FRCPA, MBBS. Phase Ib Study of the Anti-TIM-3 Antibody MBG453 in Combination with Decitabine in Patients with High-Risk Myelodysplastic Syndrome (MDS) and Acute Myeloid Leukemia (AML). Blood 134, 570 (2019).
16. Roberts, E. W. et al. Critical Role for CD103(+)/CD141(+) Dendritic Cells Bearing CCR7 for Tumor Antigen Trafficking and Priming of T Cell Immunity in Melanoma. Cancer Cell 30, 324-336, doi:10.1016/j.ccell.2016.06.003 (2016).
17. Maier, B. et al. A conserved dendritic-cell regulatory program limits antitumour immunity. Nature 580, 257-262, doi: 10.1038/s41586-020-2134-y (2020).
18. Polprasert, C. et al. Frequent germline mutations of HAVCR2 in sporadic subcutaneous panniculitis-like T-cell lymphoma. Blood Adv 3, 588-595, doi: 10.1182/bloodadvances.2018028340 (2019).
19. Pfirschke, C. et al. Immunogenic Chemotherapy Sensitizes Tumors to Checkpoint Blockade Therapy. Immunity 44, 343-354, doi: 10.1016/j.immuni.2015.11.024 (2016).
20. Gubin, M. M. et al. High-Dimensional Analysis Delineates Myeloid and Lymphoid Compartment Remodeling during Successful Immune-Checkpoint Cancer Therapy. Cell 175, 1014-1030 e1019, doi: 10.1016/j.cell.2018.09.030 (2018).
21. Zilionis, R. et al. Single-Cell Transcriptomics of Human and Mouse Lung Cancers Reveals Conserved Myeloid Populations across Individuals and Species. Immunity 50, 1317-1334 e1310, doi: 10.1016/j.immuni.2019.03.009 (2019).
22. Sade-Feldman, M. et al. Defining T Cell States Associated with Response to Checkpoint Immunotherapy in Melanoma. Cell 175, 998-1013 e1020, doi:10.1016/j.cell.2018.10.038 (2018).
23. Best, J. A. et al. Transcriptional insights into the CD8(+) T cell response to infection and memory T cell formation. Nat Immunol 14, 404-412, doi: 10.1038/ni.2536 (2013).
24. Kurtulus, S. et al. Checkpoint Blockade Immunotherapy Induces Dynamic Changes in PD-1(−)CD8(+) Tumor-Infiltrating T Cells. Immunity 50, 181-194 e186, doi:10.1016/j.immuni.2018.11.014 (2019).
25. Yost, K. E. et al. Clonal replacement of tumor-specific T cells following PD-1 blockade. Nat Med 25, 1251-1259, doi: 10.1038/s41591-019-0522-3 (2019).
26. Thommen, D. S. et al. A transcriptionally and functionally distinct PD-1(+) CD8(+) T cell pool with predictive potential in non-small-cell lung cancer treated with PD-1 blockade. Nat Med 24, 994-1004, doi: 10.1038/s41591-018-0057-z (2018).
27. Siddiqui, I. et al. Intratumoral Tcf1(+)PD-1(+)CD8(+) T Cells with Stem-like Properties Promote Tumor Control in Response to Vaccination and Checkpoint Blockade Immunotherapy. Immunity 50, 195-211 e110, doi: 10.1016/j.immuni.2018.12.021 (2019).
28. Miller, B. C. et al. Subsets of exhausted CD8(+) T cells differentially mediate tumor control and respond to checkpoint blockade. Nat Immunol 20, 326-336, doi: 10.1038/s41590-019- 0312-6 (2019).
29. Philip, M. et al. Chromatin states define tumour-specific T cell dysfunction and reprogramming. Nature 545, 452-456, doi: 10.1038/nature22367 (2017)
30. Im, S. J. et al. Defining CD8+ T cells that provide the proliferative burst after PD-1 therapy. Nature 537, 417-421, doi: 10.1038/nature19330 (2016).
31. Pauken, K. E. et al. Epigenetic stability of exhausted T cells limits durability of reinvigoration by PD-1 blockade. Science 354, 1160-1165, doi: 10.1126/science.aaf2807 (2016).
32. Jadhav, R. R. et al. Epigenetic signature of PD-1+TCF1+CD8 T cells that act as resource cells during chronic viral infection and respond to PD-1 blockade. Proc Natl Acad Sci U S A116, 14113-14118, doi: 10.1073/pnas. 1903520116 (2019).
33. Gao, Y. et al. Control of T helper 2 responses by transcription factor IRF4-dependent dendritic cells. Immunity 39, 722-732, doi:10.1016/j.immuni.2013.08.028 (2013).
34. Dixit, A. et al. Perturb-Seq: Dissecting Molecular Circuits with Scalable Single-Cell RNA Profiling of Pooled Genetic Screens. Cell 167, 1853-1866 e1817, doi:10.1016/j.cell.2016.11.038 (2016).
35. Bottcher, J. P. et al. NK Cells Stimulate Recruitment of cDCI into the Tumor Microenvironment Promoting Cancer Immune Control. Cell 172, 1022-1037 e1014, doi:10.1016/j.cell.2018.01.004 (2018).
36. Spranger, S., Dai, D., Horton, B. & Gajewski, T. F. Tumor-Residing Batf3 Dendritic Cells Are Required for Effector T Cell Trafficking and Adoptive T Cell Therapy. Cancer Cell 31, 711- 723 e714, doi: 10.1016/j.ccell.2017.04.003 (2017).
37. Schiebinger, G. et al. Optimal-Transport Analysis of Single-Cell Gene Expression Identifies Developmental Trajectories in Reprogramming. Cell 176, 928-943 e922, doi:10.1016/j.cell.2019.01.006 (2019).
38. Martinon, F., Burns, K. & Tschopp, J. The inflammasome: a molecular platform triggering activation of inflammatory caspases and processing of proIL-beta. Mol Cell 10, 417-426, doi: 10.1016/s1097-2765(02)00599-3 (2002).
39. Zanoni, I. et al. An endogenous caspase-11 ligand elicits interleukin-1 release from living dendritic cells. Science 352, 1232-1236, doi:10.1126/science.aaf3036 (2016).
40. Hornung, V. et al. AIM2 recognizes cytosolic dsDNA and forms a caspase-1-activating inflammasome with ASC. Nature 458, 514-518, doi: 10.1038/nature07725 (2009).
41. Masters, S. L. et al. Activation of the NLRP3 inflammasome by islet amyloid polypeptide provides a mechanism for enhanced IL-1beta in type 2 diabetes. Nat Immunol 11, 897- 904, doi: 10.1038/ni.1935 (2010).
42. Martinon, F. Signaling by ROS drives inflammasome activation. Eur J Immunol 40, 616- 619, doi:10.1002/eji.200940168 (2010).
43. Schroder, K., Zhou, R. & Tschopp, J. The NLRP3 inflammasome: a sensor for metabolic danger? Science 327, 296-300, doi: 10.1126/science.1184003 (2010).
44. Gross, C. J. et al. K(+) Efflux-Independent NLRP3 Inflammasome Activation by Small Molecules Targeting Mitochondria. Immunity 45, 761-773, doi:10.1016/j.immuni.2016.08.010 (2016).
45. Chakraborty, D. et al. Enhanced autophagic-lysosomal activity and increased BAG3-mediated selective macroautophagy as adaptive response of neuronal cells to chronic oxidative stress. Redox Biol 24, 101181, doi: 10.1016/j.redox.2019.101181 (2019).
46. Rosati, A., Graziano, V., De Laurenzi, V., Pascale, M. & Turco, M. C. BAG3: a multifaceted protein that regulates major cell pathways. Cell Death Dis 2, e141, doi:10.1038/cddis.2011.24 (2011).
47. Coll, R. C. et al. A small-molecule inhibitor of the NLRP3 inflammasome for the treatment of inflammatory diseases. Nat Med 21, 248-255, doi: 10.1038/nm.3806 (2015).
48. Zhou, T. et al. IL-18BP is a secreted immune checkpoint and barrier to IL-18 immunotherapy. Nature, doi: 10.1038/s41586-020-2422-6 (2020).
49. Wallrapp, A. et al. Calcitonin Gene-Related Peptide Negatively Regulates Alarmin-Driven Type 2 Innate Lymphoid Cell Responses. Immunity 51, 709-723 e706, doi:10.1016/j.immuni.2019.09.005 (2019).
50. Hastings, W. D. et al. TIM-3 is expressed on activated human CD4+T cells and regulates Th1 and Th17 cytokines. Eur J Immunol 39, 2492-2501, doi: 10.1002/eji.200939274 (2009).
51. Zhivaki, D. et al. Inflammasomes within Hyperactive Murine Dendritic Cells Stimulate Long-Lived T Cell-Mediated Anti-tumor Immunity. Cell Rep 33, 108381, doi:10.1016/j.celrep.2020.108381 (2020).
52. Heasman, S. A., Zaitseva, L., Bowles, K. M., Rushworth, S. A. & Macewan, D. J. Protection of acute myeloid leukaemia cells from apoptosis induced by front-line chemotherapeutics is mediated by haem oxygenase-1. Oncotarget 2, 658-668, doi: 10.18632/oncotarget.321 (2011).
53. Zeidan, A. M. et al. A Multi-center Phase I Trial of Ipilimumab in Patients with

Myelodysplastic Syndromes following Hypomethylating Agent Failure. Clin Cancer Res 24, 3519-3527, doi:10.1158/1078-0432.CCR-17-3763 (2018).
54. Davids, M. S. et al. Ipilimumab for Patients with Relapse after Allogeneic Transplantation. N Engl J Med 375, 143-153, doi: 10.1056/NEJMoa1601202 (2016). Daver N et al. Phase IB/II Study of Nivolumab in Combination with Azacytidine (AZA) in Patients (pts) with Relapsed Acute Myeloid Leukemia (AML). Blood 128 (22), p. 763 (2016).
56. Engblom, C. et al. Osteoblasts remotely supply lung tumors with cancer-promoting SiglecF(high) neutrophils. Science 358, doi: 10.1126/science.aal5081 (2017).
57. Li, B., Joshua Gould, Yiming Yang, Siranush Sarkizova, Marcin Tabaka, Orr Ashenberg, Yanay Rosen et al. “Cumulus: a cloud-based data analysis framework for large-scale single-cell and single-nucleus RNA-seq.” bioRxiv (2019): 823682.
58. Butler, A., Hoffman, P., Smibert, P., Papalexi, E. & Satija, R. Integrating single-cell transcriptomic data across different conditions, technologies, and species. Nat Biotechnol 36, 411-420, doi:10.1038/nbt.4096 (2018).
59. Becht, E. et al. Dimensionality reduction for visualizing single-cell data using UMAP. Nat Biotechnol, doi: 10.1038/nbt.4314 (2018).
60. Lee, P. H. et al. Host conditioning with IL-1beta improves the antitumor function of adoptively transferred T cells. J Exp Med 216, 2619-2634, doi:10.1084/jem.20181218 (2019).
61. Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).

62. Mertins, P. et al. An Integrative Framework Reveals Signaling-to-Transcription Events in Toll-like Receptor Signaling. Cell Rep 19, 2853-2866, doi:10.1016/j.celrep.2017.06.016 (2017).

1. A composition for selectively promoting inflammasome activity in myeloid cells, the composition comprising a TIM-3 inhibitor linked to an agent that specifically binds a myeloid cell surface marker.

2. The composition of claim 1, wherein the myeloid cell is a myeloid progenitor cell, a basophil, a neutrophil, an eosinophil, a monocyte, a macrophage, a dendritic cell, a granulocyte, a megakaryocyte or any combination thereof.

3. The composition of claim or claim 2, wherein the TIM-3 inhibitor specifically binds to TIM-3.

4. The composition of any one of claims 1-3, wherein the TIM-3 inhibitor in the composition more efficiently promotes myeloid cell inflammasome activity than the TIM-3 inhibitor not linked to the agent that binds a myeloid cell surface marker.

5. The composition of any one of claims 1-4, wherein the TIM-3 inhibitor in the composition more efficiently promotes tumor cell death than the TIM-3 inhibitor not linked to the agent that binds a myeloid cell surface marker.

6. The composition of claim 5, wherein the tumor cell death comprises pyroptosis.

7. The composition of any one of claims 1-6, wherein the TIM-3 inhibitor comprises an antibody or antigen-binding fragment thereof that specifically binds TIM-3.

8. The composition of claim 7, wherein the antibody or antigen-binding fragment thereof binds an epitope on the extracellular domain of TIM-3.

9. The composition of any one of claims 1-8, wherein the TIM-3 inhibitor promotes degradation of TIM-3 or RNA encoding TIM-3.

10. The composition of any one of claims 1-6, wherein the TIM-3 inhibitor comprises an RNA interference (RNAi) molecule, an antisense molecule, or a small molecule.

11. The composition of any one of claims 1-10, wherein the TIM-3 inhibitor is in or on a nanoparticle.

12. The composition of any one of claims 1-11, wherein the myeloid cell surface marker is selected from CD47, CD11b and CD11c.

13. A pharmaceutical composition comprising the composition of any one of claims 1-12 and a pharmaceutically-acceptable carrier.

14. A nanoparticle comprising a TIM-3 inhibitor in or on the nanoparticle.

15. The nanoparticle of claim 14, wherein the TIM-3 inhibitor comprises a nucleic acid, a peptide or a small molecule.

16. The nanoparticle of claim 14 or 15, wherein the TIM-3 inhibitor comprises an antibody or antigen-binding fragment thereof that specifically binds TIM-3.

17. The nanoparticle of claim 14 or 15, wherein the TIM-3 inhibitor comprises a nucleic acid that promotes degradation of RNA encoding TIM-3.

18. The nanoparticle of claim 17, wherein the nucleic acid is selected from an RNAi molecule, an miRNA, a CRISPR/Cas gRNA, and an antisense molecule.

19. The nanoparticle of any one of claims 14-18, which comprises a lipid nanoparticle.

20. The nanoparticle of any one of claims 14-19, further comprising an agent that specifically binds to a myeloid cell surface marker.

21. A method of promoting inflammasome activity, the method comprising contacting myeloid cell with a composition of any one of claims 1-20.

22. The method of claim 21, wherein the inflammasome activity is induced to a greater extent than induced by a TIM-3 inhibitor lacking to an agent that specifically binds a myeloid cell surface marker.

23. The method of claim 21 or 22, wherein the myeloid cell is a myeloid progenitor cell, a basophil, a neutrophil, an eosinophil, a monocyte, a macrophage, a dendritic cell, a granulocyte, a megakaryocyte or any combination thereof.

24. The method of any one of claims 21-23, wherein the myeloid cell is in a solid tumor microenvironment.

25. A method of promoting cancer cell death, the method comprising contacting a myeloid cell associated with the cancer cell with a composition of any one of claims 1-20.

26. The method of claim 25, wherein inflammasome activity is induced to a greater extent than induced by a TIM-3 inhibitor lacking to an agent that specifically binds a myeloid cell surface marker.

27. The method of any one of claims claim 21-26, wherein the myeloid cell is a myeloid progenitor cell, a basophil, a neutrophil, an eosinophil, a monocyte, a macrophage, a dendritic cell, a granulocyte, a megakaryocyte or any combination thereof.

28. The method of any one of claims 25-27, wherein the cancer is acute myeloid leukemia (AML) or a solid tumor.

29. The method of any one of claims 25-28, wherein the cancer cells do not express TIM-3.

30. A method of treating cancer, the method comprising administering a composition of any one of claims 1-20 to a subject in need thereof.

31. The method of claim 30, wherein inflammasome activity in cancer-associated myeloid cells is induced to a greater extent than induced by a non-targeted TIM-3 inhibitor.

32. The method of claim 31, wherein the myeloid cell is a myeloid progenitor cell, a basophil, a neutrophil, an eosinophil, a monocyte, a macrophage, a dendritic cell, a granulocyte, a megakaryocyte or any combination thereof.

33. The method of any one of claims 30-32, wherein the cancer is acute myeloid leukemia (AML), chronic myeloid leukemia (CML) or a solid tumor.

34. The method of any one of claims 30-33, wherein cells of the cancer do not express TIM-3.

35. The method of any one of claims 30-34, wherein death of cells of the cancer is induced to a greater extent than induced by a TIM-3 inhibitor that is not linked to an agent that specifically binds a myeloid cell surface marker.

36. The method of any one of claims 30-35, wherein the cancer is a solid tumor.

37. The method of claim 36, wherein the microenvironment of the solid tumor is rendered less hostile to T cells by the administering.

38. The method of any one of claims 30-37, wherein the cancer is metastatic.

39. The method of any one of claims 30-38, wherein the cancer is angiogenic.

40. A composition comprising comprising a TIM-3 inhibitor linked to an agent that specifically binds a myeloid cell surface marker for use in promoting inflammasome activity or treating cancer in a subject.

41. The composition for use of claim 40, wherein the TIM-3 inhibitor specifically binds to TIM-3.

42. The composition for use of any one of claims 40-41, wherein the TIM-3 inhibitor in the composition more efficiently promotes myeloid cell inflammasome activity than the TIM-3 inhibitor not linked to the agent that binds a myeloid cell surface marker.

43. The composition for use of any one of claims 40-42, wherein the TIM-3 inhibitor in the composition more efficiently promotes tumor cell death than the TIM-3 inhibitor not linked to the agent that binds a myeloid cell surface marker.

44. The composition for use of any one of claims 40-43, wherein the TIM-3 inhibitor comprises an antibody or antigen-binding fragment thereof that specifically binds TIM-3.

45. The composition for use of claim 44, wherein the antibody or antigen-binding fragment thereof binds an epitope on the extracellular domain of TIM-3.

46. The composition for use of any one of claims 40-43, wherein the TIM-3 inhibitor promotes degradation of TIM-3 or RNA encoding TIM-3.

47. The composition for use of any one of claims 40-46, wherein the TIM-3 inhibitor comprises an RNA interference (RNAi) molecule, an antisense molecule, or a small molecule.

48. The composition for use of any one of claims 40-47, wherein the TIM-3 inhibitor is in or on a nanoparticle.

49. The composition for use of any one of claims 40-48, wherein the myeloid cell surface marker is selected from CD47, CD11b and CD11c. 50 The composition for use of any one of claims 40-49, further comprising a pharmaceutically acceptable carrier.

51. A composition comprising a TIM-3 inhibitor in or on the nanoparticle for use in promoting inflammasome activity or treating cancer in a subject.
